SEQUENCE LISTING (184 sequences)

SEQ ID NO: 1 - DNA, 38 bp, synthetic construct
  source 1..38: mol_type = other DNA
  modified_base 25..26: mod_base = OTHER; note = DNA
  modified_base order(1..24,27..38): mod_base = OTHER; note = RNA
  modified_base order(1..4,15..23,27,30..38): mod_base = OTHER; note = 2&apos;-O-methyl
  modified_base order(5..14,24,29): mod_base = OTHER; note = 2&apos;-O-methoxyethyl
  modified_base 28: mod_base = OTHER; note = 2&apos;-fluoro
  modified_base order(1..5,15..24,25^26,28..38): mod_base = OTHER; note = phosphorothioate
  modified_base 26^27: mod_base = OTHER; note = methylphosphonate
  modified_base order(5,8,13..14): mod_base = OTHER; note = m5c
  modified_base order(9..10,12): mod_base = OTHER; note = m5u
  modified_base 25: mod_base = OTHER; note = 6-amino-5-nitro-3-yl-2(1H)-pyridone nucleobase
  modified_base 26: mod_base = OTHER; note = Inosine
  modified_base 1..38: mod_base = OTHER; note = B1
ccaccgacttgtccttggtccaggnnagatgcaccttc

SEQ ID NO: 2 - DNA, 34 bp, synthetic construct
  source 1..34: mol_type = other DNA
  modified_base 21..22: mod_base = OTHER; note = DNA
  modified_base order(1..20,23..34): mod_base = OTHER; note = RNA
  modified_base order(1..10,20,25): mod_base = OTHER; note = 2&apos;-O-methoxyethyl
  modified_base order(11..19,23,26..34): mod_base = OTHER; note = 2&apos;-O-methyl
  modified_base 24: mod_base = OTHER; note = 2&apos;-fluoro
  modified_base order(11..20,21^22,24..34): mod_base = OTHER; note = phosphorothioate
  modified_base 22^23: mod_base = OTHER; note = methylphosphonate
  modified_base order(1,4,9..10): mod_base = OTHER; note = m5c
  modified_base order(5..6,8): mod_base = OTHER; note = m5u
  modified_base 21: mod_base = OTHER; note = 6-amino-5-nitro-3-yl-2(1H)-pyridone nucleobase
  modified_base 22: mod_base = OTHER; note = Inosine
  modified_base 1..34: mod_base = OTHER; note = B2
cgacttgtccttggtccaggnnagatgcaccttc

SEQ ID NO: 3 - DNA, 32 bp, synthetic construct
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..17,21,24..32): mod_base = OTHER; note = 2&apos;-O-methyl
  modified_base 22: mod_base = OTHER; note = 2&apos;-fluoro
  modified_base order(9..18,19^20,22..32): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..32: mod_base = OTHER; note = B3
acttgtccttggtccaggnnagatgcaccttc

SEQ ID NO: 4 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..31): mod_base = OTHER; note = 2&apos;-O-methyl
  modified_base 21: mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B4
cttgtccttggtccaggnnagatgcaccttc

SEQ ID NO: 5 - DNA, 30 bp, synthetic construct
  source 1..30: mol_type = other DNA
  modified_base 17..18: mod_base = OTHER; note = DNA
  modified_base order(1..16,19..30): mod_base = OTHER; note = RNA
  modified_base order(1..6,16,21): mod_base = OTHER; note = 2&apos;-O-methoxyethyl
  modified_base order(7..15,19,22..30): mod_base = OTHER; note = 2&apos;-O-methyl
  modified_base 20: mod_base = OTHER; note = 2&apos;-fluoro
  modified_base order(7..16,17^18,20..30): mod_base = OTHER; note = phosphorothioate
  modified_base 18^19: mod_base = OTHER; note = methylphosphonate
  modified_base order(1..2,4): mod_base = OTHER; note = m5u
  modified_base 5..6: mod_base = OTHER; note = m5c
  modified_base 17: mod_base = OTHER; note = 6-amino-5-nitro-3-yl-2(1H)-pyridone nucleobase
  modified_base 18: mod_base = OTHER; note = Inosine
  modified_base 1..30: mod_base = OTHER; note = B5
ttgtccttggtccaggnnagatgcaccttc

SEQ ID NO: 6 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..14,18,21..29): mod_base = OTHER; note = 2&apos;-O-methyl
  modified_base 19: mod_base = OTHER; note = 2&apos;-fluoro
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B6
tgtccttggtccaggnnagatgcaccttc

SEQ ID NO: 7 - DNA, 28 bp, synthetic construct
  source 1..28: mol_type = other DNA
  modified_base 15..16: mod_base = OTHER; note = DNA
  modified_base order(1..14,17..28): mod_base = OTHER; note = RNA
  modified_base order(1..4,14,19): mod_base = OTHER; note = 2&apos;-O-methoxyethyl
  modified_base order(5..13,17,20..28): mod_base = OTHER; note = 2&apos;-O-methyl
  modified_base 18: mod_base = OTHER; note = 2&apos;-fluoro
  modified_base order(5..14,15^16,18..28): mod_base = OTHER; note = phosphorothioate
  modified_base 16^17: mod_base = OTHER; note = methylphosphonate
  modified_base 2: mod_base = OTHER; note = m5u
  modified_base 3..4: mod_base = OTHER; note = m5c
  modified_base 15: mod_base = OTHER; note = 6-amino-5-nitro-3-yl-2(1H)-pyridone nucleobase
  modified_base 16: mod_base = OTHER; note = Inosine
  modified_base 1..28: mod_base = OTHER; note = B7
gtccttggtccaggnnagatgcaccttc

SEQ ID NO: 8 - DNA, 27 bp, synthetic construct
  source 1..27: mol_type = other DNA
  modified_base 14..15: mod_base = OTHER; note = DNA
  modified_base order(1..13,16..27): mod_base = OTHER; note = RNA
  modified_base order(1..3,13,18): mod_base = OTHER; note = 2&apos;-O-methoxyethyl
  modified_base order(4..12,16,19..27): mod_base = OTHER; note = 2&apos;-O-methyl
  modified_base 17: mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base 1: mod_base = OTHER; note = m5u
  modified_base 2..3: mod_base = OTHER; note = m5c
  modified_base 14: mod_base = OTHER; note = 6-amino-5-nitro-3-yl-2(1H)-pyridone nucleobase
  modified_base 15: mod_base = OTHER; note = Inosine
  modified_base 1..27: mod_base = OTHER; note = B8
tccttggtccaggnnagatgcaccttc

SEQ ID NO: 9 - DNA, 25 bp, synthetic construct
  source 1..25: mol_type = other DNA
  modified_base 12..13: mod_base = OTHER; note = DNA
  modified_base order(1..11,14..25): mod_base = OTHER; note = RNA
  modified_base order(1,11,16): mod_base = OTHER; note = 2&apos;-O-methoxyethyl
  modified_base order(2..10,14,17..25): mod_base = OTHER; note = 2&apos;-O-methyl
  modified_base 15: mod_base = OTHER; note = 2&apos;-fluoro
  modified_base order(2..11,12^13,15..25): mod_base = OTHER; note = phosphorothioate
  modified_base 13^14: mod_base = OTHER; note = methylphosphonate
  modified_base 1: mod_base = OTHER; note = m5c
  modified_base 12: mod_base = OTHER; note = 6-amino-5-nitro-3-yl-2(1H)-pyridone nucleobase
  modified_base 13: mod_base = OTHER; note = Inosine
  modified_base 1..25: mod_base = OTHER; note = B9
cttggtccaggnnagatgcaccttc

SEQ ID NO: 10 - DNA, 22 bp, synthetic construct
  source 1..22: mol_type = other DNA
  modified_base 9..10: mod_base = OTHER; note = DNA
  modified_base order(1..8,11..22): mod_base = OTHER; note = RNA
  modified_base order(1..7,11,14..22): mod_base = OTHER; note = 2&apos;-O-methyl
  modified_base order(8,13): mod_base = OTHER; note = 2&apos;-O-methoxyethyl
  modified_base 12: mod_base = OTHER; note = 2&apos;-fluoro
  modified_base order(1..8,9^10,12..22): mod_base = OTHER; note = phosphorothioate
  modified_base 10^11: mod_base = OTHER; note = methylphosphonate
  modified_base 9: mod_base = OTHER; note = 6-amino-5-nitro-3-yl-2(1H)-pyridone nucleobase
  modified_base 10: mod_base = OTHER; note = Inosine
  modified_base 1..22: mod_base = OTHER; note = B10
ggtccaggnnagatgcaccttc

SEQ ID NO: 11 - DNA, 27 bp, synthetic construct
  source 1..27: mol_type = other DNA
  modified_base 16..17: mod_base = OTHER; note = DNA
  modified_base order(1..15,18..27): mod_base = OTHER; note = RNA
  modified_base order(1..5,15,20): mod_base = OTHER; note = 2&apos;-O-methoxyethyl
  modified_base order(6..14,18,21..27): mod_base = OTHER; note = 2&apos;-O-methyl
  modified_base 19: mod_base = OTHER; note = 2&apos;-fluoro
  modified_base order(6..15,16^17,19..27): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..27: mod_base = OTHER; note = B11
tgtccttggtccaggnnagatgcacct

SEQ ID NO: 12 - DNA, 25 bp, synthetic construct
  source 1..25: mol_type = other DNA
  modified_base 16..17: mod_base = OTHER; note = DNA
  modified_base order(1..15,18..25): mod_base = OTHER; note = RNA
  modified_base order(1..5,15,20): mod_base = OTHER; note = 2&apos;-O-methoxyethyl
  modified_base order(6..14,18,21..25): mod_base = OTHER; note = 2&apos;-O-methyl
  modified_base 19: mod_base = OTHER; note = 2&apos;-fluoro
  modified_base order(6..15,16^17,19..25): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..25: mod_base = OTHER; note = B12
tgtccttggtccaggnnagatgcac

SEQ ID NO: 13 - DNA, 23 bp, synthetic construct
  source 1..23: mol_type = other DNA
  modified_base 16..17: mod_base = OTHER; note = DNA
  modified_base order(1..15,18..23): mod_base = OTHER; note = RNA
  modified_base order(1..5,15,20): mod_base = OTHER; note = 2&apos;-O-methoxyethyl
  modified_base order(6..14,18,21..23): mod_base = OTHER; note = 2&apos;-O-methyl
  modified_base 19: mod_base = OTHER; note = 2&apos;-fluoro
  modified_base order(6..15,16^17,19..23): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..23: mod_base = OTHER; note = B13
tgtccttggtccaggnnagatgc

SEQ ID NO: 14 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..14,19,21..29): mod_base = OTHER; note = 2&apos;-fluoro
  modified_base 18: mod_base = OTHER; note = 2&apos;-O-methyl
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B14
tgtccttggtccaggnnagatgcaccttc

SEQ ID NO: 15 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..8,18,27..29): mod_base = OTHER; note = 2&apos;-O-methyl
  modified_base order(9..14,19,21..26): mod_base = OTHER; note = 2&apos;-fluoro
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B15
tgtccttggtccaggnnagatgcaccttc

SEQ ID NO: 16 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6,8,10,12,14,19,21,23,25,27,29): mod_base = OTHER; note = 2&apos;-fluoro
  modified_base order(7,9,11,13,18,22,24,26,28): mod_base = OTHER; note = 2&apos;-O-methyl
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B16
tgtccttggtccaggnnagatgcaccttc

SEQ ID NO: 17 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..9,11,13,18,22,24,26..29): mod_base = OTHER; note = 2&apos;-O-methyl
  modified_base order(10,12,14,19,21,23,25): mod_base = OTHER; note = 2&apos;-fluoro
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B17
tgtccttggtccaggnnagatgcaccttc

SEQ ID NO: 18 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..8,18,27..29): mod_base = OTHER; note = 2&apos;-O-methyl
  modified_base order(9..14,19,21..26): mod_base = OTHER; note = 2&apos;-fluoro
  modified_base order(1^2,28^29): mod_base = OTHER; note = (1,3-dimethylimidazolidin-2-ylidene) phosphoramidate linkage
  modified_base order(6..15,16^17,19..28): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B18
tgtccttggtccaggnnagatgcaccttc

SEQ ID NO: 19 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6,8,10,12,14,19,21,23,25,27,29): mod_base = OTHER; note = 2&apos;-fluoro
  modified_base order(7,9,11,13,18,22,24,26,28): mod_base = OTHER; note = 2&apos;-O-methyl
  modified_base order(1^2,28^29): mod_base = OTHER; note = (1,3-dimethylimidazolidin-2-ylidene) phosphoramidate linkage
  modified_base order(6..15,16^17,19..28): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B19
tgtccttggtccaggnnagatgcaccttc

SEQ ID NO: 20 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..9,11,13,18,22,24,26..29): mod_base = OTHER; note = 2&apos;-O-methyl
  modified_base order(10,12,14,19,21,23,25): mod_base = OTHER; note = 2&apos;-fluoro
  modified_base order(1^2,28^29): mod_base = OTHER; note = (1,3-dimethylimidazolidin-2-ylidene) phosphoramidate linkage
  modified_base order(6..15,16^17,19..28): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B20
tgtccttggtccaggnnagatgcaccttc

SEQ ID NO: 21 - DNA, 25 bp, synthetic construct
  source 1..25: mol_type = other DNA
  modified_base 12..13: mod_base = OTHER; note = DNA
  modified_base order(1..11,14..25): mod_base = OTHER; note = RNA
  modified_base order(1,11,16): mod_base = OTHER; note = 2&apos;-O-methoxyethyl
  modified_base order(2..4,14,23..25): mod_base = OTHER; note = 2&apos;-O-methyl
  modified_base order(5..10,15,17..22): mod_base = OTHER; note = 2&apos;-fluoro
  modified_base order(2..11,12^13,15..25): mod_base = OTHER; note = phosphorothioate
  modified_base 13^14: mod_base = OTHER; note = methylphosphonate
  modified_base 1: mod_base = OTHER; note = m5c
  modified_base 12: mod_base = OTHER; note = 6-amino-5-nitro-3-yl-2(1H)-pyridone nucleobase
  modified_base 13: mod_base = OTHER; note = Inosine
  modified_base 1..25: mod_base = OTHER; note = B21
cttggtccaggnnagatgcaccttc

SEQ ID NO: 22 - DNA, 27 bp, synthetic construct
  source 1..27: mol_type = other DNA
  modified_base 14..15: mod_base = OTHER; note = DNA
  modified_base order(1..13,16..27): mod_base = OTHER; note = RNA
  modified_base order(1..3,13,18): mod_base = OTHER; note = 2&apos;-O-methoxyethyl
  modified_base order(4..6,16,25..27): mod_base = OTHER; note = 2&apos;-O-methyl
  modified_base order(7..12,17,19..24): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base 1: mod_base = OTHER; note = m5u
  modified_base 2..3: mod_base = OTHER; note = m5c
  modified_base 14: mod_base = OTHER; note = 6-amino-5-nitro-3-yl-2(1H)-pyridone nucleobase
  modified_base 15: mod_base = OTHER; note = Inosine
  modified_base 1..27: mod_base = OTHER; note = B22
tccttggtccaggnnagatgcaccttc

SEQ ID NO: 23 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..10,20,29..31): mod_base = OTHER; note = 2&apos;-O-methyl
  modified_base order(11..16,21,23..28): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B23

SEQ ID NO: 24 - DNA, 25 bp, synthetic construct
  source 1..25: mol_type = other DNA
  modified_base 12..13: mod_base = OTHER; note = DNA
  modified_base order(1..11,14..25): mod_base = OTHER; note = RNA
  modified_base order(1,11,16): mod_base = OTHER; note = 2&apos;-O-methoxyethyl
  modified_base order(2..4,14,23..25): mod_base = OTHER; note = 2&apos;-O-methyl
  modified_base order(5..10,15,17..22): mod_base = OTHER; note = 2&apos;-fluoro
  modified_base order(1^2,24^25): mod_base = OTHER; note = (1,3-dimethylimidazolidin-2-ylidene) phosphoramidate linkage
  modified_base order(2..11,12^13,15..24): mod_base = OTHER; note = phosphorothioate
  modified_base 13^14: mod_base = OTHER; note = methylphosphonate
  modified_base 1: mod_base = OTHER; note = m5c
  modified_base 12: mod_base = OTHER; note = 6-amino-5-nitro-3-yl-2(1H)-pyridone nucleobase
  modified_base 13: mod_base = OTHER; note = Inosine
  modified_base 1..25: mod_base = OTHER; note = B24
cttggtccaggnnagatgcaccttc

SEQ ID NO: 25 - DNA, 27 bp, synthetic construct
  source 1..27: mol_type = other DNA
  modified_base 14..15: mod_base = OTHER; note = DNA
  modified_base order(1..13,16..27): mod_base = OTHER; note = RNA
  modified_base order(1..3,13,18): mod_base = OTHER; note = 2&apos;-O-methoxyethyl
  modified_base order(4..6,16,25..27): mod_base = OTHER; note = 2&apos;-O-methyl
  modified_base order(7..12,17,19..24): mod_base = OTHER; note = 2&apos;-fluoro
  modified_base order(1^2,26^27): mod_base = OTHER; note = (1,3-dimethylimidazolidin-2-ylidene) phosphoramidate linkage
  modified_base order(4..13,14^15,17..26): mod_base = OTHER; note = phosphorothioate
  modified_base 15^16: mod_base = OTHER; note = methylphosphonate
  modified_base 1: mod_base = OTHER; note = m5u
  modified_base 2..3: mod_base = OTHER; note = m5c
  modified_base 14: mod_base = OTHER; note = 6-amino-5-nitro-3-yl-2(1H)-pyridone nucleobase
  modified_base 15: mod_base = OTHER; note = Inosine
  modified_base 1..27: mod_base = OTHER; note = B25
tccttggtccaggnnagatgcaccttc

SEQ ID NO: 26 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..10,20,29..31): mod_base = OTHER; note = 2&apos;-O-methyl
  modified_base order(11..16,21,23..28): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B26

SEQ ID NO: 27 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..8,18,21..29): mod_base = OTHER; note = 2&apos;-O-methyl
  modified_base order(9..14,19): mod_base = OTHER; note = 2&apos;-fluoro
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B27
tgtccttggtccaggnnagatgcaccttc

SEQ ID NO: 28 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..14,18,27..29): mod_base = OTHER; note = 2&apos;-O-methyl
  modified_base order(19,21..26): mod_base = OTHER; note = 2&apos;-fluoro
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B28
tgtccttggtccaggnnagatgcaccttc

SEQ ID NO: 29 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..8,18,27..29): mod_base = OTHER; note = 2&apos;-O-methyl
  modified_base order(9..14,19,21..26): mod_base = OTHER; note = 2&apos;-fluoro
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base order(14,17): mod_base = OTHER; note = Inosine
  modified_base 16: mod_base = OTHER; note = 6-amino-5-nitro-3-yl-2(1H)-pyridone nucleobase
  modified_base 1..29: mod_base = OTHER; note = B29
tgtccttggtccangnnagatgcaccttc

SEQ ID NO: 30 - DNA, 29 bp, synthetic construct
  source 1..29: mol_type = other DNA
  modified_base 11..12: mod_base = OTHER; note = DNA
  modified_base order(1..10,13..29): mod_base = OTHER; note = RNA
  modified_base order(1..3,13,22..24): mod_base = OTHER; note = 2&apos;-O-methyl
  modified_base order(4..9,14,16..21): mod_base = OTHER; note = 2&apos;-fluoro
  modified_base order(10,15,25..29): mod_base = OTHER; note = 2&apos;-O-methoxyethyl
  modified_base order(1..10,11^12,14..24): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base 12: mod_base = OTHER; note = Inosine
  modified_base 25: mod_base = OTHER; note = m5u
  modified_base 26..27: mod_base = OTHER; note = m5c
  modified_base 1..29: mod_base = OTHER; note = B30
ttggtccaggnnagatgcaccttctccgg

SEQ ID NO: 31 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..8,18,27..29): mod_base = OTHER; note = 2&apos;-O-methyl
  modified_base order(9..14,19,21..26): mod_base = OTHER; note = 2&apos;-fluoro
  modified_base order(1^2,23^24,28^29): mod_base = OTHER; note = (1,3-dimethylimidazolidin-2-ylidene) phosphoramidate linkage
  modified_base order(6..15,16^17,19..23,24..28): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B31
tgtccttggtccaggnnagatgcaccttc

SEQ ID NO: 32 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..8,18,27..29): mod_base = OTHER; note = 2&apos;-O-methyl
  modified_base order(9..14,19,21..26): mod_base = OTHER; note = 2&apos;-fluoro
  modified_base order(1^2,11^12,28^29): mod_base = OTHER; note = (1,3-dimethylimidazolidin-2-ylidene) phosphoramidate linkage
  modified_base order(6..11,12..15,16^17,19..28): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B32
tgtccttggtccaggnnagatgcaccttc

SEQ ID NO: 33 - DNA, 32 bp, synthetic construct; identical to SEQ ID NO: 3
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..11,21,30..32): mod_base = OTHER; note = 2&apos;-O-methyl
  modified_base order(12..17,22,24..29): mod_base = OTHER; note = 2&apos;-fluoro
  modified_base order(9..18,19^20,22..32): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..32: mod_base = OTHER; note = B33

SEQ ID NO: 34 - DNA, 32 bp, synthetic construct; identical to SEQ ID NO: 3
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..11,21,30..32): mod_base = OTHER; note = 2&apos;-O-methyl
  modified_base order(12..17,22,24..29): mod_base = OTHER; note = 2&apos;-fluoro
  modified_base order(1^2,31^32): mod_base = OTHER; note = (1,3-dimethylimidazolidin-2-ylidene) phosphoramidate linkage
  modified_base order(9..18,19^20,22..31): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..32: mod_base = OTHER; note = B34

SEQ ID NO: 35 - DNA, 32 bp, synthetic construct; identical to SEQ ID NO: 3
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..11,21,24..32): mod_base = OTHER; note = 2&apos;-O-methyl
  modified_base order(12..17,22): mod_base = OTHER; note = 2&apos;-fluoro
  modified_base order(9..18,19^20,22..32): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..32: mod_base = OTHER; note = B35

SEQ ID NO: 36 - DNA, 32 bp, synthetic construct; identical to SEQ ID NO: 3
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..17,21,30..32): mod_base = OTHER; note = 2&apos;-O-methyl
  modified_base order(22,24..29): mod_base = OTHER; note = 2&apos;-fluoro
  modified_base order(9..18,19^20,22..32): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..32: mod_base = OTHER; note = B36

SEQ ID NO: 37 - DNA, 32 bp, synthetic construct
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..11,21,30..32): mod_base = OTHER; note = 2&apos;-O-methyl
  modified_base order(12..17,22,24..29): mod_base = OTHER; note = 2&apos;-fluoro
  modified_base order(9..18,19^20,22..32): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base order(17,20): mod_base = OTHER; note = Inosine
  modified_base 19: mod_base = OTHER; note = 6-amino-5-nitro-3-yl-2(1H)-pyridone nucleobase
  modified_base 1..32: mod_base = OTHER; note = B37
acttgtccttggtccangnnagatgcaccttc

SEQ ID NO: 38 - DNA, 29 bp, synthetic construct
  source 1..29: mol_type = other DNA
  modified_base 11..12: mod_base = OTHER; note = DNA
  modified_base order(1..10,13..29): mod_base = OTHER; note = RNA
  modified_base order(1..3,13,22..24): mod_base = OTHER; note = 2&apos;-O-methyl
  modified_base order(4..9,14,16..21): mod_base = OTHER; note = 2&apos;-fluoro
  modified_base order(10,15,25..29): mod_base = OTHER; note = 2&apos;-O-methoxyethyl
  modified_base order(1..10,11^12,14..24): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base 12: mod_base = OTHER; note = Inosine
  modified_base 25: mod_base = OTHER; note = m5u
  modified_base 26..27: mod_base = OTHER; note = m5c
  modified_base 1..29: mod_base = OTHER; note = B38
ttggtccaggnnagatgcaccttctccgg

SEQ ID NO: 39 - DNA, 32 bp, synthetic construct; identical to SEQ ID NO: 3
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..11,21,30..32): mod_base = OTHER; note = 2&apos;-O-methyl
  modified_base order(12..17,22,24..29): mod_base = OTHER; note = 2&apos;-fluoro
  modified_base order(1^2,26^27,31^32): mod_base = OTHER; note = (1,3-dimethylimidazolidin-2-ylidene) phosphoramidate linkage
  modified_base order(9..18,19^20,22..26,27..31): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..32: mod_base = OTHER; note = B39

SEQ ID NO: 40 - DNA, 32 bp, synthetic construct; identical to SEQ ID NO: 3
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..11,21,30..32): mod_base = OTHER; note = 2&apos;-O-methyl
  modified_base order(12..17,22,24..29): mod_base = OTHER; note = 2&apos;-fluoro
  modified_base order(1^2,15^16,31^32): mod_base = OTHER; note = (1,3-dimethylimidazolidin-2-ylidene) phosphoramidate linkage
  modified_base order(9..15,16..18,19^20,22..31): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..32: mod_base = OTHER; note = B40

SEQ ID NO: 41 - DNA, 26 bp, synthetic construct
  source 1..26: mol_type = other DNA
  modified_base 18..19: mod_base = OTHER; note = DNA
  modified_base order(1..17,20..26): mod_base = OTHER; note = RNA
  modified_base order(1..7,17,22): mod_base = OTHER; note = 2&apos;-O-methoxyethyl
  modified_base order(8..10,20): mod_base = OTHER; note = 2&apos;-O-methyl
  modified_base order(11..16,21,23..26): mod_base = OTHER; note = 2&apos;-fluoro
  modified_base order(8..17,18^19,21..26): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..26: mod_base = OTHER; note = B41
cttgtccttggtccaggnnagatgca

SEQ ID NO: 42 - DNA, 27 bp, synthetic construct
  source 1..27: mol_type = other DNA
  modified_base 18..19: mod_base = OTHER; note = DNA
  modified_base order(1..17,20..27): mod_base = OTHER; note = RNA
  modified_base order(1..7,17,22): mod_base = OTHER; note = 2&apos;-O-methoxyethyl
  modified_base order(8..10,20): mod_base = OTHER; note = 2&apos;-O-methyl
  modified_base order(11..16,21,23..27): mod_base = OTHER; note = 2&apos;-fluoro
  modified_base order(8..17,18^19,21..27): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..27: mod_base = OTHER; note = B42
cttgtccttggtccaggnnagatgcac

SEQ ID NO: 43 - DNA, 28 bp, synthetic construct
  source 1..28: mol_type = other DNA
  modified_base 18..19: mod_base = OTHER; note = DNA
  modified_base order(1..17,20..28): mod_base = OTHER; note = RNA
  modified_base order(1..7,17,22): mod_base = OTHER; note = 2&apos;-O-methoxyethyl
  modified_base order(8..10,20): mod_base = OTHER; note = 2&apos;-O-methyl
  modified_base order(11..16,21,23..28): mod_base = OTHER; note = 2&apos;-fluoro
  modified_base order(8..17,18^19,21..28): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..28: mod_base = OTHER; note = B43
cttgtccttggtccaggnnagatgcacc

SEQ ID NO: 44 - DNA, 29 bp, synthetic construct
  source 1..29: mol_type = other DNA
  modified_base 18..19: mod_base = OTHER; note = DNA
  modified_base order(1..17,20..29): mod_base = OTHER; note = RNA
  modified_base order(1..7,17,22): mod_base = OTHER; note = 2&apos;-O-methoxyethyl
  modified_base order(8..10,20,29): mod_base = OTHER; note = 2&apos;-O-methyl
  modified_base order(11..16,21,23..28): mod_base = OTHER; note = 2&apos;-fluoro
  modified_base order(8..17,18^19,21..29): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..29: mod_base = OTHER; note = B44
cttgtccttggtccaggnnagatgcacct

SEQ ID NO: 45 - DNA, 30 bp, synthetic construct
  source 1..30: mol_type = other DNA
  modified_base 18..19: mod_base = OTHER; note = DNA
  modified_base order(1..17,20..30): mod_base = OTHER; note = RNA
  modified_base order(1..7,17,22): mod_base = OTHER; note = 2&apos;-O-methoxyethyl
  modified_base order(8..10,20,29..30): mod_base = OTHER; note = 2&apos;-O-methyl
  modified_base order(11..16,21,23..28): mod_base = OTHER; note = 2&apos;-fluoro
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..30: mod_base = OTHER; note = B45
cttgtccttggtccaggnnagatgcacctt

SEQ ID NO: 46 - DNA, 31 bp, synthetic construct
  source 1..31: mol_type = other DNA
  modified_base 21..22: mod_base = OTHER; note = DNA
  modified_base order(1..20,23..31): mod_base = OTHER; note = RNA
  modified_base order(1..10,20,25): mod_base = OTHER; note = 2&apos;-O-methoxyethyl
  modified_base order(11..13,23): mod_base = OTHER; note = 2&apos;-O-methyl
  modified_base order(14..19,24,26..31): mod_base = OTHER; note = 2&apos;-fluoro
  modified_base order(11..20,21^22,24..31): mod_base = OTHER; note = phosphorothioate
  modified_base 22^23: mod_base = OTHER; note = methylphosphonate
  modified_base order(1,4,9..10): mod_base = OTHER; note = m5c
  modified_base order(5..6,8): mod_base = OTHER; note = m5u
  modified_base 21: mod_base = OTHER; note = 6-amino-5-nitro-3-yl-2(1H)-pyridone nucleobase
  modified_base 22: mod_base = OTHER; note = Inosine
  modified_base 1..31: mod_base = OTHER; note = B46
cgacttgtccttggtccaggnnagatgcacc

SEQ ID NO: 47 - DNA, 30 bp, synthetic construct
  source 1..30: mol_type = other DNA
  modified_base 20..21: mod_base = OTHER; note = DNA
  modified_base order(1..19,22..30): mod_base = OTHER; note = RNA
  modified_base order(1..9,19,24): mod_base = OTHER; note = 2&apos;-O-methoxyethyl
  modified_base order(10..12,22): mod_base = OTHER; note = 2&apos;-O-methyl
  modified_base order(13..18,23,25..30): mod_base = OTHER; note = 2&apos;-fluoro
  modified_base order(10..19,20^21,23..30): mod_base = OTHER; note = phosphorothioate
  modified_base 21^22: mod_base = OTHER; note = methylphosphonate
  modified_base order(3,8..9): mod_base = OTHER; note = m5c
  modified_base order(4..5,7): mod_base = OTHER; note = m5u
  modified_base 20: mod_base = OTHER; note = 6-amino-5-nitro-3-yl-2(1H)-pyridone nucleobase
  modified_base 21: mod_base = OTHER; note = Inosine
  modified_base 1..30: mod_base = OTHER; note = B47
gacttgtccttggtccaggnnagatgcacc

SEQ ID NO: 48 - DNA, 29 bp, synthetic construct
  source 1..29: mol_type = other DNA
  modified_base 19..20: mod_base = OTHER; note = DNA
  modified_base order(1..18,21..29): mod_base = OTHER; note = RNA
  modified_base order(1..8,18,23): mod_base = OTHER; note = 2&apos;-O-methoxyethyl
  modified_base order(9..11,21): mod_base = OTHER; note = 2&apos;-O-methyl
  modified_base order(12..17,22,24..29): mod_base = OTHER; note = 2&apos;-fluoro
  modified_base order(9..18,19^20,22..29): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..29: mod_base = OTHER; note = B48
acttgtccttggtccaggnnagatgcacc

SEQ ID NO: 49 - DNA, 31 bp, synthetic construct
  source 1..31: mol_type = other DNA
  modified_base 20..21: mod_base = OTHER; note = DNA
  modified_base order(1..19,22..31): mod_base = OTHER; note = RNA
  modified_base order(1..9,19,24): mod_base = OTHER; note = 2&apos;-O-methoxyethyl
  modified_base order(10..12,22,31): mod_base = OTHER; note = 2&apos;-O-methyl
  modified_base order(13..18,23,25..30): mod_base = OTHER; note = 2&apos;-fluoro
  modified_base order(10..19,20^21,23..31): mod_base = OTHER; note = phosphorothioate
  modified_base 21^22: mod_base = OTHER; note = methylphosphonate
  modified_base order(3,8..9): mod_base = OTHER; note = m5c
  modified_base order(4..5,7): mod_base = OTHER; note = m5u
  modified_base 20: mod_base = OTHER; note = 6-amino-5-nitro-3-yl-2(1H)-pyridone nucleobase
  modified_base 21: mod_base = OTHER; note = Inosine
  modified_base 1..31: mod_base = OTHER; note = B49
gacttgtccttggtccaggnnagatgcacct

SEQ ID NO: 50 - DNA, 30 bp, synthetic construct
  source 1..30: mol_type = other DNA
  modified_base 19..20: mod_base = OTHER; note = DNA
  modified_base order(1..18,21..30): mod_base = OTHER; note = RNA
  modified_base order(1..8,18,23): mod_base = OTHER; note = 2&apos;-O-methoxyethyl
  modified_base order(9..11,21,30): mod_base = OTHER; note = 2&apos;-O-methyl
  modified_base order(12..17,22,24..29): mod_base = OTHER; note = 2&apos;-fluoro
  modified_base order(9..18,19^20,22..30): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..30: mod_base = OTHER; note = B50
acttgtccttggtccaggnnagatgcacct

SEQ ID NO: 51 - DNA, 28 bp, synthetic construct
  source 1..28: mol_type = other DNA
  modified_base 17..18: mod_base = OTHER; note = DNA
  modified_base order(1..16,19..28): mod_base = OTHER; note = RNA
  modified_base order(1..6,16,21): mod_base = OTHER; note = 2&apos;-O-methoxyethyl
  modified_base order(7..9,19,28): mod_base = OTHER; note = 2&apos;-O-methyl
  modified_base order(10..15,20,22..27): mod_base = OTHER; note = 2&apos;-fluoro
  modified_base order(7..16,17^18,20..28): mod_base = OTHER; note = phosphorothioate
  modified_base 18^19: mod_base = OTHER; note = methylphosphonate
  modified_base order(1..2,4): mod_base = OTHER; note = m5u
  modified_base 5..6: mod_base = OTHER; note = m5c
  modified_base 17: mod_base = OTHER; note = 6-amino-5-nitro-3-yl-2(1H)-pyridone nucleobase
  modified_base 18: mod_base = OTHER; note = Inosine
  modified_base 1..28: mod_base = OTHER; note = B51
ttgtccttggtccaggnnagatgcacct

SEQ ID NO: 52 - DNA, 27 bp, synthetic construct
  source 1..27: mol_type = other DNA
  modified_base 14..15: mod_base = OTHER; note = DNA
  modified_base order(1..13,16..27): mod_base = OTHER; note = RNA
  modified_base order(1..3,13,18): mod_base = OTHER; note = 2&apos;-O-methoxyethyl
  modified_base order(4..12,16,25..27): mod_base = OTHER; note = 2&apos;-O-methyl
  modified_base order(17,19..24): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base 1: mod_base = OTHER; note = m5u
  modified_base 2..3: mod_base = OTHER; note = m5c
  modified_base 14: mod_base = OTHER; note = 6-amino-5-nitro-3-yl-2(1H)-pyridone nucleobase
  modified_base 15: mod_base = OTHER; note = Inosine
  modified_base 1..27: mod_base = OTHER; note = B52
tccttggtccaggnnagatgcaccttc

SEQ ID NO: 53 - DNA, 27 bp, synthetic construct
  source 1..27: mol_type = other DNA
  modified_base 14..15: mod_base = OTHER; note = DNA
  modified_base order(1..13,16..27): mod_base = OTHER; note = RNA
  modified_base order(1..3,13,18): mod_base = OTHER; note = 2&apos;-O-methoxyethyl
  modified_base order(4..6,8,10,12,16,25..27): mod_base = OTHER; note = 2&apos;-O-methyl
  modified_base order(7,9,11,17,19..24): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base 1: mod_base = OTHER; note = m5u
  modified_base 2..3: mod_base = OTHER; note = m5c
  modified_base 14: mod_base = OTHER; note = 6-amino-5-nitro-3-yl-2(1H)-pyridone nucleobase
  modified_base 15: mod_base = OTHER; note = Inosine
  modified_base 1..27: mod_base = OTHER; note = B53
tccttggtccaggnnagatgcaccttc

SEQ ID NO: 54 - DNA, 27 bp, synthetic construct
  source 1..27: mol_type = other DNA
  modified_base 14..15: mod_base = OTHER; note = DNA
  modified_base order(1..13,16..27): mod_base = OTHER; note = RNA
  modified_base order(1..3,13,18): mod_base = OTHER; note = 2&apos;-O-methoxyethyl
  modified_base order(4..5,7,9,11,16,25..27): mod_base = OTHER; note = 2&apos;-O-methyl
  modified_base order(6,8,10,12,17,19..24): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base 1: mod_base = OTHER; note = m5u
  modified_base 2..3: mod_base = OTHER; note = m5c
  modified_base 14: mod_base = OTHER; note = 6-amino-5-nitro-3-yl-2(1H)-pyridone nucleobase
  modified_base 15: mod_base = OTHER; note = Inosine
  modified_base 1..27: mod_base = OTHER; note = B54
tccttggtccaggnnagatgcaccttc

SEQ ID NO: 55 - DNA, 27 bp, synthetic construct
  source 1..27: mol_type = other DNA
  modified_base 17..18: mod_base = OTHER; note = DNA
  modified_base order(1..16,19..27): mod_base = OTHER; note = RNA
  modified_base order(1..6,16,21): mod_base = OTHER; note = 2&apos;-O-methoxyethyl
  modified_base order(7..9,19): mod_base = OTHER; note = 2&apos;-O-methyl
  modified_base order(10..15,20,22..27): mod_base = OTHER; note = 2&apos;-fluoro
  modified_base order(7..16,17^18,20..27): mod_base = OTHER; note = phosphorothioate
  modified_base 18^19: mod_base = OTHER; note = methylphosphonate
  modified_base order(1..2,4): mod_base = OTHER; note = m5u
  modified_base 5..6: mod_base = OTHER; note = m5c
  modified_base 17: mod_base = OTHER; note = 6-amino-5-nitro-3-yl-2(1H)-pyridone nucleobase
  modified_base 18: mod_base = OTHER; note = Inosine
  modified_base 1..27: mod_base = OTHER; note = B55
ttgtccttggtccaggnnagatgcacc

SEQ ID NO: 56 - DNA, 27 bp, synthetic construct
  source 1..27: mol_type = other DNA
  modified_base 17..18: mod_base = OTHER; note = DNA
  modified_base order(1..16,19..27): mod_base = OTHER; note = RNA
  modified_base order(1..6,16,21): mod_base = OTHER; note = 2&apos;-O-methoxyethyl
  modified_base order(7..15,19): mod_base = OTHER; note = 2&apos;-O-methyl
  modified_base order(20,22..27): mod_base = OTHER; note = 2&apos;-fluoro
  modified_base order(7..16,17^18,20..27): mod_base = OTHER; note = phosphorothioate
  modified_base 18^19: mod_base = OTHER; note = methylphosphonate
  modified_base order(1..2,4): mod_base = OTHER; note = m5u
  modified_base 5..6: mod_base = OTHER; note = m5c
  modified_base 17: mod_base = OTHER; note = 6-amino-5-nitro-3-yl-2(1H)-pyridone nucleobase
  modified_base 18: mod_base = OTHER; note = Inosine
  modified_base 1..27: mod_base = OTHER; note = B56
ttgtccttggtccaggnnagatgcacc

SEQ ID NO: 57 - DNA, 27 bp, synthetic construct
  source 1..27: mol_type = other DNA
  modified_base 17..18: mod_base = OTHER; note = DNA
  modified_base order(1..16,19..27): mod_base = OTHER; note = RNA
  modified_base order(1..6,16,21): mod_base = OTHER; note = 2&apos;-O-methoxyethyl
  modified_base order(7..9,11,15,19): mod_base = OTHER; note = 2&apos;-O-methyl
  modified_base order(10,12..14,20,22..27): mod_base = OTHER; note = 2&apos;-fluoro
  modified_base order(7..16,17^18,20..27): mod_base = OTHER; note = phosphorothioate
  modified_base 18^19: mod_base = OTHER; note = methylphosphonate
  modified_base order(1..2,4): mod_base = OTHER; note = m5u
  modified_base 5..6: mod_base = OTHER; note = m5c
  modified_base 17: mod_base = OTHER; note = 6-amino-5-nitro-3-yl-2(1H)-pyridone nucleobase
  modified_base 18: mod_base = OTHER; note = Inosine
  modified_base 1..27: mod_base = OTHER; note = B57
ttgtccttggtccaggnnagatgcacc

SEQ ID NO: 58 - DNA, 27 bp, synthetic construct
  source 1..27: mol_type = other DNA
  modified_base 17..18: mod_base = OTHER; note = DNA
  modified_base order(1..16,19..27): mod_base = OTHER; note = RNA
  modified_base order(1..6,16,21): mod_base = OTHER; note = 2&apos;-O-methoxyethyl
  modified_base order(7..8,10,12,14,19): mod_base = OTHER; note = 2&apos;-O-methyl
  modified_base order(9,11,13,15,20,22..27): mod_base = OTHER; note = 2&apos;-fluoro
  modified_base order(7..16,17^18,20..27): mod_base = OTHER; note = phosphorothioate
  modified_base 18^19: mod_base = OTHER; note = methylphosphonate
  modified_base order(1..2,4): mod_base = OTHER; note = m5u
  modified_base 5..6: mod_base = OTHER; note = m5c
  modified_base 17: mod_base = OTHER; note = 6-amino-5-nitro-3-yl-2(1H)-pyridone nucleobase
  modified_base 18: mod_base = OTHER; note = Inosine
  modified_base 1..27: mod_base = OTHER; note = B58
ttgtccttggtccaggnnagatgcacc

SEQ ID NO: 59 - DNA, 27 bp, synthetic construct
  source 1..27: mol_type = other DNA
  modified_base 14..15: mod_base = OTHER; note = DNA
  modified_base order(1..13,16..27): mod_base = OTHER; note = RNA
  modified_base order(1..3,13,18): mod_base = OTHER; note = 2&apos;-O-methoxyethyl
  modified_base order(4..6,16,25..27): mod_base = OTHER; note = 2&apos;-O-methyl
  modified_base order(7..12,17,19..24): mod_base = OTHER; note = 2&apos;-fluoro
  modified_base order(1..3,4..13,14^15,17..27): mod_base = OTHER; note = phosphorothioate
  modified_base 15^16: mod_base = OTHER; note = methylphosphonate
  modified_base 1: mod_base = OTHER; note = m5u
  modified_base 2..3: mod_base = OTHER; note = m5c
  modified_base 14: mod_base = OTHER; note = 6-amino-5-nitro-3-yl-2(1H)-pyridone nucleobase
  modified_base 15: mod_base = OTHER; note = Inosine
  modified_base 1..27: mod_base = OTHER; note = B59
tccttggtccaggnnagatgcaccttc

SEQ ID NO: 60 - DNA, 27 bp, synthetic construct
  source 1..27: mol_type = other DNA
  modified_base 14..15: mod_base = OTHER; note = DNA
  modified_base order(1..13,16..27): mod_base = OTHER; note = RNA
  modified_base order(1..6,16,25..27): mod_base = OTHER; note = 2&apos;-O-methyl
  modified_base order(7..12,17,19..24): mod_base = OTHER; note = 2&apos;-fluoro
  modified_base order(13,18): mod_base = OTHER; note = 2&apos;-O-methoxyethyl
  modified_base order(1..13,14^15,17..27): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 15: mod_base = OTHER; note = Inosine
  modified_base 1..27: mod_base = OTHER; note = B60
tccttggtccaggnnagatgcaccttc

SEQ ID NO: 61 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..10,20,29..31): mod_base = OTHER; note = 2&apos;-O-methyl
  modified_base order(11..16,21,23..28): mod_base = OTHER; note = 2&apos;-fluoro
  modified_base order(1..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B61

SEQ ID NO: 62 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..10,20,29..31): mod_base = OTHER; note = 2&apos;-O-methyl
  modified_base order(11..16,21,23..28): mod_base = OTHER; note = 2&apos;-fluoro
  modified_base order(17,22): mod_base = OTHER; note = 2&apos;-O-methoxyethyl
  modified_base order(1..17,18^19,21..31): mod_base = OTHER; note = phosphorothioate
  modified_base 19^20: mod_base = OTHER; note = methylphosphonate
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B62

SEQ ID NO: 63 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..10,20,29..31): mod_base = OTHER; note = 2&apos;-O-methyl
  modified_base order(11..16,21,23..28): mod_base = OTHER; note = 2&apos;-fluoro
  modified_base order(17,22): mod_base = OTHER; note = 2&apos;-O-methoxyethyl
  modified_base order(1^2,30^31): mod_base = OTHER; note = (1,3-dimethylimidazolidin-2-ylidene) phosphoramidate linkage
  modified_base order(2..17,18^19,21..30): mod_base = OTHER; note = phosphorothioate
  modified_base 19^20: mod_base = OTHER; note = methylphosphonate
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B63

SEQ ID NO: 64 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B64

SEQ ID NO: 65 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(1..7,8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B65

SEQ ID NO: 66 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B66

SEQ ID NO: 67 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B67

SEQ ID NO: 68 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B68

SEQ ID NO: 69 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,29..31): mod_base = OTHER; note = 2&apos;-O-methyl
  modified_base order(21,23..28): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B69

SEQ ID NO: 70 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,29..31): mod_base = OTHER; note = 2&apos;-O-methyl
  modified_base order(21,23..28): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B70

SEQ ID NO: 71 - DNA, 29 bp, synthetic construct
  source 1..29: mol_type = other DNA
  modified_base 16..17: mod_base = OTHER; note = DNA
  modified_base order(1..15,18..29): mod_base = OTHER; note = RNA
  modified_base order(1..5,15,20): mod_base = OTHER; note = 2&apos;-O-methoxyethyl
  modified_base order(6..9,11,13,18,27..29): mod_base = OTHER; note = 2&apos;-O-methyl
  modified_base order(10,12,14,19,21..26): mod_base = OTHER; note = 2&apos;-fluoro
  modified_base order(6..15,16^17,19..29): mod_base = OTHER; note = phosphorothioate
  modified_base 17^18: mod_base = OTHER; note = methylphosphonate
  modified_base order(1,3): mod_base = OTHER; note = m5u
  modified_base 4..5: mod_base = OTHER; note = m5c
  modified_base 16: mod_base = OTHER; note = 6-amino-5-nitro-3-yl-2(1H)-pyridone nucleobase
  modified_base 17: mod_base = OTHER; note = Inosine
  modified_base 1..29: mod_base = OTHER; note = B71
tgtccttggtccaggnnagatgcaccttc

SEQ ID NO: 72 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B72

SEQ ID NO: 73 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B73

SEQ ID NO: 74 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B74

SEQ ID NO: 75 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(1..7,8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B75

SEQ ID NO: 76 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..11,13,15,20,29..31): mod_base = OTHER; note = 2&apos;-O-methyl
  modified_base order(12,14,16,21,23..28): mod_base = OTHER; note = 2&apos;-fluoro
  modified_base order(17,22): mod_base = OTHER; note = 2&apos;-O-methoxyethyl
  modified_base order(1..17,18^19,21..31): mod_base = OTHER; note = phosphorothioate
  modified_base 19^20: mod_base = OTHER; note = methylphosphonate
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B76

SEQ ID NO: 77 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(2..7,8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B77

SEQ ID NO: 78 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..11,13,15,20,29..31): mod_base = OTHER; note = 2&apos;-O-methyl
  modified_base order(12,14,16,21,23..28): mod_base = OTHER; note = 2&apos;-fluoro
  modified_base order(17,22): mod_base = OTHER; note = 2&apos;-O-methoxyethyl
  modified_base order(1^2,30^31): mod_base = OTHER; note = (1,3-dimethylimidazolidin-2-ylidene) phosphoramidate linkage
  modified_base order(2..17,18^19,21..30): mod_base = OTHER; note = phosphorothioate
  modified_base 19^20: mod_base = OTHER; note = methylphosphonate
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B78

SEQ ID NO: 79 - RNA, 38 nt, synthetic construct
  source 1..38: mol_type = other RNA
  modified_base order(1..24,27..38): mod_base = OTHER; note = RNA
  modified_base 25..26: mod_base = OTHER; note = DNA
  modified_base 25: mod_base = OTHER; note = 6-amino-5-nitro-3-yl-2(1H)-pyridone nucleobase
  modified_base 26: mod_base = OTHER; note = inosine
ccaccgacttgtccttggtccaggnnagatgcaccttc

SEQ ID NO: 80 - RNA, 34 nt, synthetic construct
  source 1..34: mol_type = other RNA
  modified_base order(1..20,23..34): mod_base = OTHER; note = RNA
  modified_base 21..22: mod_base = OTHER; note = DNA
  modified_base 22: mod_base = OTHER; note = inosine
  modified_base 21: mod_base = OTHER; note = 6-amino-5-nitro-3-yl-2(1H)-pyridone nucleobase
cgacttgtccttggtccaggnnagatgcaccttc

SEQ ID NO: 81 - RNA, 32 nt, synthetic construct
  source 1..32: mol_type = other RNA
  modified_base order(1..18,21..32): mod_base = OTHER; note = RNA
  modified_base 19..20: mod_base = OTHER; note = DNA
  modified_base 20: mod_base = OTHER; note = inosine
  modified_base 19: mod_base = OTHER; note = 6-amino-5-nitro-3-yl-2(1H)-pyridone nucleobase
acttgtccttggtccaggnnagatgcaccttc

SEQ ID NO: 82 - RNA, 31 nt, synthetic construct
  source 1..31: mol_type = other RNA
  modified_base order(1..17,20..31): mod_base = OTHER; note = RNA
  modified_base 18..19: mod_base = OTHER; note = DNA
  modified_base 19: mod_base = OTHER; note = inosine
  modified_base 18: mod_base = OTHER; note = 6-amino-5-nitro-3-yl-2(1H)-pyridone nucleobase
cttgtccttggtccaggnnagatgcaccttc

SEQ ID NO: 83 - RNA, 30 nt, synthetic construct
  source 1..30: mol_type = other RNA
  modified_base order(1..16,19..30): mod_base = OTHER; note = RNA
  modified_base 17..18: mod_base = OTHER; note = DNA
  modified_base 17: mod_base = OTHER; note = 6-amino-5-nitro-3-yl-2(1H)-pyridone nucleobase
  modified_base 18: mod_base = OTHER; note = inosine
ttgtccttggtccaggnnagatgcaccttc

SEQ ID NO: 84 - RNA, 29 nt, synthetic construct
  source 1..29: mol_type = other RNA
  modified_base order(1..15,18..29): mod_base = OTHER; note = RNA
  modified_base 16..17: mod_base = OTHER; note = DNA
  modified_base 17: mod_base = OTHER; note = inosine
  modified_base 16: mod_base = OTHER; note = 6-amino-5-nitro-3-yl-2(1H)-pyridone nucleobase
tgtccttggtccaggnnagatgcaccttc

SEQ ID NO: 85 - RNA, 28 nt, synthetic construct
  source 1..28: mol_type = other RNA
  modified_base order(1..14,17..28): mod_base = OTHER; note = RNA
  modified_base 15..16: mod_base = OTHER; note = DNA
  modified_base 16: mod_base = OTHER; note = inosine
  modified_base 15: mod_base = OTHER; note = 6-amino-5-nitro-3-yl-2(1H)-pyridone nucleobase
gtccttggtccaggnnagatgcaccttc

SEQ ID NO: 86 - RNA, 27 nt, synthetic construct
  source 1..27: mol_type = other RNA
  modified_base order(1..13,16..27): mod_base = OTHER; note = RNA
  modified_base 14..15: mod_base = OTHER; note = DNA
  modified_base 15: mod_base = OTHER; note = inosine
  modified_base 14: mod_base = OTHER; note = 6-amino-5-nitro-3-yl-2(1H)-pyridone nucleobase
tccttggtccaggnnagatgcaccttc

SEQ ID NO: 87 - RNA, 25 nt, synthetic construct
  source 1..25: mol_type = other RNA
  modified_base order(1..11,14..25): mod_base = OTHER; note = RNA
  modified_base 13: mod_base = OTHER; note = inosine
  modified_base 12..13: mod_base = OTHER; note = DNA
  modified_base 12: mod_base = OTHER; note = 6-amino-5-nitro-3-yl-2(1H)-pyridone nucleobase
cttggtccaggnnagatgcaccttc

SEQ ID NO: 88 - RNA, 22 nt, synthetic construct
  source 1..22: mol_type = other RNA
  modified_base order(1..8,11..22): mod_base = OTHER; note = RNA
  modified_base 9..10: mod_base = OTHER; note = DNA
  modified_base 10: mod_base = OTHER; note = inosine
  modified_base 9: mod_base = OTHER; note = 6-amino-5-nitro-3-yl-2(1H)-pyridone nucleobase
ggtccaggnnagatgcaccttc

SEQ ID NO: 89 - RNA, 27 nt, synthetic construct
  source 1..27: mol_type = other RNA
  modified_base order(1..15,18..27): mod_base = OTHER; note = RNA
  modified_base 16..17: mod_base = OTHER; note = DNA
  modified_base 17: mod_base = OTHER; note = inosine
  modified_base 16: mod_base = OTHER; note = 6-amino-5-nitro-3-yl-2(1H)-pyridone nucleobase
tgtccttggtccaggnnagatgcacct

SEQ ID NO: 90 - RNA, 25 nt, synthetic construct
  source 1..25: mol_type = other RNA
  modified_base order(1..15,18..25): mod_base = OTHER; note = RNA
  modified_base 16..17: mod_base = OTHER; note = DNA
  modified_base 17: mod_base = OTHER; note = inosine
  modified_base 16: mod_base = OTHER; note = 6-amino-5-nitro-3-yl-2(1H)-pyridone nucleobase
tgtccttggtccaggnnagatgcac

SEQ ID NO: 91 - RNA, 23 nt, synthetic construct
  source 1..23: mol_type = other RNA
  modified_base order(1..15,18..23): mod_base = OTHER; note = RNA
  modified_base 16..17: mod_base = OTHER; note = DNA
  modified_base 17: mod_base = OTHER; note = inosine
  modified_base 16: mod_base = OTHER; note = 6-amino-5-nitro-3-yl-2(1H)-pyridone nucleobase
tgtccttggtccaggnnagatgc

SEQ ID NO: 92 - RNA, 29 nt, synthetic construct
  source 1..29: mol_type = other RNA
  modified_base order(1..10,13..29): mod_base = OTHER; note = RNA
  modified_base 11..12: mod_base = OTHER; note = DNA
  modified_base 12: mod_base = OTHER; note = inosine
  modified_base 11: mod_base = OTHER; note = 6-amino-5-nitro-3-yl-2(1H)-pyridone nucleobase
ttggtccaggnnagatgcaccttctccgg

SEQ ID NO: 93 - RNA, 26 nt, synthetic construct
  source 1..26: mol_type = other RNA
  modified_base order(1..17,20..26): mod_base = OTHER; note = RNA
  modified_base 18..19: mod_base = OTHER; note = DNA
  modified_base 19: mod_base = OTHER; note = inosine
  modified_base 18: mod_base = OTHER; note = 6-amino-5-nitro-3-yl-2(1H)-pyridone nucleobase
cttgtccttggtccaggnnagatgca

SEQ ID NO: 94 - RNA, 27 nt, synthetic construct
  source 1..27: mol_type = other RNA
  modified_base order(1..17,20..27): mod_base = OTHER; note = RNA
  modified_base 18..19: mod_base = OTHER; note = DNA
  modified_base 19: mod_base = OTHER; note = inosine
  modified_base 18: mod_base = OTHER; note = 6-amino-5-nitro-3-yl-2(1H)-pyridone nucleobase
cttgtccttggtccaggnnagatgcac

SEQ ID NO: 95 - RNA, 28 nt, synthetic construct
  source 1..28: mol_type = other RNA
  modified_base order(1..17,20..28): mod_base = OTHER; note = RNA
  modified_base 18..19: mod_base = OTHER; note = DNA
  modified_base 19: mod_base = OTHER; note = inosine
  modified_base 18: mod_base = OTHER; note = 6-amino-5-nitro-3-yl-2(1H)-pyridone nucleobase
cttgtccttggtccaggnnagatgcacc

SEQ ID NO: 96 - RNA, 29 nt, synthetic construct
  source 1..29: mol_type = other RNA
  modified_base order(1..17,20..29): mod_base = OTHER; note = RNA
  modified_base 18..19: mod_base = OTHER; note = DNA
  modified_base 19: mod_base = OTHER; note = inosine
  modified_base 18: mod_base = OTHER; note = 6-amino-5-nitro-3-yl-2(1H)-pyridone nucleobase
cttgtccttggtccaggnnagatgcacct

SEQ ID NO: 97 - RNA, 30 nt, synthetic construct
  source 1..30: mol_type = other RNA
  modified_base order(1..17,20..30): mod_base = OTHER; note = RNA
  modified_base 18..19: mod_base = OTHER; note = DNA
  modified_base 19: mod_base = OTHER; note = inosine
  modified_base 18: mod_base = OTHER; note = 6-amino-5-nitro-3-yl-2(1H)-pyridone nucleobase
cttgtccttggtccaggnnagatgcacctt

SEQ ID NO: 98 - RNA, 31 nt, synthetic construct
  source 1..31: mol_type = other RNA
  modified_base order(1..20,22..31): mod_base = OTHER; note = RNA
  modified_base 21..22: mod_base = OTHER; note = DNA
  modified_base 22: mod_base = OTHER; note = inosine
  modified_base 21: mod_base = OTHER; note = 6-amino-5-nitro-3-yl-2(1H)-pyridone nucleobase
cgacttgtccttggtccaggnnagatgcacc

SEQ ID NO: 99 - RNA, 30 nt, synthetic construct
  source 1..30: mol_type = other RNA
  modified_base order(1..19,22..30): mod_base = OTHER; note = RNA
  modified_base 20..21: mod_base = OTHER; note = DNA
  modified_base 21: mod_base = OTHER; note = inosine
  modified_base 20: mod_base = OTHER; note = 6-amino-5-nitro-3-yl-2(1H)-pyridone nucleobase
gacttgtccttggtccaggnnagatgcacc

SEQ ID NO: 100 - RNA, 29 nt, synthetic construct
  source 1..29: mol_type = other RNA
  modified_base order(1..18,21..29): mod_base = OTHER; note = RNA
  modified_base 19..20: mod_base = OTHER; note = DNA
  modified_base 20: mod_base = OTHER; note = inosine
  modified_base 19: mod_base = OTHER; note = 6-amino-5-nitro-3-yl-2(1H)-pyridone nucleobase
acttgtccttggtccaggnnagatgcacc

SEQ ID NO: 101 - RNA, 31 nt, synthetic construct
  source 1..31: mol_type = other RNA
  modified_base order(1..19,22..31): mod_base = OTHER; note = RNA
  modified_base 20..21: mod_base = OTHER; note = DNA
  modified_base 21: mod_base = OTHER; note = inosine
  modified_base 20: mod_base = OTHER; note = 6-amino-5-nitro-3-yl-2(1H)-pyridone nucleobase
gacttgtccttggtccaggnnagatgcacct

SEQ ID NO: 102 - RNA, 30 nt, synthetic construct
  source 1..30: mol_type = other RNA
  modified_base order(1..18,21..30): mod_base = OTHER; note = RNA
  modified_base 19..20: mod_base = OTHER; note = DNA
  modified_base 20: mod_base = OTHER; note = inosine
  modified_base 19: mod_base = OTHER; note = 6-amino-5-nitro-3-yl-2(1H)-pyridone nucleobase
acttgtccttggtccaggnnagatgcacct

SEQ ID NO: 103 - RNA, 28 nt, synthetic construct
  source 1..28: mol_type = other RNA
  modified_base order(1..16,19..28): mod_base = OTHER; note = RNA
  modified_base 17..18: mod_base = OTHER; note = DNA
  modified_base 18: mod_base = OTHER; note = inosine
  modified_base 17: mod_base = OTHER; note = 6-amino-5-nitro-3-yl-2(1H)-pyridone nucleobase
ttgtccttggtccaggnnagatgcacct

SEQ ID NO: 104 - RNA, 27 nt, synthetic construct
  source 1..27: mol_type = other RNA
  modified_base order(1..16,19..27): mod_base = OTHER; note = RNA
  modified_base 17..18: mod_base = OTHER; note = DNA
  modified_base 18: mod_base = OTHER; note = inosine
  modified_base 17: mod_base = OTHER; note = 6-amino-5-nitro-3-yl-2(1H)-pyridone nucleobase
ttgtccttggtccaggnnagatgcacc

SEQ ID NO: 105 - RNA, 43 nt, Homo sapiens
  source 1..43: mol_type = transcribed RNA
ccggagaaggtgcatctcacctggaccaaggacaagtcggtgg

SEQ ID NO: 106 - DNA, 20 bp, synthetic construct
  source 1..20: mol_type = other DNA
ctcgacgcaagccataacac

SEQ ID NO: 107 - DNA, 20 bp, synthetic construct
  source 1..20: mol_type = other DNA
tggaccgactggaaacgtag

SEQ ID NO: 108 - DNA, 237 bp, synthetic construct
  source 1..237: mol_type = other DNA
tctggctcgacgcaagccataacactaatacgactcactatagggaggagcctgagggggaaggggagacagatccggagaaggtgcatctcacctggaccaaggacaagtcggtggcagagaagaataagggccccagtcctgtctcctctgagggcatcaaggacttcttcagcatgaagccggagtgggagaacttgaaccagtccaactacgtttccagtcggtccacgtttg

SEQ ID NO: 109 - DNA, 18 bp, synthetic construct
  source 1..18: mol_type = other DNA
aggagcctgagggggaag

SEQ ID NO: 110 - DNA, 21 bp, synthetic construct
  source 1..21: mol_type = other DNA
ggggcccttattcttctctgc

SEQ ID NO: 111 - DNA, 15 bp, synthetic construct
  source 1..15: mol_type = other DNA
gggaaggggagacag

SEQ ID NO: 112 - DNA, 18 bp, synthetic construct
  source 1..18: mol_type = other DNA
gtgcagctgatccacgat

SEQ ID NO: 113 - DNA, 20 bp, synthetic construct
  source 1..20: mol_type = other DNA
gcccttattcttctctgcca

SEQ ID NO: 114 - DNA, 15 bp, synthetic construct
  source 1..15: mol_type = other DNA
  modified_base 8..10: mod_base = OTHER; note = Locked Nucleic Acid
tgcatctcacctgga

SEQ ID NO: 115 - DNA, 14 bp, synthetic construct
  source 1..14: mol_type = other DNA
  modified_base 8..10: mod_base = OTHER; note = Locked Nucleic Acid
tgcatctcgcctgg

SEQ ID NO: 116 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..31): mod_base = OTHER; note = 2&apos;-O-methyl
  modified_base 21: mod_base = OTHER; note = 2&apos;-fluoro
  modified_base order(8..19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-122

SEQ ID NO: 117 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,21): mod_base = OTHER; note = 2&apos;-fluoro
  modified_base order(8..16,20,23..31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-123

SEQ ID NO: 118 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..3,17,22): mod_base = OTHER; note = 2&apos;-O-methoxyethyl
  modified_base order(4..7,21): mod_base = OTHER; note = 2&apos;-fluoro
  modified_base order(8..16,20,23..31): mod_base = OTHER; note = 2&apos;-O-methyl
  modified_base order(4..17,18^19,21..31): mod_base = OTHER; note = phosphorothioate
  modified_base 19^20: mod_base = OTHER; note = methylphosphonate
  modified_base 1: mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-124

SEQ ID NO: 119 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,16,21): mod_base = OTHER; note = 2&apos;-fluoro
  modified_base order(8..15,20,23..31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-125

SEQ ID NO: 120 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,11,13,15,21): mod_base = OTHER; note = 2&apos;-fluoro
  modified_base order(8..10,12,14,16,20,23..31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-126

SEQ ID NO: 121 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,21,26,28,30): mod_base = OTHER; note = 2&apos;-fluoro
  modified_base order(8..16,20,23..25,27,29,31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-127

SEQ ID NO: 122 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,21,26,28): mod_base = OTHER; note = 2&apos;-fluoro
  modified_base order(8..16,20,23..25,27,29..31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-128

SEQ ID NO: 123 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,11,13,15..16,21): mod_base = OTHER; note = 2&apos;-fluoro
  modified_base order(8..10,12,14,20,23..31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-129

SEQ ID NO: 124 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,16,21,26,28): mod_base = OTHER; note = 2&apos;-fluoro
  modified_base order(8..15,20,23..25,27,29..31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-130

SEQ ID NO: 125 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..10,12,14,16,20,23..31): mod_base = OTHER; note = 2&apos;-O-methyl
  modified_base order(11,13,15,21): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-131

SEQ ID NO: 126 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..10,12,14,16,20,23..25,27,29,31): mod_base = OTHER; note = 2&apos;-O-methyl
  modified_base order(11,13,15,21,26,28,30): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-132

SEQ ID NO: 127 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,27,29,31): mod_base = OTHER; note = 2&apos;-O-methyl
  modified_base order(21,26,28,30): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-133

SEQ ID NO: 128 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,27,29..31): mod_base = OTHER; note = 2&apos;-O-methyl
  modified_base order(21,26,28): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-134

SEQ ID NO: 129 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..10,12..15,20,23..25,27,29..31): mod_base = OTHER; note = 2&apos;-O-methyl
  modified_base order(11,16,21,26,28): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-135

SEQ ID NO: 130 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..12,14..15,20,23..25,27,29..31): mod_base = OTHER; note = 2&apos;-O-methyl
  modified_base order(13,16,21,26,28): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-136

SEQ ID NO: 131 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-137
cttgtccttggtccaggnnagatgcatcttc

SEQ ID NO: 132 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,6..7,17,22): mod_base = OTHER; note = 2&apos;-O-methoxyethyl
  modified_base order(5,16,21,26,28): mod_base = OTHER; note = 2&apos;-fluoro
  modified_base order(8..15,20,23..25,27,29..31): mod_base = OTHER; note = 2&apos;-O-methyl
  modified_base order(5^6,8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-138

SEQ ID NO: 133 - DNA, 31 bp, synthetic construct; identical to SEQ ID NO: 131
  source 1..31: mol_type = other DNA
  modified_base 18..19: mod_base = OTHER; note = DNA
  modified_base order(1..17,20..31): mod_base = OTHER; note = RNA
  modified_base order(1..3,17,22): mod_base = OTHER; note = 2&apos;-O-methoxyethyl
  modified_base order(4..7,21,26..29): mod_base = OTHER; note = 2&apos;-fluoro
  modified_base order(8..16,20,23..25,30..31): mod_base = OTHER; note = 2&apos;-O-methyl
  modified_base order(4..17,18^19,21..31): mod_base = OTHER; note = phosphorothioate
  modified_base 19^20: mod_base = OTHER; note = methylphosphonate
  modified_base 1: mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-139

SEQ ID NO: 134 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,11,13,15,21,26,28,30): mod_base = OTHER; note = 2&apos;-fluoro
  modified_base order(8..10,12,14,16,20,23..25,27,29,31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-140

SEQ ID NO: 135 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..4,7,17,22): mod_base = OTHER; note = 2&apos;-O-methoxyethyl
  modified_base order(5..6,11,13,21): mod_base = OTHER; note = 2&apos;-fluoro
  modified_base order(8..10,12,14..16,20,23..31): mod_base = OTHER; note = 2&apos;-O-methyl
  modified_base order(5..7,8..17,18^19,21..31): mod_base = OTHER; note = phosphorothioate
  modified_base 19^20: mod_base = OTHER; note = methylphosphonate
  modified_base order(1,7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-141

SEQ ID NO: 136 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-144

SEQ ID NO: 137 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-145

SEQ ID NO: 138 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-146

SEQ ID NO: 139 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-147

SEQ ID NO: 140 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..12,14..15,20,23..25,30..31): mod_base = OTHER; note = 2&apos;-O-methyl
  modified_base order(13,16,21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-148

SEQ ID NO: 141 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,8^9,25^26,30^31): mod_base = OTHER; note = (1,3-dimethylimidazolidin-2-ylidene) phosphoramidate linkage
  modified_base order(9..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-149

SEQ ID NO: 142 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,4^5,8^9,25^26,30^31): mod_base = OTHER; note = (1,3-dimethylimidazolidin-2-ylidene) phosphoramidate linkage
  modified_base order(9..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-150

SEQ ID NO: 143 - DNA, 31 bp, synthetic construct; identical to SEQ ID NO: 4
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,4^5,8^9,25^26,27^28,30^31): mod_base = OTHER; note = (1,3-dimethylimidazolidin-2-ylidene) phosphoramidate linkage
  modified_base order(9..17,18^19,21..25,26^27,28..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-151

SEQ ID NO: 144 - DNA, 31 bp, synthetic construct; identical to SEQ ID NO: 131
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-152

SEQ ID NO: 145 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-153
cttgtccttggtccaggnnagatgtaccttc

SEQ ID NO: 146 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-154
cttgtccttggtccaggnnagatgtatcttc

SEQ ID NO: 147 - DNA, 31 bp, synthetic construct; identical to SEQ ID NO: 4
  source 1..31: mol_type = other DNA
  modified_base 17..19: mod_base = OTHER; note = DNA
  modified_base order(1..16,20..31): mod_base = OTHER; note = RNA
  modified_base order(1..7,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-155

SEQ ID NO: 148 - DNA, 25 bp, synthetic construct
  source 1..25: mol_type = other DNA
  modified_base 7..8: mod_base = OTHER; note = DNA
  modified_base order(1..6,9..25): mod_base = OTHER; note = RNA
  modified_base order(1..4,9,12..14,19..20): mod_base = OTHER; note = 2&apos;-O-methyl
  modified_base order(5,10,15..18): mod_base = OTHER; note = 2&apos;-fluoro
  modified_base order(6,11,21..25): mod_base = OTHER; note = 2&apos;-O-methoxyethyl
  modified_base order(1^2,14^15,24^25): mod_base = OTHER; note = (1,3-dimethylimidazolidin-2-ylidene) phosphoramidate linkage
  modified_base order(2..6,7^8,10..14,15..20): mod_base = OTHER; note = phosphorothioate
  modified_base 8^9: mod_base = OTHER; note = methylphosphonate
  modified_base 7: mod_base = OTHER; note = 6-amino-5-nitro-3-yl-2(1H)-pyridone nucleobase
  modified_base 8: mod_base = OTHER; note = Inosine
  modified_base 21: mod_base = OTHER; note = m5u
  modified_base 22..23: mod_base = OTHER; note = m5c
  modified_base 1..25: mod_base = OTHER; note = B1030-156
tccaggnnagatgcaccttctccgg

SEQ ID NO: 149 - DNA, 27 bp, synthetic construct
  source 1..27: mol_type = other DNA
  modified_base 7..8: mod_base = OTHER; note = DNA
  modified_base order(1..6,9..27): mod_base = OTHER; note = RNA
  modified_base order(1..4,9,12..14,19..20): mod_base = OTHER; note = 2&apos;-O-methyl
  modified_base order(5,10,15..18): mod_base = OTHER; note = 2&apos;-fluoro
  modified_base order(6,11,21..27): mod_base = OTHER; note = 2&apos;-O-methoxyethyl
  modified_base order(1^2,14^15,26^27): mod_base = OTHER; note = (1,3-dimethylimidazolidin-2-ylidene) phosphoramidate linkage
  modified_base order(2..6,7^8,10..14,15..20): mod_base = OTHER; note = phosphorothioate
  modified_base 8^9: mod_base = OTHER; note = methylphosphonate
  modified_base 7: mod_base = OTHER; note = 6-amino-5-nitro-3-yl-2(1H)-pyridone nucleobase
  modified_base 8: mod_base = OTHER; note = Inosine
  modified_base order(21,27): mod_base = OTHER; note = m5u
  modified_base 22..23: mod_base = OTHER; note = m5c
  modified_base 1..27: mod_base = OTHER; note = B1030-157
tccaggnnagatgcaccttctccggat

SEQ ID NO: 150 - DNA, 28 bp, synthetic construct
  source 1..28: mol_type = other DNA
  modified_base 7..8: mod_base = OTHER; note = DNA
  modified_base order(1..6,9..28): mod_base = OTHER; note = RNA
  modified_base order(1..4,9,12..14,19..20): mod_base = OTHER; note = 2&apos;-O-methyl
  modified_base order(5,10,15..18): mod_base = OTHER; note = 2&apos;-fluoro
  modified_base order(6,11,21..28): mod_base = OTHER; note = 2&apos;-O-methoxyethyl
  modified_base order(1^2,14^15,27^28): mod_base = OTHER; note = (1,3-dimethylimidazolidin-2-ylidene) phosphoramidate linkage
  modified_base order(2..6,7^8,10..14,15..20): mod_base = OTHER; note = phosphorothioate
  modified_base 8^9: mod_base = OTHER; note = methylphosphonate
  modified_base 7: mod_base = OTHER; note = 6-amino-5-nitro-3-yl-2(1H)-pyridone nucleobase
  modified_base 8: mod_base = OTHER; note = Inosine
  modified_base order(21,27): mod_base = OTHER; note = m5u
  modified_base order(22..23,28): mod_base = OTHER; note = m5c
  modified_base 1..28: mod_base = OTHER; note = B1030-158
tccaggnnagatgcaccttctccggatc

SEQ ID NO: 151 - DNA, 29 bp, synthetic construct
  source 1..29: mol_type = other DNA
  modified_base 7..8: mod_base = OTHER; note = DNA
  modified_base order(1..6,9..29): mod_base = OTHER; note = RNA
  modified_base order(1..4,9,12..14,19..20): mod_base = OTHER; note = 2&apos;-O-methyl
  modified_base order(5,10,15..18): mod_base = OTHER; note = 2&apos;-fluoro
  modified_base order(6,11,21..29): mod_base = OTHER; note = 2&apos;-O-methoxyethyl
  modified_base order(1^2,14^15,28^29): mod_base = OTHER; note = (1,3-dimethylimidazolidin-2-ylidene) phosphoramidate linkage
  modified_base order(2..6,7^8,10..14,15..20): mod_base = OTHER; note = phosphorothioate
  modified_base 8^9: mod_base = OTHER; note = methylphosphonate
  modified_base 7: mod_base = OTHER; note = 6-amino-5-nitro-3-yl-2(1H)-pyridone nucleobase
  modified_base 8: mod_base = OTHER; note = Inosine
  modified_base order(21,27,29): mod_base = OTHER; note = m5u
  modified_base order(22..23,28): mod_base = OTHER; note = m5c
  modified_base 1..29: mod_base = OTHER; note = B1030-159
tccaggnnagatgcaccttctccggatct

SEQ ID NO: 152 - DNA, 30 bp, synthetic construct
  source 1..30: mol_type = other DNA
  modified_base 7..8: mod_base = OTHER; note = DNA
  modified_base order(1..6,9..30): mod_base = OTHER; note = RNA
  modified_base order(1..4,9,12..14,19..20): mod_base = OTHER; note = 2&apos;-O-methyl
  modified_base order(5,10,15..18): mod_base = OTHER; note = 2&apos;-fluoro
  modified_base order(6,11,21..30): mod_base = OTHER; note = 2&apos;-O-methoxyethyl
  modified_base order(1^2,14^15,29^30): mod_base = OTHER; note = (1,3-dimethylimidazolidin-2-ylidene) phosphoramidate linkage
  modified_base order(2..6,7^8,10..14,15..20): mod_base = OTHER; note = phosphorothioate
  modified_base 8^9: mod_base = OTHER; note = methylphosphonate
  modified_base 7: mod_base = OTHER; note = 6-amino-5-nitro-3-yl-2(1H)-pyridone nucleobase
  modified_base 8: mod_base = OTHER; note = Inosine
  modified_base order(21,27,29): mod_base = OTHER; note = m5u
  modified_base order(22..23,28): mod_base = OTHER; note = m5c
  modified_base 1..30: mod_base = OTHER; note = B1030-160
tccaggnnagatgcaccttctccggatctg

SEQ ID NO: 153 - DNA, 30 bp, synthetic construct
  source 1..30: mol_type = other DNA
  modified_base 23..25: mod_base = OTHER; note = DNA
  modified_base order(1..22,26..30): mod_base = OTHER; note = RNA
  modified_base order(1..3,6..7,10,12,16,20..21,26,29..30): mod_base = OTHER; note = 2&apos;-O-methyl
  modified_base order(4..5,8..9,11,13..15,17..19,22,27): mod_base = OTHER; note = 2&apos;-fluoro
  modified_base 28: mod_base = OTHER; note = 2&apos;-O-methoxyethyl
  modified_base order(1^2,11^12,14^15,25^26,29^30): mod_base = OTHER; note = (1,3-dimethylimidazolidin-2-ylidene) phosphoramidate linkage
  modified_base order(2..6,7..9,10^11,13^14,16..20,22..25,27..29): mod_base = OTHER; note = phosphorothioate
  modified_base 24: mod_base = OTHER; note = 6-amino-5-nitro-3-yl-2(1H)-pyridone nucleobase
  modified_base 25: mod_base = OTHER; note = Inosine
  modified_base 1..30: mod_base = OTHER; note = B1030-161
caccgacttgtccttggtccaggnnagatg

SEQ ID NO: 154 - DNA, 30 bp, synthetic construct; identical to SEQ ID NO: 153
  source 1..30: mol_type = other DNA
  modified_base 24..25: mod_base = OTHER; note = DNA
  modified_base order(1..23,26..30): mod_base = OTHER; note = RNA
  modified_base order(1..3,6..7,10,12,16,20..21,26,29..30): mod_base = OTHER; note = 2&apos;-O-methyl
  modified_base order(4..5,8..9,11,13..15,17..19,22,27): mod_base = OTHER; note = 2&apos;-fluoro
  modified_base order(23,28): mod_base = OTHER; note = 2&apos;-O-methoxyethyl
  modified_base order(1^2,11^12,14^15,29^30): mod_base = OTHER; note = (1,3-dimethylimidazolidin-2-ylidene) phosphoramidate linkage
  modified_base order(2..6,7..9,10^11,13^14,16..20,22^23,24^25,27..29): mod_base = OTHER; note = phosphorothioate
  modified_base 25^26: mod_base = OTHER; note = methylphosphonate
  modified_base 24: mod_base = OTHER; note = 6-amino-5-nitro-3-yl-2(1H)-pyridone nucleobase
  modified_base 25: mod_base = OTHER; note = Inosine
  modified_base 1..30: mod_base = OTHER; note = B1030-162

SEQ ID NO: 155 - DNA, 30 bp, synthetic construct; identical to SEQ ID NO: 153
  source 1..30: mol_type = other DNA
  modified_base 23..25: mod_base = OTHER; note = DNA
  modified_base order(1..22,26..30): mod_base = OTHER; note = RNA
  modified_base order(1..3,9,11..12,15,17,26,28..30): mod_base = OTHER; note = 2&apos;-O-methyl
  modified_base order(4..5,8,13..14,18..19,22,27): mod_base = OTHER; note = 2&apos;-fluoro
  modified_base order(6..7,10,16,20..21): mod_base = OTHER; note = 2&apos;-O-methoxyethyl
  modified_base order(1^2,11^12,14^15,25^26,29^30): mod_base = OTHER; note = (1,3-dimethylimidazolidin-2-ylidene) phosphoramidate linkage
  modified_base order(2..6,8^9,10^11,13^14,17..20,22..25,27..29): mod_base = OTHER; note = phosphorothioate
  modified_base order(7,20): mod_base = OTHER; note = m5c
  modified_base 24: mod_base = OTHER; note = 6-amino-5-nitro-3-yl-2(1H)-pyridone nucleobase
  modified_base 25: mod_base = OTHER; note = Inosine
  modified_base 1..30: mod_base = OTHER; note = B1030-163

SEQ ID NO: 156 - DNA, 30 bp, synthetic construct; identical to SEQ ID NO: 153
  source 1..30: mol_type = other DNA
  modified_base 24..25: mod_base = OTHER; note = DNA
  modified_base order(1..23,26..30): mod_base = OTHER; note = RNA
  modified_base order(1..3,9,11..12,15,17,26,28..30): mod_base = OTHER; note = 2&apos;-O-methyl
  modified_base order(4..5,8,13..14,18..19,22,27): mod_base = OTHER; note = 2&apos;-fluoro
  modified_base order(6..7,10,16,20..21,23): mod_base = OTHER; note = 2&apos;-O-methoxyethyl
  modified_base order(1^2,11^12,14^15,29^30): mod_base = OTHER; note = (1,3-dimethylimidazolidin-2-ylidene) phosphoramidate linkage
  modified_base order(2..6,8^9,10^11,13^14,17..20,22^23,24^25,27..29): mod_base = OTHER; note = phosphorothioate
  modified_base 25^26: mod_base = OTHER; note = methylphosphonate
  modified_base order(7,20): mod_base = OTHER; note = m5c
  modified_base 24: mod_base = OTHER; note = 6-amino-5-nitro-3-yl-2(1H)-pyridone nucleobase
  modified_base 25: mod_base = OTHER; note = Inosine
  modified_base 1..30: mod_base = OTHER; note = B1030-164

SEQ ID NO: 157 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base 2..5: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-165
cttttccttggtccaggnnagatgcaccttc

SEQ ID NO: 158 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,4,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-166
cttctccttggtccaggnnagatgcaccttc

SEQ ID NO: 159 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-167
cttgtccctggtccaggnnagatgcaccttc

SEQ ID NO: 160 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base 2..3: mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-168
cttgaccttggtccaggnnagatgcaccttc

SEQ ID NO: 161 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17): mod_base = OTHER; note = 2&apos;-O-methoxyethyl
  modified_base order(8..16,20,22..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-169
cttgtccttggtccaggnnagctgcaccttc

SEQ ID NO: 162 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-170
cttatccctggtccaggnnagatgcaccttc

SEQ ID NO: 163 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17): mod_base = OTHER; note = 2&apos;-O-methoxyethyl
  modified_base order(8..16,20,22..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-171
cttatccctggtccaggnnagctgcaccttc

SEQ ID NO: 164 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = B1030-172
cttatccctggtccaggnnagatgtatcttc

SEQ ID NO: 165 - DNA, 31 bp, synthetic construct
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..31): mod_base = OTHER; note = 2&apos;-O-methyl
  modified_base 21: mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-4
cttatccttggtccaggnnagatgcaccttc

SEQ ID NO: 166 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..10,20,29..31): mod_base = OTHER; note = 2&apos;-O-methyl
  modified_base order(11..16,21,23..28): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-26

SEQ ID NO: 167 - DNA, 32 bp, synthetic construct
  source 1..32: mol_type = other DNA
  modified_base 19..20: mod_base = OTHER; note = DNA
  modified_base order(1..18,21..32): mod_base = OTHER; note = RNA
  modified_base order(1..8,18,23): mod_base = OTHER; note = 2&apos;-O-methoxyethyl
  modified_base order(9..11,21,30..32): mod_base = OTHER; note = 2&apos;-O-methyl
  modified_base order(12..17,22,24..29): mod_base = OTHER; note = 2&apos;-fluoro
  modified_base order(1^2,26^27,31^32): mod_base = OTHER; note = (1,3-dimethylimidazolidin-2-ylidene) phosphoramidate linkage
  modified_base order(9..18,19^20,22..26,27..31): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..32: mod_base = OTHER; note = rB1030-39
acttatccttggtccaggnnagatgcaccttc

SEQ ID NO: 168 - DNA, 30 bp, synthetic construct
  source 1..30: mol_type = other DNA
  modified_base 19..20: mod_base = OTHER; note = DNA
  modified_base order(1..18,21..30): mod_base = OTHER; note = RNA
  modified_base order(1..8,18,23): mod_base = OTHER; note = 2&apos;-O-methoxyethyl
  modified_base order(9..11,21,30): mod_base = OTHER; note = 2&apos;-O-methyl
  modified_base order(12..17,22,24..29): mod_base = OTHER; note = 2&apos;-fluoro
  modified_base order(9..18,19^20,22..30): mod_base = OTHER; note = phosphorothioate
  modified_base 20^21: mod_base = OTHER; note = methylphosphonate
  modified_base order(2,7..8): mod_base = OTHER; note = m5c
  modified_base order(3..4,6): mod_base = OTHER; note = m5u
  modified_base 19: mod_base = OTHER; note = 6-amino-5-nitro-3-yl-2(1H)-pyridone nucleobase
  modified_base 20: mod_base = OTHER; note = Inosine
  modified_base 1..30: mod_base = OTHER; note = rB1030-50
acttatccttggtccaggnnagatgcacct

SEQ ID NO: 169 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(1..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-65

SEQ ID NO: 170 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-66

SEQ ID NO: 171 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,29..31): mod_base = OTHER; note = 2&apos;-O-methyl
  modified_base order(21,23..28): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-67

SEQ ID NO: 172 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-68

SEQ ID NO: 173 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,29..31): mod_base = OTHER; note = 2&apos;-O-methyl
  modified_base order(21,23..28): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-69

SEQ ID NO: 174 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,29..31): mod_base = OTHER; note = 2&apos;-O-methyl
  modified_base order(21,23..28): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-70

SEQ ID NO: 175 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-72

SEQ ID NO: 176 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(8..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-73

SEQ ID NO: 177 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-74

SEQ ID NO: 178 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..11,13,15,20,29..31): mod_base = OTHER; note = 2&apos;-O-methyl
  modified_base order(12,14,16,21,23..28): mod_base = OTHER; note = 2&apos;-fluoro
  modified_base order(1^2,30^31): mod_base = OTHER; note = (1,3-dimethylimidazolidin-2-ylidene) phosphoramidate linkage
  modified_base order(2..17,18^19,21..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-77

SEQ ID NO: 179 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..11,13,15,20,29..31): mod_base = OTHER; note = 2&apos;-O-methyl
  modified_base order(12,14,16,21,23..28): mod_base = OTHER; note = 2&apos;-fluoro
  modified_base order(17,22): mod_base = OTHER; note = 2&apos;-O-methoxyethyl
  modified_base order(1^2,30^31): mod_base = OTHER; note = (1,3-dimethylimidazolidin-2-ylidene) phosphoramidate linkage
  modified_base order(2..17,18^19,21..30): mod_base = OTHER; note = phosphorothioate
  modified_base 19^20: mod_base = OTHER; note = methylphosphonate
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-78

SEQ ID NO: 180 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..16,20,23..25,30..31): mod_base = OTHER; note = 2&apos;-O-methyl
  modified_base order(21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-144

SEQ ID NO: 181 - DNA, 31 bp, synthetic construct; identical to SEQ ID NO: 165
  source 1..31: mol_type = other DNA
  modified_base 18..19: mod_base = OTHER; note = DNA
  modified_base order(1..17,20..31): mod_base = OTHER; note = RNA
  modified_base order(1..7,17,22): mod_base = OTHER; note = 2&apos;-O-methoxyethyl
  modified_base order(8..15,20,23..25,30..31): mod_base = OTHER; note = 2&apos;-O-methyl
  modified_base order(16,21,26..29): mod_base = OTHER; note = 2&apos;-fluoro
  modified_base order(1^2,25^26,30^31): mod_base = OTHER; note = (1,3-dimethylimidazolidin-2-ylidene) phosphoramidate linkage
  modified_base order(8..17,18^19,21..25,26..30): mod_base = OTHER; note = phosphorothioate
  modified_base 19^20: mod_base = OTHER; note = methylphosphonate
  modified_base order(1,6..7): mod_base = OTHER; note = m5c
  modified_base order(2..3,5): mod_base = OTHER; note = m5u
  modified_base 18: mod_base = OTHER; note = 6-amino-5-nitro-3-yl-2(1H)-pyridone nucleobase
  modified_base 19: mod_base = OTHER; note = Inosine
  modified_base 1..31: mod_base = OTHER; note = rB1030-145

SEQ ID NO: 182 - DNA, 18 bp, synthetic construct
  source 1..18: mol_type = other DNA
gtgcagctgatccatgac

SEQ ID NO: 183 - DNA, 20 bp, synthetic construct
  source 1..20: mol_type = other DNA
gcctttgttcttctgagccg

SEQ ID NO: 184 - RNA, 31 nt, synthetic construct
  source 1..31: mol_type = other RNA
  modified_base order(1..24,27..31): mod_base = OTHER; note = RNA
  modified_base 25..26: mod_base = OTHER; note = DNA
  modified_base 26: mod_base = OTHER; note = inosine
  modified_base 25: mod_base = OTHER; note = 6-amino-5-nitro-3-yl-2(1H)-pyridone nucleobase
ccaccgacttgtccttggtccaggnnagatg

(19) United States
(12) Patent Application Publication
(10) Pub. No.: US 2026/0275339 A1
(43) Pub. Date: Sep. 17, 2026

(54) ANTISENSE OLIGONUCLEOTIDES FOR THE TREATMENT OF NEUROLOGICAL DISORDERS

(71) Applicants: ProQR Therapeutics II B.V., Leiden (NL); Eli Lilly and Company, Indianapolis, IN (US)

(72) Inventors: Aron KOS, Leiden (NL); Lenka VAN SINT FIET, Leiden (NL); Lisanne Alieda VAN WISSEN, Leiden (NL); Marieke HOGERVORST, Leiden (NL); Ryan Matthew SMITH, Indianapolis, IN (US)

(73) Assignees: ProQR Therapeutics II B.V., Leiden (NL); Eli Lilly and Company, Indianapolis, IN (US)

(21) Appl. No.: 19/168,359

(22) PCT Filed: Mar. 22, 2024
(86) PCT No.: PCT/US2024/021210

§ 371 Date: Sep. 24, 2025

Related U.S. Application Data
(60) Provisional application No. 63/492,019, filed on Mar. 24, 2023.

(51) Int. Cl. C12N 15/113

(52) U.S. Cl. CPC C12N 15/113; C12N 2310/11; C12N 2310/3125; C12N 2310/313; C12N 2310/314; C12N 2310/315; C12N 2310/321; C12N 2310/322

(57) ABSTRACT

The disclosure relates to the field of diseases caused by a lowered synaptic inhibition, preferably those that are caused by a diminished activity of the potassium (K)/chloride (Cl) cotransporter (KCC2). The disclosure involves oligonucleotides and the use thereof in RNA editing methods in targeting a target adenosine in a codon encoding a phosphorylation site in the KCC2-encoding SLC12A5 pre-mRNA or mRNA, preferably the adenosine in the codon encoding threonine at position 1007 of the KCC2b isoform. Through the editing the threonine is replaced by an alanine, thereby removing the phosphorylation site, and thereby increasing the activity of the KCC2 protein in the process of restoring GABAergic inhibitory tone. The disclosure further relates to oligonucleotides for use in the treatment of chronic pain and epilepsy.

### CROSS-REFERENCE TO RELATED APPLICATIONS AND INCORPORATION BY REFERENCE

This PCT application claims the priority benefit of U.S. Provisional Application No. 63/492,019, filed on Mar. 24, 2023, which is herein incorporated by reference in its entirety.

### REFERENCE TO SEQUENCE LISTING SUBMITTED ELECTRONICALLY

The content of the electronically submitted ST.26 sequence listing in xml format (name 0058WO01ORD_20240315xmL.xml; size: 893,403 bytes; and date of creation: Mar. 18, 2023) filed with the application is incorporated herein by reference in its entirety.

### TECHNICAL FIELD

This disclosure relates to the field of medicine, and in particular to the field of neurological disorders. The disclosure describes antisense oligonucleotides that mediate nucleotide-specific RNA editing in the human SLC12A5 gene transcript to bring about amino acid changes of the encoded KCC2 protein that influence its activity.

### BACKGROUND

Fast synaptic transmission relies on ion fluxes through ligand-gated channels. Therefore, maintaining transmembrane ionic gradients is critical to preserve synaptic efficacy. In the adult vertebrate Central Nervous System (CNS), the potassium (K)/chloride (Cl) cotransporter (KCC2) is highly enriched in neurons and continuously extrudes Cl ions (Cl⁻), thus ensuring that intracellular levels of Cl⁻ remain low, as required for inhibitory γ-aminobutyric acid (GABA)-ergic and glycinergic neurotransmission. The Cl-gradient determines whether GABA receptor and glycine receptor (GlyR) generate inhibitory or excitatory signals within the brain. In the developed CNS, maintaining the inhibitory tone of GABA receptors and GlyR is critical for normal neuronal function. Disruption of inhibitory signalling is associated with a wide range of neurological and psychiatric disorders. A key mechanism that has emerged in the understanding of the underlying mechanism of disinhibition involves the disruption of Cl⁻ homeostasis resulting from the loss of activity of KCC2. This mechanism appears across several pathological pain syndromes with diverse etiologies, including spinal cord injury (Coull J A et al. 2003. Nature 424 (6951): 938-942), inflammation (Lu Y et al. 2008. J Physiol (Lond.) 586 (Pt 23): 5701-5715), painful diabetic neuropathy (Jolivalt C G et al. 2008. Pain 140 (1): 48-57), trigeminal pain (Wei B et al. 2013. Neuroscience 228:334-348), morphine-induced hyperalgesia (Ferrini F et al. 2013. Nat Neurosci. 16 (2): 183-192), and epilepsy (Cohen I et al. 2002. Science 298:1418-1421; Huberfeld G et al. 2007. J Neurosci. 27 (37): 9866-9873). Since KCC2 is responsible for Cl-extrusion, disruption of its function causes a collapse of the transmembrane Cl⁻ gradient and a depolarizing shift in GABAA reversal potential (EGABA). This in turn leads to a decrease in inhibitory efficacy. In chronic pain, GABA-ergic transmission is compromised, causing circuit malfunction, and disrupting inhibitory neural networks. Additionally, it is found that in chronic pathologic pain KCC2 expression is attenuated in the primary sensory gate in spinal cord dorsal horn (SCDH) neurons. It is therefore well established that this key pathophysiological mechanism contributes to an imbalance of excitation/inhibition because it corrupts inhibitory neurotransmission, leading to inhibitory circuit malfunction. Notably, there is no ‘back-up’ protein that can rescue the KCC2 expression deficit.

There are two isoforms of KCC2: KCC2a and KCC2b that arise from alternative transcriptional start sites within the human SLC12A5 gene. These transcripts translate to two protein isoforms that differ in their N-termini, with the KCC2a form constituting the larger of the two splice variants. KCC2a levels remain relatively constant during pre- and postnatal development, whereas KCC2b, on the other hand, is scarcely present during prenatal development and is strongly upregulated during postnatal development. The upregulation of KCC2b expression is thought to be responsible for the ‘developmental shift’ observed in mammals from depolarizing postsynaptic effects of inhibitory synapses in early neural networks to hyperpolarizing effects in mature neural networks. It has been established that besides its function in regulating intraneuronal Cl⁻ homeostasis, the activity of KCC2 is also associated with transmembrane water fluxes that compensate solute fluxes associated with synaptic activity. Moreover, KCC2 interaction with the actin cytoskeleton appears critical both for dendritic spine morphogenesis and the maintenance of glutamatergic synapses (Chamma I et al. 2012. Front Cell Neurosci. 6:5). KCC2b knockout mice can survive up to postnatal day 17 due to the presence of functional KCC2a alone, but they exhibit low body weight, motor deficits and generalized seizures. Complete KCC2 knockouts, in which both KCC2a and KCC2b are absent, die after birth due to respiratory failure.

Enhancing KCC2 activity can potentially be used as treatment of a wide variety neurological disorders where a lower inhibitory tone exacerbates or is the underlying cause of the disease. For example, increasing KCC2 function has been proposed for treatment of pathogenic pain (Doyon N et al. 2013. Expert Rev Neurother. 13 (5): 469-471). Here, enhancing inhibitory signalling through increasing KCC2 function alleviates deficits in GABAA and glycine inhibitory signalling observed in neuropathic pain (Lorenzo L-E et al. 2020. Nature Communications 11:869). In epilepsy where seizures are induced by increased excitatory neuronal activity or by a reduction in inhibitory tone, increasing KCC2 has been proposed as therapeutic strategy (Moore Y E et al. 2017. Trends Neurosci. 40 (9): 555-571). For example, genetic removal of KCC2 activity impairing phosphorylation sites, was shown to be sufficient to limit the onset and severity of seizures (Moore Y E et al. 2018. Proc Natl Acad Sci USA. 115 (40): 10166-10171). Moreover, in the neurodevelopmental disorders such as autism spectrum disorder (ASD) and Rett syndrome (a severe form of ASD), impaired GABAergic inhibitory function has been observed (Tyzio R et al. 2014. Science 343 (6171): 675-679; Tang X et al. 2016. Proc Natl Acad Sci USA. 113 (3): 751-756). In post-mortem brain samples from Rett syndrome patients, a significantly lower expression of KCC2 was observed (Hinz L et al. 2019. Acta Neuropathol Commun. 7 (1): 196) and restoring KCC2 function in neurons derived from patients with Rett syndrome was shown to restore functional deficits observed in these neurons (Tang et al. 2016).

Despite the use of the small molecule, and KCC2 ‘activator’ CLP257 that appeared not to act on KCC2 itself, and despite the numerous attempts to increase KCC2 activity in individuals suffering from neurological diseases such as chronic pain and epilepsy, there remains a need for alternatives to treat these severe quality-of-life related disorders. The present disclosure aims to provide such alternative, and/or improved, compounds and compositions for use in the treatment of neuronal disorders in which an increase in KCC2 activity is beneficial.

### BRIEF SUMMARY

Disclosed herein is an RNA editing oligonucleotide (EON) capable of forming a double-stranded complex with a region of an endogenous human SLC12A5 transcript molecule in a cell, wherein the region of the SLC12A5 transcript molecule comprises a target adenosine, and wherein the double-stranded complex can recruit an endogenous ADAR enzyme to deaminate the target adenosine (A) into an inosine (I), thereby editing the SLC12A5 transcript molecule. Preferably, the SLC12A5 transcript molecule is a pre-mRNA or an mRNA molecule, and preferably, the SLC12A5 transcript molecule has a wildtype sequence. In a preferred aspect, the target A is in a codon encoding an amino acid that can be phosphorylated, more preferably wherein the target A is a first nucleotide of the codon encoding threonine at position 1007 of the SLC12A5-encoded KCC2b isoform according to the sequence referenced by NCBI Ref. Seq. No. NP_065759.1. In another aspect, the target A is a first nucleotide of the codon encoding threonine at position 1030 of the SLC12A5-encoded KCC2a isoform as referenced by NCBI Ref. Seq. No. NP_001128243.1.

Disclosed herein is also an EON according to the disclosure for use in the treatment of a disorder caused by a diminished GABAergic inhibition, preferably caused by a diminished KCC2 activity, preferably wherein the disorder is chronic pain or epilepsy.

Disclosed herein is also a method of editing a SLC12A5 polynucleotide, the method comprising contacting the SLC12A5 polynucleotide with an EON capable of effecting an ADAR-mediated A to I editing of a target A in a codon encoding an amino acid that is associated with phosphorylation of the SLC12A5-encoded protein KCC2, thereby editing the SLC12A5 polynucleotide. Disclosed herein is also a method of treating a disorder caused by a diminished GABAergic inhibition, preferably caused by a diminished KCC2 activity, in an individual in need thereof, the method comprising contacting a SLC12A5 polynucleotide in a cell of the subject with an EON capable of effecting an ADAR-mediated A to I editing of a target A in a codon encoding an amino acid that is associated with phosphorylation of the SLC12A5-encoded protein KCC2, thereby treating the individual. Disclosed is also a method of deaminating a target A in an SLC12A5 pre-mRNA or mRNA molecule in a cell, the method comprising the steps of: (i) providing the cell with an EON as disclosed herein, (ii) allowing uptake by the cell of the EON, (iii) allowing annealing of the EON to the SLC12A5 pre-mRNA or mRNA molecule, (iv) allowing an endogenous ADAR enzyme to deaminate the target A in the target RNA molecule to an I; and optionally (v) identifying the presence of the I in the target RNA molecule.

### BRIEF DESCRIPTION OF THE DRAWINGS

One or more embodiments will now be described, by way of example only, with reference to the accompanying drawings, in which:

FIGS. 1A and 1B show on top part of the human SLC12A5 (pre-) mRNA target transcript sequence (5′ to 3′; SEQ ID NO: 105) including the target A in bold face and the threonine encoding codon underlined. Below the target sequence, in FIG. 1A the sequences (also 5′ to 3′) are given of an initial 78 editing oligonucleotides (EONs B1 to B78) that were designed to bring about editing of the target A. The SEQ ID NO of each of the modified EONs is given between brackets. The chemical modifications in the EONs are as follows: m5Ce is 2′-MOE modified 5-methylcytidine; m5Ue is 2′-MOE modified 5-methyluridine (Te; 2′-MOE modified thymidine); Ge is 2′-MOE modified guanosine; Ae is 2′-MOE modified adenosine; Gm, Am, Um, and Cm are 2′-OMe modified guanosine, adenosine, uridine, and cytidine, respectively; Af, Uf, Gf, Cf, and If are 2′-F modified adenosine, uridine, guanosine, cytosine, and inosine, respectively; Zd is a cytidine analog that is also referred to as a nucleoside carrying a Benner's base (as further outlined herein), with a deoxy moiety (=DNA) at the 2′ ribose position; Id is deoxyinosine; * refers to a phosphorothioate (PS) linkage; ! refers to a (1,3-dimethylimidazolidin-2-ylidene) phosphoramidate (PNdmi) linkage; \hat{ } refers to a methylphosphonate (MP) linkage. All other linkages are phosphodiester (PO) linkages. FIG. 1B shows the nucleotide sequences (SEQ ID NO: 79 to 104) of the editing oligonucleotides from FIG. 1A without any chemical modifications, except for the Zd (Z) and Id (I) positions. SEQ ID NO:82 is also the sequence of the EONs of SEQ ID NO:141 to 147 without any chemical modifications, except for the Zd (Z) and Id (I) positions. Shown is also SEQ ID NO: 184, which is the sequence of the EONs of SEQ ID NO: 154, 155, and 156 without any chemical modifications, except for the Zd (Z) and Id (I) positions.

FIGS. 2A to 2E show percentage editing, over time, in an in vitro biochemical editing assay using EONs B1 to B20 shown in FIG. 1A, and in vitro generated SLC12A5 transcript RNA, applying added purified ADAR enzyme. FIG. 2A shows percentage editing for EONs B1, B2, B3, B4, and B5. FIG. 2B shows percentage editing for EONs B6, B7, B8, B9, and B10. FIG. 2C shows percentage editing for EONs B6, B11, B12, and B13. FIG. 2D shows percentage editing for EONs B6, B14, B15, B16, and B17. FIG. 2E shows percentage editing for EONs B18, B19, and B20.

FIGS. 3A and 3B show the percentage editing measured after 14 days upon EON treatment in 200-days cultured human retinal organoids, in a first screen, using gymnotic uptake of the EONs from the culture medium. Tested EONs are mentioned in each figure. FIG. 3A shows percentage editing for EONs B1, B2, B3, B4, B5, B6, B7, B8, B9, B10, B11, B12, and B13 shown in FIG. 1A. FIG. 3B shows percentage editing for EONs B6, B15, B16, B17, B18, B19 and B20 shown in FIG. 1A.

FIG. 4 shows the percentage editing measured after 14 days upon EON treatment in 200-days cultured human retinal organoids, in a second screen, using gymnotic uptake of the EONs from the culture medium. Tested EONs are B3, B14, B21, B22, B23, B24, B25, B26, B27, B28, B29, B30, B31, B32, B33, B34, B35, B36, B37, B38, B39 and B40 shown in FIG. 1A, in which B3 was also used in the screen shown in FIG. 3A.

FIGS. 5A and 5B show the editing percentages in HEK cells that stably over-express human KCC2 using the 78 EONs depicted in FIG. 1A, 24 hrs after transfection of the EONs into the cells. FIG. 5A shows the results with B1 to B40, and FIG. 5B shows the results with B41 to B78, both with the mock transfection as the negative control.

FIG. 6A shows the editing percentage in HEK-KCC2 cells that were transfected with the indicated EONs, 48 hrs after transfection. The same transfected cell samples were used to determine the effect on the amount of phosphorylated KCC2 upon transfection with the specified EONs. FIG. 6B shows the normalized phosphorylated KCC2 (referred to here as Target B) divided by total KCC2 in comparison to the mock transfected cells, set here as 100. The normalized values are given within each bar.

FIGS. 7A and 7B show the editing percentages in human iPSC neurons using the 78 EONs depicted in FIG. 1A, after two weeks of gymnotic exposure of the indicated EONs using a washout procedure. FIG. 7A shows the results with B1 to B40 and FIG. 7B shows the results with B41 to B78, both with the non-treated (NT) sample as the negative control. EON B51 was not available at the time of the experiment and editing percentages are not provided for this EON.

FIG. 8 shows a set of EONs (B122 to B137, B140, and B141, with their respective SEQ ID NO's given between brackets) based on EON B4 (see FIG. 1A) that is shown on top. The EONs have a variety of 2′-F modifications throughout the designs. The 2′-F modified nucleotides are given with grey boxes. The chemical modifications are as provided in FIG. 1A.

FIG. 9 shows the editing percentages obtained in human iPSC neurons that were gymnotically treated with the EONs provided in FIG. 8, with a washout treatment of 2 weeks. A non-treated (NT) sample was taken along as the negative control.

FIG. 10 shows a set of EONs (B1030-144 to B1030-172; also referred to as B144 to B172, respectively; with their respective SEQ ID NO's between brackets) roughly based on the design of B137 (see FIG. 8). The EONs have a variety of 2′-F modifications, mismatches/wobbles, PNdmi linkages, and 2′-deoxy modifications at different positions as indicated. The chemical modifications are as provided in FIG. 1A.

FIG. 11 shows the editing percentages obtained in human iPSC neurons that were gymnotically treated with the EONs provided in FIG. 10, as indicated, with a washout experiment of 2 weeks. A non-treated sample and an EON that was specific for the equivalent rat Slc12a5 target sequence (rB1030-144) were taken along. B1030-152 was initially not manufactured and therefore not tested.

FIG. 12 shows the sequence of a set of EONs with their respective SEQ ID NO between brackets that were designed to target the equivalent A, in vivo, in the rat Slc12a15 transcript (in comparison to the human transcript) resembling the change of the codon for threonine at position 1007 to a codon for alanine. The names of the EONs resemble the same names as their equivalent EONs used to target the human transcript molecule. For example, rB1030-4 has the same chemical modifications as B4 in FIG. 1A but comprises a 2′-MOE modified adenosine (Ae; underlined) at position +14 instead of a 2′-MOE modified guanosine (Ge).

FIG. 13 shows the editing percentages in the lumbar spinal cord in rats two weeks after intrathecal administration (directly in the spinal cord) of a single dose of 300 μg EON, as indicated. HD indicates a higher administered dose, as discussed in the examples. All EONs provided in FIG. 12 were tested together with three EONs that are complementary to the human SLC12A15 target sequence (B-70, B-74, and B-145). Many injections were off-site and accidentally besides the spinal cord, which gives 0 editing. These mis-injections were not taken along in the editing calculations. Artificial cerebrospinal fluid (aCSF), which was also the buffer in which the EONs were dissolved, served as a negative control.

### DETAILED DESCRIPTION

The KCC2 protein is extensively post-transcriptionally modified, and the functional properties of KCC2 is reciprocally regulated by serine/threonine phosphorylation. One site that is post-translationally phosphorylated is the threonine residue at position 1007 in the human KCC2b isoform (see, NCBI Ref. Seq. No. NP_065759.1) that is equivalent to the threonine at position 1030 in the human KCC2a isoform (see, NCBI Ref. Seq. No. NP_001128243.1). Hereinafter, because the targeting discussed is predominantly for transcripts present in mature neuronal cells, the target threonine is generally referred to as being at position 1007 (in KCC2b), but it is to be understood that the equivalent threonine at position 1030 in KCC2a may also be changed with the compounds and compositions as disclosed herein, and that when the disclosure refers to targeting the threonine (or the adenosine in the codon coding for the threonine) that both isoforms are included. It has been demonstrated that phosphorylation of this site leads to decreased activity of the KCC2 channel resulting in decreased inhibitory tone (Pisella L I et al. 2019. Sci Signal. 12(603):eaay0300). Inversely, it was shown that disruption of this phosphorylation site enhances KCC2 function and in turn increases inhibitory signalling (Weber M et al. 2014. J Biol Chem 289(27):18668-18679; Moore et al. 2018., supra), which together lead to the realization by the inventors that post-transcriptionally changing the threonine at this position to a different residue (that could not be phosphorylated) would in principle make that the KCC2 protein more active in its inhibitory effect and would therethrough allow a potential treatment of disorders in which KCC2 activity is diminished, either through lowered expression, loss-of-function mutations or through post-translational modification processes, such as an increased phosphorylation rate of the threonine at position 1007. Because of the plethora of disorders caused by a lowered KCC2 activity, as discussed above, any of such disorders could potentially be treated when the KCC2 activity could (transiently) be upregulated to yield a higher inhibitory effect, even when the human SLC12A5 gene, encoding KCC2, is wild type.

The technology that the inventors envisioned is generally referred to as ‘RNA editing’, in which a specific adenosine present in a transcript molecule, such as a pre-mRNA or a mRNA molecule, is deaminated to an inosine, which is seen by the translation machinery as a guanosine. Hence, by editing the ACC codon for the threonine residue at position 1007 in KCC2b (or position 1030 in KCC2a) to an ICC codon (=GCC) in the transcript, the resulting protein would comprise an alanine residue at this position instead of a threonine and the protein can no longer be phosphorylated at this site. It is noted that the threonine to alanine change is not a mutation that has thus far been identified in nature, but the inventors realized that the change should reflect a gain-of-function alteration of the protein, which technically cannot be induced in human subjects through knock-in procedures as outlined in Moore et al. (2018), supra, or by gene therapy. The RNA editing technology provides a unique transient method of altering the KCC2 protein in the CNS of human individuals in need thereof, preferably in the treatment of (chronic) pain and/or seizure (epilepsy) disorders, without altering the individual's genome. It also allows the treatment of CNS disorders in which the KCC2 protein is attenuated in its expression or mutated towards a loss-of-function mutant, such as those identified by Stodberg and colleagues (2015. Nat Commun 6:8038) and Saitsu and colleagues (2016. Sci Rep 6:30072).

In one embodiment, the disclosure relates to EONs that are used to specifically cause the deamination of a specific target adenosine in the transcript of the (human) mutant SLC12A5 transcript (pre-mRNA and/or mRNA) in vivo, using endogenous deaminating enzymes (see below), to produce a KCC2 protein that will not be phosphorylated at the position encoded by the codon in which the adenosine was present. The resulting KCC2 protein (be it the KCC2a and/or the KCC2b isoform) is then enhanced in its inhibitory signalling function.

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A)-to-inosine (I) conversions and cytidine (C)-to-uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded (ds) RNA recognition domains, depending on the enzyme in question. Each recognition domain recognizes a specific dsRNA sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. As mentioned above, inosine is read as guanosine by the translational machinery of the cell, meaning that, if an edited adenosine is in a coding region of an mRNA or pre-mRNA, it can recode the protein sequence. A-to-I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A-to-I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of antisense oligonucleotides to edit a target RNA applying adenosine deaminase has been described (e.g., Woolf et al. 1995. PNAS 92:8298-8302; Montiel-Gonzalez et al. PNAS 2013, 110 (45): 18285-18290; Vogel et al. 2014. Angewandte Chemie Int Ed 53:267-271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences suffered from severe lack of specificity: nearly all adenosines in the target RNA strand complementary to the oligonucleotide were edited.

It is known that ADAR may act on any dsRNA. Through a process sometimes referred to as ‘promiscuous editing’, the enzyme will edit multiple adenosines in the dsRNA. Hence, there was a need for methods and means that circumvent such promiscuous editing and only target specific adenosines in a target RNA molecule to become therapeutic applicable. Vogel et al. (2014) showed that such off-target editing can be suppressed by using 2′-O-methyl (2′-OMe) modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the oligonucleotides.

Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. Intl. Patent Application Publication No. WO2016/097212, which is herein incorporated by reference in its entirety, discloses oligonucleotides for the targeted editing of RNA, wherein the oligonucleotides are characterized by a sequence that is complementary to a target RNA sequence (therein referred to as the ‘targeting portion’) and by the presence of a stem-loop (or hairpin) structure (therein referred to as the ‘recruitment portion’), which is preferably non-complementary to the target RNA. Such oligonucleotides are referred to as “self-looping oligonucleotides”. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. Intl. Patent Application Publication No. WO2016/097212, which is herein incorporated by reference in its entirety, describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the oligonucleotide itself, and are thought to attract (endogenous) ADAR. Similar stem-loop structure-comprising systems for RNA editing have been described in Intl. Patent Application Nos. WO2017/050306, WO2020/001793, WO2017/010556, WO2020/246560, and WO2022/078995, all of which are herein incorporated by reference in their entireties.

Intl. Patent Application Nos. WO2017/220751 and WO2018/041973, which are herein incorporated by reference in their entireties, describe a next generation type of oligonucleotides that do not comprise such a stem-loop structure but that are (almost fully) complementary to the targeted area. In one embodiment, one or more mismatching nucleotides, wobbles, or bulges exist between the oligonucleotide and the target sequence. A sole mismatch may be at the site of the nucleoside opposite the target adenosine, but in other embodiments oligonucleotides (often referred to as “RNA editing oligonucleotides”, abbreviated to ‘EONs’, although they do not have the enzymatic deamination or editing activity themselves) were described with multiple bulges and/or wobbles when attached to the target sequence area. It appeared possible to achieve in vitro, ex vivo and in vivo RNA editing with EONs lacking a stem-loop structure and with endogenous ADAR enzymes when the sequence of the EON was carefully selected such that it could attract/recruit ADAR. The “orphan nucleoside”, which is defined as the nucleoside in the EON that is positioned directly opposite the target adenosine in the target RNA molecule, did not carry a 2′-OMe modification. The orphan nucleoside can be a deoxyribonucleoside (DNA) without any substitution at the 2′ position of the ribose sugar moiety, wherein the remainder of the EON could still carry 2′-O-alkyl modifications (such as 2′-OMe) at their ribose sugars. The nucleotides directly surrounding the orphan nucleoside contained chemical modifications (including being DNA and not RNA) that further improved the RNA editing efficiency and/or increased the resistance against nucleases. Such effects could even be further improved by using sense oligonucleotides (SONs) that protected the EONs against breakdown (described in WO2018/134301). The use of chemical modifications and particular structures in oligonucleotides that could be used in ADAR-mediated editing of specific adenosines in a target RNA have been the subject of numerous publications in the field, such as Intl. Patent Application Nos. WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345, all of which are herein incorporated by reference in their entireties. The use of specific sugar moieties has been disclosed in for instance Intl. Patent Application Nos. WO2020/154342, WO2020/154343, WO2020/154344, WO2022/103839, and WO2022/103852, which are herein incorporated by reference in their entireties, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in Intl. Patent Application Nos. WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159, which are herein incorporated by reference in their entireties. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are Intl. Patent Application Nos. WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1), which are herein incorporated by reference in their entireties.

Disclosed herein is an RNA editing oligonucleotide (EON) capable of forming a double-stranded complex with a region of an endogenous human SLC12A5 transcript molecule in a cell, wherein the region of the SLC12A5 transcript molecule comprises a target adenosine, wherein the nucleotide in the EON that is directly opposite the target adenosine is the orphan nucleotide, wherein the counting of the nucleotides in the EON is such that the orphan nucleotide is number 0 and the nucleotides 5′ from the orphan nucleotide are positively (+) incremented towards the 5′ end and negatively (−) incremented towards the 3′ end, and wherein the double-stranded complex can recruit an endogenous ADAR enzyme to deaminate the target adenosine into an inosine, thereby editing the SLC12A5 transcript molecule. Preferably, the SLC12A5 transcript molecule is a pre-mRNA or an mRNA molecule. In one aspect, the SLC12A5 transcript molecule has a wildtype sequence. In one aspect, the target adenosine is in a codon encoding an amino acid that can be phosphorylated. Preferably, the target adenosine is a first nucleotide of the codon encoding threonine at position 1007 of the SLC12A5-encoded KCC2b isoform. In an alternative aspect, the target adenosine is a first nucleotide of the codon encoding threonine at position 1030 of the SLC12A5-encoded KCC2a isoform. In one aspect, the deamination of the target adenosine results in an SLC12A5-encoded KCC2 protein with an increased activity. Preferably, the increased activity results in a higher GABAergic inhibition. In one aspect, the cell in which the editing of the SLC12A5 transcript editing occurs is a (human) neuron, preferably a (human) brain cell. In one aspect, the EON is selected from the group consisting of SEQ ID NO: 1 to 104, and 116 to 164. In a preferred aspect, the EON is selected from the group consisting of SEQ ID NO:3, 4, 14, 15, 22, 23, 28, 29, 33, 34, 35, 36, 40, 55, 63, 65, 69, 70, 73, 74, 77, 78, 79, 80, 81, 82, 83, 84, 85, 86, 87, 88, 89, 90, 91, 92, 93, 94, 95, 96, 97, 98, 99, 100, 101, 102, 103, 104, 116, 119, 124, 127, 128, 129, 130, 131, 136, 137, 141, 142, 143, 145, 146, 147, 153, 154, 155, 156, and 184. In a preferred aspect, disclosed herein is an EON with at least one non-naturally occurring chemical modification, and/or comprising one or more additional non-naturally occurring chemical modifications in the ribose, linkage, or base moiety, with the proviso that the orphan nucleotide is not a cytidine comprising a 2′-OMe ribose substitution. In a preferred aspect, when the EON as disclosed herein is manufactured in a manufacturing facility or laboratory, the orphan nucleotide is a deoxynucleotide comprising a 6-amino-5-nitro-3-yl-2 (1H)-pyridone nucleobase (also referred to as a Benner's base; or Z), and the nucleotide on the −1 position in the EON is a deoxyinosine (Id). In a preferred aspect, the one or more additional modifications in the linkage moiety is each independently selected from a phosphorothioate (PS), phosphonoacetate, phosphorodithioate, methylphosphonate (MP; or MeP), sulfonylphosphoramidate, mesyl phosphoramidate (PNms), or a (1,3-dimethylimidazolidin-2-ylidene) phosphoramidate (PNdmi) internucleotide linkage. In a preferred aspect, the one or more additional modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O—, S—, or N-alkyl; —O—, S—, or N-alkenyl; —O—, S—, or N-alkynyl; —O—, S—, or N-allyl; —O-alkyl-O-alkyl; -methoxy; -aminopropoxy; -methoxyethoxy; -dimethylamino oxyethoxy; and dimethylaminoethoxyethoxy. Disclosed herein is also vector, preferably a viral vector, more preferably an adeno-associated virus (AAV) vector, comprising a nucleic acid molecule encoding an EON comprising a sequence according to any one of SEQ ID NO: 79, 80, 81, 82, 83, 84, 85, 86, 87, 88, 89, 90, 91, 92, 93, 94, 95, 96, 97, 98, 99, 100, 101, 102, 103, 104, and 184, wherein the orphan nucleotide is a cytidine or a uridine, and the nucleotide at the −1 position in the EON is a guanosine. Disclosed herein is also a pharmaceutical composition comprising an EON or a vector according as disclosed herein, and a pharmaceutically acceptable carrier. The disclosure also relates to an EON, or a vector, as disclosed herein, for use in the treatment of a disorder caused by a diminished GABAergic inhibition, preferably caused by a diminished KCC2 activity. In a preferred aspect, the disorder is chronic pain or epilepsy. The disclosure also relates to a use of an EON, or a vector, as disclosed herein, in the manufacture of a medicament for the treatment of a disorder caused by a diminished GABAergic inhibition, preferably caused by a diminished KCC2 activity. In a preferred aspect, the disorder is chronic pain or epilepsy.

The disclosure also relates to a method of editing a SLC12A5 polynucleotide, the method comprising contacting the SLC12A5 polynucleotide with an EON capable of effecting an ADAR-mediated adenosine to inosine editing of a target adenosine in a codon encoding an amino acid that is associated with phosphorylation of the SLC12A5-encoded protein KCC2, thereby editing the SLC12A5 polynucleotide. The disclosure also relates to a method of treating a disorder caused by a diminished GABAergic inhibition, preferably caused by a diminished KCC2 activity, in an individual in need thereof, the method comprising contacting a SLC12A5 polynucleotide in a cell of the subject with an EON capable of effecting an ADAR-mediated adenosine to inosine editing of a target adenosine in a codon encoding an amino acid that is associated with phosphorylation of the SLC12A5-encoded protein KCC2, thereby treating the individual. In a preferred aspect, the target adenosine is a first nucleotide of the codon encoding threonine at position 1007 of the SLC12A5-encoded KCC2b isoform, or wherein the target adenosine is the first nucleotide of the codon encoding threonine at position 1030 of the SLC12A5-encoded KCC2a isoform. The disclosure also relates to a method of treating a disorder caused by a diminished GABAergic inhibition, preferably caused by a diminished KCC2 activity, the method comprising administering to an individual in need thereof a therapeutically effective amount of an EON, a vector, or a pharmaceutical composition as disclosed herein. In a preferred aspect, the disorder is chronic pain or epilepsy.

The disclosure also relates to a method of deaminating a target adenosine in an SLC12A5 pre-mRNA or mRNA molecule in a cell, the method comprising the steps of: (i) providing the cell with an EON as disclosed herein; (ii) allowing uptake by the cell of the EON; (iii) allowing annealing of the EON to the SLC12A5 pre-mRNA or mRNA molecule; (iv) allowing an endogenous ADAR enzyme (such as ADAR1 or ADAR2) to deaminate the target adenosine in the target RNA molecule to an inosine; and optionally (v) identifying the presence of the inosine in the target RNA molecule. In a preferred aspect, the target adenosine is a first nucleotide of the codon encoding threonine at position 1007 of the SLC12A5-encoded KCC2b isoform, and in an alternative aspect the target adenosine is the first nucleotide of the codon encoding threonine at position 1030 of the SLC12A5-encoded KCC2a isoform. In a preferred aspect, step (v) comprises: a) determining the sequence of the SLC12A5 pre-mRNA or mRNA molecule; b) assessing the presence of an SLC12A5-encoded KCC2 protein with a lower phosphorylation rate, preferably assessing the presence of KCC2 protein with an absent phosphorylation at position 1007 in the KCC2b isoform (or at position 1030 in the KCC2a isoform); or c) using a functional read-out, preferably assessing the level of GABAergic inhibition in the cell.

The present disclosure also relates to a nucleic acid molecule for editing a target adenosine in a human SLC12A5 pre-mRNA or mRNA molecule, wherein the target region is SEQ ID NO: 105, and wherein the target adenosine is the first nucleotide of the codon encoding threonine at position 1007 of the SLC12A5-encoded KCC2b isoform (or alternatively at position 1030 in the KCC2a isoform). In a preferred aspect, the nucleic acid molecule is selected from the group consisting of SEQ ID NOS: 79, 80, 81, 82, 83, 84, 85, 86, 87, 88, 89, 90, 91, 92, 93, 94, 95, 96, 97, 98, 99, 100, 101, 102, 103, 104, and 184, that preferably comprises at least one non-naturally occurring chemical modification, and/or comprising one or more additional non-naturally occurring chemical modifications in a ribose, linkage or base moiety, with the proviso that the orphan nucleotide, which is the nucleotide in the nucleic acid that is directly opposite a target adenosine in the target region, is not a cytidine comprising a 2′-OMe ribose substitution. In a preferred aspect, and as outlined above, the one or more additional modifications in the linkage moiety is each independently selected from a PS, phosphonoacetate, phosphorodithioate, MP, sulfonylphosphoramidate, PNms, or PNdmi internucleotide linkage. The disclosure also relates to a vector comprising a nucleotide sequence encoding the nucleic acid molecule as disclosed herein, wherein the orphan nucleotide is a cytidine or a uridine, and the nucleotide at the −1 position in the EON is a guanosine.

Disclosed herein are EONs that can mediate RNA editing of a target adenosine in the human SLC12A5 transcript (pre-mRNA and/or mRNA), through which the resulting KCC2 protein is mutated at a particular phosphorylation site. The absence of phosphorylation at this site increases the activity of the resulting (mutant) KCC2 protein. In a preferred aspect, the EON causes the deamination of the adenosine at position 3149 of the wild-type SLC12A5 mRNA (see NCBI Ref. Seq. No. NM_020708.5), encoding the KCC2b isoform, thereby generating an inosine. Likewise, the adenosine at position 3168 of the wild-type SLC12A5 mRNA encoding the KCC2a isoform can be targeted (see NCBI Ref. Seq. No. NM_001134771.2). In detail, the ACC codon encoding threonine (wild-type form) at amino acid position 1007 (KCC2b) is converted to an ICC codon, which is read as GCC that encodes alanine (mutant form). In another embodiment, an EON herein causes the deamination of another adenosine present in the SLC12A5 transcript, which may be any adenosine that, when deaminated into an inosine, results in a KCC2 protein with a gain-of-function. In another embodiment, an EON herein causes the deamination of an adenosine present in a mutant SLC12A5 transcript, especially when that mutation is a G>A mutation, which causes the resulting KCC2 protein to have a loss-of-function, or which makes it inactive in one or more of its functional properties, preferably regarding Cl-efflux. Other mutations may be present in the SLC12A5 gene (and transcript), that may be targeted through RNA editing thereby restoring the normal KCC2 function.

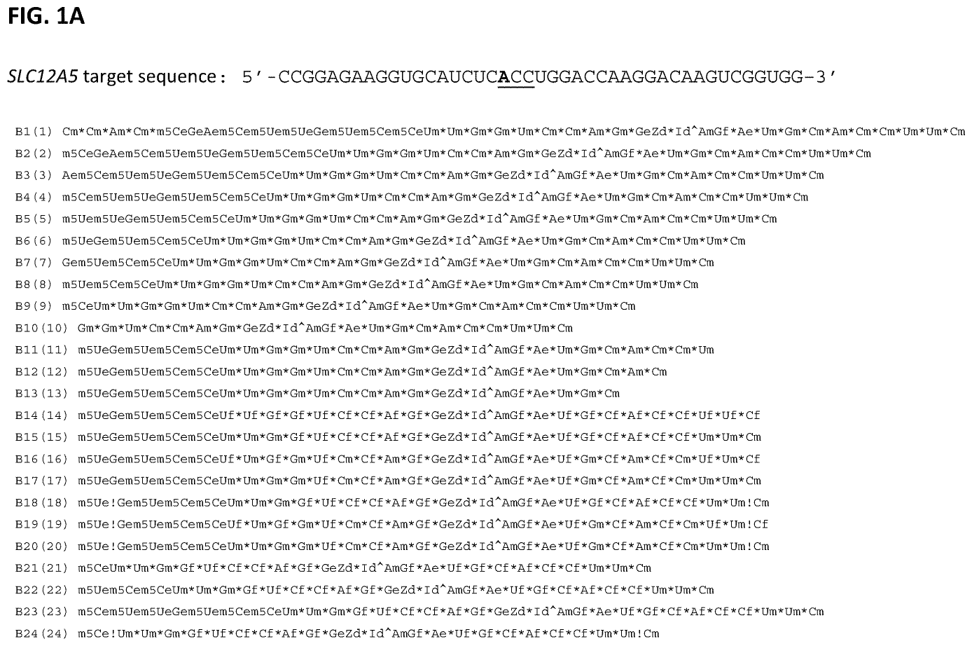

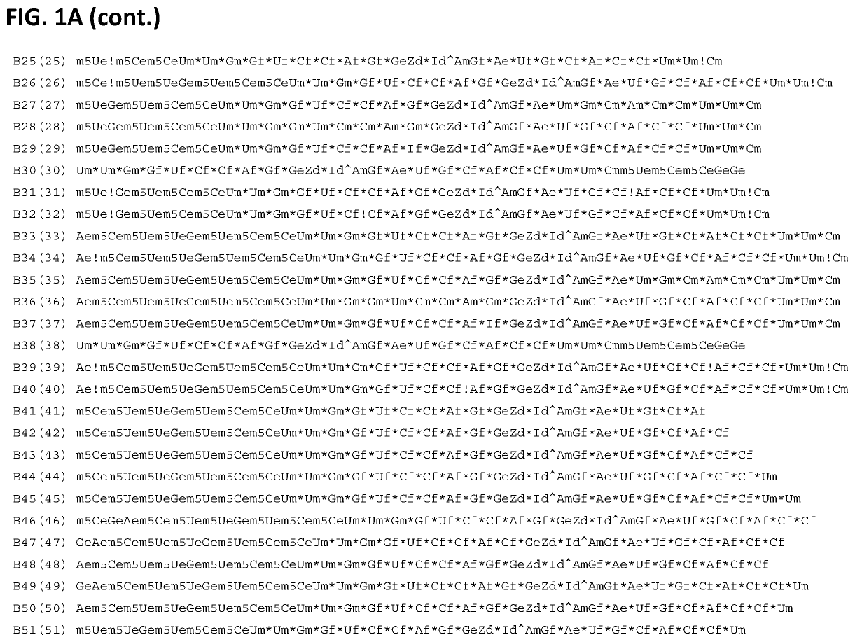

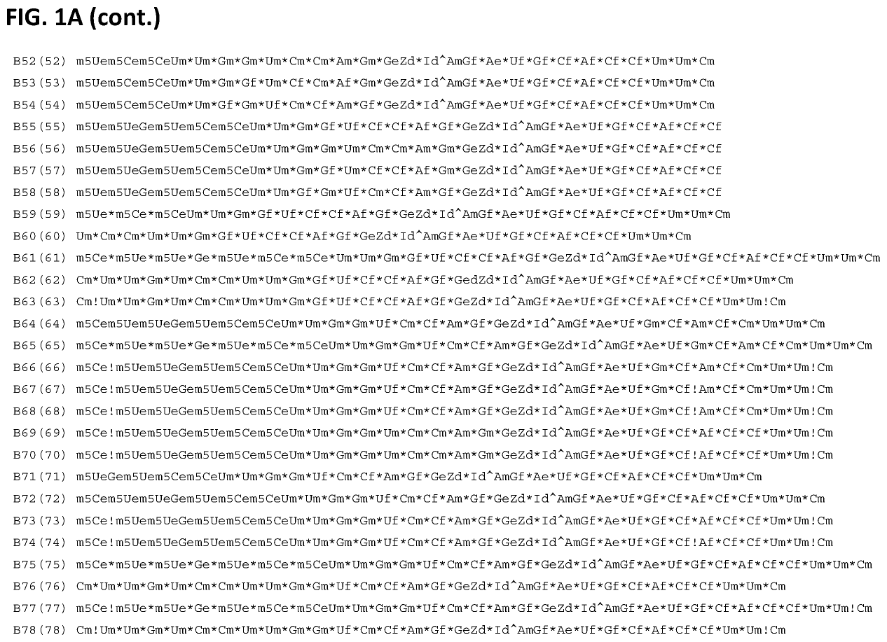

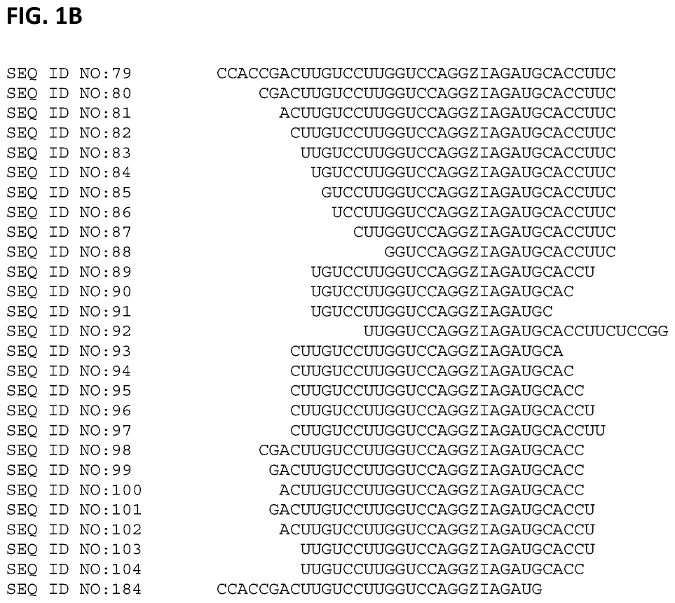

Although in a preferred embodiment, the EON herein is a single-stranded oligonucleotide comprising an orphan nucleotide as defined above, wherein the orphan nucleotide is chemically modified as disclosed herein, and wherein the remainder of the oligonucleotide is also chemically modified to prevent it from nuclease breakdown also as disclosed herein, in another embodiment, the disclosure relates to any kind of oligonucleotide or heteroduplex oligonucleotide complex, that may or may not be bound to hairpin structures (internally or at the terminal end(s)), that may be bound to ADAR or catalytic domains thereof, or wherein the oligonucleotide is expressed through a vector, such as an adeno-associated virus (AAV), or wherein the oligonucleotide is in a circular format. It is to be understood that any kind of oligonucleotide-based RNA editing is encompassed by the disclosure if it relates to the deamination of an adenosine in the SLC12A5 transcript, preferably the adenosine at position 1 in the codon encoding threonine at position 1007 in KCC2b. In a preferred aspect, an EON herein is a ‘naked’ oligonucleotide, comprising a variety of chemical modifications in the ribose sugar, the base, and/or the internucleoside linkage of one or more of the nucleotides within the sequence, that can hybridize to the SLC12A5 transcript or a part thereof that includes the target adenosine, and can recruit endogenous ADAR for the deamination of the target adenosine. The endogenous ADAR enzyme is preferably human ADAR1 or ADAR2. The cell is preferably a human neuronal cell. The SLC12A5 transcript molecule is preferably a pre-mRNA or an mRNA molecule. The EON herein preferably targets an adenosine for deamination that causes a gain-of-function of the KCC2 protein. Although several mutations are known that cause a dysfunction of the KCC2 protein, a preferred adenosine that is targeted through the EONs as disclosed herein is an adenosine that is in a codon that encodes a phosphorylation site in KCC2, wherein the resulting codon (after deamination of the adenosine) is no longer a phosphorylation site. Loss of phosphorylation of this site in KCC2 increases its GABAergic inhibitory activity, thereby lowering abnormal neuronal activation (e.g., causing chronic neurological pain) and synchronization that underlies seizures. A preferred threonine that is amended through editing of the SLC12A5 transcript is the threonine at position 1007 in the KCC2b isoform, which will be amended to an alanine that can no longer get phosphorylated. The EONs herein are capable of bringing about the deamination of the adenosine in the ACC codon encoding threonine, thereby generating an ICC codon, which is translated to alanine because the codon is read as GCC. In one embodiment, the EON herein comprises, or consists of, the sequence of any one of the EON sequences depicted in FIG. 1A (SEQ ID NO:1 to 78). In an embodiment, the EON herein comprises or is entirely composed of nucleotides, each carrying the chemical modifications referred to in FIG. 1A.

In one embodiment, the orphan nucleotide is a cytidine, a deoxycytidine, a cytidine analog (such as a nucleoside comprising a Benner's base), a uridine, a deoxyuridine, or a uridine analog (such as iso-uridine). In one embodiment, the EON comprises at least one mismatch with the (overlapping) sequence of the target transcript molecule. When the orphan nucleotide is uridine, then the EON does not necessarily comprise a mismatch. Mismatches may be introduced in other parts of the EON, where required, as long as the EON is capable of hybridizing under natural conditions to the target transcript.

In one embodiment, an EON herein comprises at least one nucleotide comprising one or more non-naturally occurring chemical modifications, or one or more additional non-naturally occurring chemical modifications, in the ribose, linkage, or base moiety, with the proviso that the orphan nucleotide is not a cytidine comprising a 2′-OMe ribose substitution. In an embodiment, the EON herein comprises one or more mismatches, wobbles, or bulges, wherein a single mismatch may be present when the target adenosine has an opposite cytidine, or a uridine analog (that not fully matches in comparison to a uridine) in the EON. If the orphan nucleotide is a cytidine, that cytidine does not comprise a 2′-OMe ribose substitution, as indicated above. Preferably, also when the orphan nucleotide is different from a cytidine, it also does not comprise a 2′-OMe ribose substitution if it hinders deamination by the ADAR enzyme.

Disclosed herein is a vector, preferably a viral vector, more preferably an adeno-associated virus (AAV) vector, comprising a nucleic acid molecule encoding an EON herein. When the EON is delivered through the means of a viral vector or a plasmid vector, the produced EON in the cell does not have chemical modifications. Further disclosed herein is a pharmaceutical composition comprising an EON as disclosed herein, or a viral vector or plasmid vector as disclosed herein, and a pharmaceutically acceptable carrier.

In an embodiment, disclosed herein is an EON, a vector, or a pharmaceutical composition for use in the treatment of a subject in need thereof, wherein the subject suffers from a disorder, wherein the KCC2 inhibitory activity is lowered or absent, either through a loss-of-function mutation, lowered expression of the transcript and/or protein, or through (potentially increased rates of) post-translational modifications such as activity-inhibiting phosphorylation of certain sites in the (wild-type) protein. In an embodiment, disclosed herein is an EON or a vector in the manufacture of a medicament for the treatment of a disorder in a subject, wherein the KCC2 inhibitory activity in neuronal cells is lowered or absent, either through a loss-of-function mutation, lowered expression of the transcript and/or protein, or through (potentially increased rates of) post-translational modifications such as activity-inhibiting phosphorylation of certain sites in the (wild-type) protein.

In an embodiment, disclosed is a method of editing an SLC12A5 polynucleotide, the method comprising contacting the SLC12A5 polynucleotide with an EON capable of effecting an ADAR-mediated adenosine-to-inosine alteration of an adenosine in a codon encoding threonine that is associated with phosphorylation of the resulting protein KCC2, thereby editing the SLC12A5 polynucleotide. The SLC12A5 polynucleotide is preferably a pre-mRNA or mRNA nucleic acid molecule. In an embodiment, disclosed is a method of treating a disorder caused by a lowered or diminished KCC2 activity (in its GABAergic inhibitory action, especially in an activity wherein the Cl-efflux from neuronal cells is at a too low level), or a disorder caused by a loss-of-function mutant of KCC2, the method comprising contacting a SLC12A5 polynucleotide in a cell of the subject with an EON capable of effecting an ADAR-mediated adenosine to inosine alteration of an adenosine in a codon coding for a phosphorylation site, preferably the threonine at position 1007 in KCC2b, thereby treating the patient. In an embodiment, disclosed is a method of treating epilepsy or pathological, neurological (chronic) pain in a human subject in need thereof, the method comprising administering to the subject a therapeutically effective amount of an EON, a vector, or a pharmaceutical composition as disclosed herein.

### Definitions

The term ‘nucleoside’ refers to the nucleobase linked to the (deoxy) ribosyl sugar, without phosphate groups. A ‘nucleotide’ is composed of a nucleoside and one or more phosphate groups. The term ‘nucleotide’ thus refers to the respective nucleobase-(deoxy) ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a phosphodiester, phosphonoacetate, phosphotriester, PS, phosphoro(di)thioate, MP, methyl thiophosphonate, phosphoramidate linkages, and the like. Sometimes the terms adenosine and adenine, guanosine and guanine, cytidine and cytosine, uracil and uridine, thymine and thymidine/uridine, inosine, and hypoxanthine, are used interchangeably to refer to the corresponding nucleobase on the one hand, and the nucleoside or nucleotide on the other. Thymine (T) is also known as 5-methyluracil (m⁵U) and is a uracil (U) derivative; thymine, 5-methyluracil and uracil can be interchanged throughout the document text. Likewise, thymidine is also known as 5-methyluridine and is a uridine derivative; thymidine, 5-methyluridine and uridine can be interchanged throughout the document text. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated herein, a nucleotide is a nucleoside plus one or more phosphate groups. The terms ‘ribonucleoside’ and ‘deoxyribonucleoside’, or ‘ribose’ and ‘deoxyribose’ are as used in the art.

Whenever reference is made to an oligonucleotide, oligo, ON, ASO, oligonucleotide composition, antisense oligonucleotide, AON, (RNA) editing oligonucleotide, EON, and RNA (antisense) oligonucleotide, both oligoribonucleotides and deoxyoligoribonucleotides are meant unless the context dictates otherwise. Potentially the oligonucleotide may completely lack RNA or DNA nucleotides (as they appear in nature) and may consist completely of modified nucleotides. Whenever reference is made to an ‘oligoribonucleotide’ it may comprise the bases A, G, C, U, or I. Whenever reference is made to a ‘deoxyoligoribonucleotide’ it may comprise the bases A, G, C, T, or I. However, an EON herein may comprise a mix of ribonucleosides and deoxyribonucleosides. When a deoxyribonucleotide is used, hence without a modification at the 2′ position of the sugar, the nucleotide is often abbreviated to dA. dC, dG or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included. Whenever reference is made to adenine, N6-methyladenine, 8-oxo-adenine, 2,6-diaminopurine and 7-methyladenine are included. Whenever reference is made to uracil, dihydrouracil, iso-uracil, N3-glycosylated uracil, pseudouracil, 5-methyluracil, N1-methylpseudouracil, 4-thiouracil and 5-hydroxymethyluracil are included. Whenever reference is made to guanine, 1-methylguanine, 7-methylguanosine, N2,N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-OMe, are included, as well as other modifications, including 2′-4′ bridged variants. Whenever reference is made to oligonucleotides, linkages between two mononucleotides may be phosphodiester linkages as well as modifications thereof, including, phosphonoacetate, phosphotriester, PS, phosphoro(di)thioate, MP, phosphoramidate linkers, phosphoryl guanidine, thiophosphoryl guanidine, sulfono phosphoramidate and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x±10%.

The word ‘substantially’ does not exclude ‘completely’, e.g., a composition which is ‘substantially free from Y’ may be completely free from Y. Where relevant, the word ‘substantially’ may be omitted from the definition of the invention.

The term ‘complementary’ as used herein refers to the fact that the EON hybridizes under physiological conditions to a second nucleic acid strand (for instance when the oligonucleotide as a first nucleic acid strand (=guide oligonucleotide) forms a heteroduplex RNA editing oligonucleotide complex (HEON) with another complementary nucleic acid strand), or when it forms a ds complex with the target RNA sequence. The term does not necessarily mean that each nucleotide in a nucleic acid strand has a perfect pairing with its opposite nucleotide in the opposite sequence. In other words, while an EON may be complementary to a target sequence, there may be mismatches, wobbles and/or bulges between the oligonucleotide and the target sequence, while under physiological conditions that EON still hybridizes to the target sequence such that the cellular RNA editing enzymes can edit the target adenosine. The term ‘substantially complementary’ therefore also means that despite the presence of the mismatches, wobbles, and/or bulges, the EON has enough matching nucleotides between the EON and target sequence that under physiological conditions the EON hybridizes to the target RNA. As shown herein, an EON may be complementary, but may also comprise one or more mismatches, wobbles and/or bulges with the target sequence, if under physiological conditions the EON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments an EON as disclosed herein comprises fewer than four mismatches with the target sequence, for example 0, 1 or 2 mismatches. ‘Wobble’ base pairs are G-U, I-U, I-A, and I-C base pairs. Although a G:G pairing would be considered a mismatch, that does not necessarily mean that the interaction is unstable, which means that the term ‘mismatch’ may be somewhat outdated based on the current disclosure where a Hoogsteen base-pairing may be seen as a mismatch based on the origin of the nucleotide but still be relatively stable. An isolated G:G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity.

An EON (and the complementary nucleic acid strand when two oligonucleotides form a HEON) as disclosed herein may be chemically modified almost in its entirety, for example by providing nucleotides with a ribose sugar moiety carrying a 2′-OMe substitution, a 2′-F substitution, or a 2′-O-methoxyethyl(2′-MOE) substitution. The orphan nucleotide in the EON is preferably a cytidine or analog thereof (such as a nucleotide carrying a Benner's base), or a uridine or analog thereof (such as iso-uridine), and/or in one embodiment comprises a diF modification at the 2′ position of the sugar, in another embodiment comprises a deoxyribose (2′-H, DNA), and in yet a further embodiment, at least one and in another embodiment both the two neighbouring nucleotides flanking the orphan nucleotide do not comprise a 2′-OMe modification. Complete modification wherein all nucleotides of the oligonucleotide hold a 2′-OMe modification, with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group (at least when there are no other chemical substitutions or modifications within the nucleotide), or by providing a guanine or adenine as opposing base, as these two nucleobases are also able to reduce editing of the opposing adenosine.

Various chemistries and modifications are known in the field of oligonucleotides that can be readily used in accordance with the disclosure. The regular internucleoside linkages between the nucleotides may be altered by mono- or di-thioation of the phosphodiester bonds to yield PS esters or phosphorodithioate esters, respectively. Other modifications of internucleoside linkages are possible, including amidation and peptide linkers.

In an embodiment, the EON herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. Notably, when the EON is delivered through a (viral) vector, the length may increase as being longer than 60 nucleotides. However, when the EON is to be delivered as is, without a vector, also referred to as a ‘naked form’, the length of the EON is 15 to 60 nucleotides to reduce the risk of degradation. Furthermore, in a naked form, the EON is preferably chemically modified as outlined herein to lower the risk of degradation.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably helps to position the dsRNA binding domains in a way that has not been clearly defined yet. Additionally, the deamination reaction itself can be enhanced by providing an oligonucleotide that comprises a mismatch opposite the adenosine to be edited. Following the instructions in the present application, those of skill in the art will be capable of designing the complementary portion of the oligonucleotide according to their needs.

The RNA editing proteins present in the cell that are of most interest to be used with an EON as disclosed herein are human ADAR1 and ADAR2. It will be understood by a person having ordinary skill in the art that the extent to which the editing entities inside the cell are redirected to other target sites may be regulated by varying the affinity of the first nucleic acid strand for the recognition domain of the editing molecule. The exact modification may be determined through some trial and error and/or through computational methods based on structural interactions between the EON and the recognition domain of the editing molecule. In addition, or alternatively, the degree of recruiting and redirecting the editing entity resident in the cell may be regulated by the dosing and the dosing regimen of the EON. This is something to be determined by the experimenter (in vitro) or the clinician, usually in phase I and/or II clinical trials.

The disclosure concerns the modification of target RNA sequences in eukaryotic, preferably metazoan, more preferably mammalian, even more preferably human cells, and most preferably human neuronal cells. The EONs, vectors and pharmaceutical compositions herein are particularly suitable for modifying RNA sequences in cells and tissues in which KCC2 is expressed and wherein that protein acts. Because KCC2 is exclusively produced and has an important role in neurons in chloride extrusion, the preferred target cell for the EONs herein is neuronal. The target cell can be located in vitro, ex vivo or in vivo. One advantage of the EONs herein is that they can be used with cells in situ in a living organism, but they can also be used with cells in culture. In some embodiments, cells are treated ex vivo and are then introduced into a living organism (e.g., re-introduced into an organism from whom they were originally derived). The EONs herein can also be used to edit target RNA sequences in cells from a transplant or within a so-called organoid, e.g., a brain tissue organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant.

Without wishing to be bound by theory, RNA editing through human ADAR is thought to take place on primary transcripts in the nucleus, during transcription or splicing, or in the cytoplasm, where, e.g., mature mRNA, miRNA or ncRNA can be edited.

It should be clear, that targeted editing as described herein can be applied to any adenosine within the SLC12A5 transcript if the deamination of the adenosine results in an increase or restoration of KCC2 protein function. As outlined herein, however, it is preferred to target the first adenosine that is present in the codon encoding threonine at position 1007 of the mature KCC2b splice variant.

Generally spoken, RNA editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present disclosure. Hence, an EON as disclosed herein may mediate the RNA editing of any target adenosine in the SLC12A5 transcript which results in improvement or restoration of the KCC2 protein function. The disclosure opens a whole new field of treating pathological pain (such as chronic pain) and epilepsy, using genetic editing techniques.

The amount of EON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change, or a change in (the level of, or activity of) a specified biomarker. It is possible that higher doses of EONs could compete for binding to an ADAR within a cell, thereby depleting the amount of the entity, which is free to take part in RNA editing, but routine dosing trials will reveal any such effects for a given EON and a given target.

One suitable trial technique involves delivering the EON to cell lines, or a test organism and then taking biopsy samples at various time points thereafter. The sequence of the target RNA can be assessed in the biopsy sample and the proportion of cells having the modification can easily be followed. Also, a suitable biomarker that can be used following the present disclosure is to detect phosphorylation of the threonine at position 1007, and by assessing the function/activity of the KCC2 protein in a particular subject, before and after treatment, or with or without treating the subject with an EON or vector as disclosed herein. After this trial has been performed once then the knowledge can be retained, and future delivery can be performed without needing to take biopsy samples. A method as disclosed herein can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, as indicated above, the change may be assessed on the function of the protein, or instance by measuring thallium transport capacity of KCC2. The transport of thallium, a surrogate of potassium, is directly proportional to the number of active KCC2 potassium transporters. Thallium transport can then be detected by introducing a highly sensitive thallium indicator dye, before, during, and/or after treatment or assessing any other potential marker, which measurements are preferably performed in vitro on samples obtained from the treated subject.

After RNA editing has occurred in a cell, the modified RNA can become diluted over time, for example due to cell division, limited half-life of the edited RNAs, etc. Thus, in practical therapeutic terms a method as disclosed herein may involve repeated delivery of an EON until enough target RNAs have been modified to provide a tangible benefit to the patient and/or to maintain the benefits over time.

EONs herein are particularly suitable for therapeutic use, and so the disclosure also relates to a pharmaceutical composition comprising an EON herein, or a vector or plasmid encoding an EON herein, and a pharmaceutically acceptable carrier. In some embodiments, the pharmaceutically acceptable carrier can simply be a saline solution. This can usefully be isotonic or hypotonic, particularly for pulmonary delivery. The disclosure also provides a delivery device (e.g., a syringe) that includes a pharmaceutical composition herein.

The disclosure also provides an EON herein for use in a method for introducing a phosphorylation mutation in a target SLC12A5 RNA sequence in a mammalian, preferably a human neuronal cell, as described herein. Similarly, the disclosure provides the use of an EON herein in the manufacture of a medicament for making a change in a target SLC12A5 RNA sequence in a mammalian, preferably a human neuronal cell, as described herein, and thereby treating, preventing, or ameliorating diseases related to diminished GABAergic inhibition, such as those resulting from lowered KCC2 activity.

The EONs herein are suitably administrated in aqueous solution, e.g. saline, artificial cerebrospinal fluid, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intradermally, intramuscularly, intra-tracheally, intra-peritoneally, intrarectally, intrathecally, intra-cisterna magna, parenterally, and the like. Administration may be in solid form, in the form of a powder, a pill, a gel, a solution, a slow-release formulation, or in any other form compatible with pharmaceutical use in humans.

In one embodiment, a method herein comprises the steps of administering to the subject an EON or pharmaceutical composition herein, allowing the formation of a ds nucleic acid complex of the EON with its specific complementary target nucleic acid molecule in a cell in the subject; allowing the engagement of an endogenous present adenosine deaminating enzyme, such as ADAR2; and allowing the enzyme to deaminate the target adenosine in the target nucleic target molecule to an inosine, thereby alleviating, preventing or ameliorating the disease related to lowered GABAergic inhibition. The diseases that may be treated according to this method are preferably, but not limited to, the CNS diseases listed herein, and any other disease in which deamination of an adenosine in SLC12A5 transcripts would restore the KCC2 protein's function in an individual in need thereof.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof. RNA editing enzymes known in the art, for which oligonucleotide constructs according to the present disclosure may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 110 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and EONs as disclosed herein are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

### Chemical Modifications

All chemical modifications listed below that may be used in the EONs herein may also be used for a sense strand that is complementary to an EON, when the EON and the complementary strand form a so-called HEON complex, as described in GB Patent Application No. 2215614.5 (unpublished), except that the opposite sense strand does not have an orphan nucleotide. Hence, the modification related to the orphan nucleotide relate only to the EONs herein, but all other modifications relate to the EONs herein and any (protecting) sense oligonucleotide that may be used together with the EONs in a pharmaceutical product. This includes the use of hydrophobic moieties (such as tocopherol and cholesterol) and cell-specific ligands (such as GalNAc moieties), that have also been described herein, and in detail in GB Patent Application No. 2215614.5 (unpublished), which may either be bound to the EON or its opposite strand, or both.

The internucleoside linkages in the oligonucleotides herein may comprise one or more naturally occurring internucleoside linkages and/or modified internucleoside linkages. Without limitations, at least one, at least two, or at least three internucleoside linkages from a 5′ and/or 3′ end of the EON are preferably modified internucleoside linkages. A preferred modified internucleoside linkage is a PS linkage. In one embodiment, all internucleoside linkages of the EON are modified internucleoside linkages. In one embodiment, the EON comprises a PNdmi linkage linking the most terminal nucleoside at the 5′ and/or 3′ end, and the one before last nucleoside at each of these ends, respectively. A PNdmi linkage as preferably used in the EONs herein has the structure of formula (I):

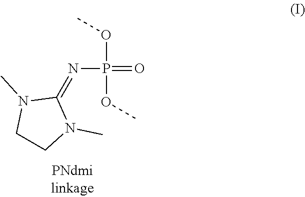

A common limiting factor in oligonucleotide-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-O-methyl (often abbreviated to 2′-OMe or 2′-O-Me), 2′-F and 2′-O-methoxyethyl (often also referred to as 2′-methoxyethoxy, or 2′-MOE) modifications of the sugar and the use of PS linkages between nucleosides. Intl. Patent Application No. WO2020/201406 discloses the use of MP linkage modifications at certain positions surrounding the orphan nucleotide in the first nucleic acid strand. The ribose 2′ groups in all nucleotides of the EON, except for the ribose sugar moiety of the orphan nucleotide that has certain limitations in respect of compatibility with RNA editing, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in the EON that comprises no other chemical modifications to the ribose sugar, the base, or the linkage preferably does not carry a 2′-OMe or 2′-MOE substitution but may carry a 2′-F, a 2′,2′-difluoro (diF), or 2′-ara-F (FANA) substitution or may be DNA. GB 2214347.3 (unpublished) describes the modification of the 2′ position of the ribose sugar moiety of the orphan nucleotide by a 2′,2′-disubstituted substitution such as diF, which is also applicable here. The 2′-4′ linkage can be selected from many linkers known in the art, such as a methylene linker, amide linker, or constrained ethyl linker (cEt).

The disclosure relates to an EON for use in the deamination of a target nucleotide (preferably adenosine) in a target RNA, wherein the EON is complementary to a stretch of nucleotides in the target RNA that includes the target adenosine, wherein the nucleotide in the first nucleic acid strand that is directly opposite the target nucleotide is the orphan nucleotide, and when the target nucleotide is an adenosine the orphan nucleotide comprises preferably a base or modified base or base analogue with a NH moiety at the position similar to the ring nitrogen (e.g., Benner's base Z). The nucleotide numbering in the EON is such that the orphan nucleotide is number 0 and the nucleotide 5′ from the orphan nucleotide is number +1. Counting is further positively (+) incremented towards the 5′ end and negatively (−) incremented towards the 3′ end, wherein the first nucleotide 3′ from the orphan nucleotide is number −1. The internucleoside linkage numbering in the EON is such that linkage number 0 is the linkage 5′ from the orphan nucleotide, and the linkage positions in the oligonucleotide are positively (+) incremented towards the 5′ end and negatively (−) incremented towards the 3′ end.

Preferably, the EON comprises one or more (chirally pure or chirally mixed) PS linkages. In one embodiment, the PS linkages connect the terminal 3, 4, 5, 6, 7, or 8 nucleotides on each end of the first nucleic acid strand. In one embodiment, the EON comprises one of more phosphoramidate (PN) linkages. In one embodiment, a PN linkage connects the terminal two nucleotides on each end of the EON.

A nucleoside in the EON may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded substrate configurations. The skilled person appreciates that when the nucleotide has a natural ribose moiety, it may still be non-naturally modified in the base and/or the linkage.

In addition to the specific preferred chemical modifications at certain positions in compounds herein, which may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O— (substituted)alkyl such as 2′-OMe, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino) propyl), 2′-O-(2-amino)ethyl, 2′-O-(2-(dimethylamino)ethyl); 2′-deoxy (DNA); 2′-O-(haloalkyl) methyl such as 2′-O-(2-chloroethoxy) methyl (MCEM), 2′-O-(2,2-dichloroethoxy) methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an a-LNA monomer, an a-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-l-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

The base sequence of the EON herein is complementary to part of the base sequence of a target SLC12A5 transcription product that includes at least the target adenosine that is to be deaminated to an inosine, and therefore can anneal (or hybridize) to the target transcription product. The complementarity of a base sequence can be determined by using a BLAST program or the like. Those skilled in the art can easily determine the conditions (temperature, salt concentration, and the like) under which two strands can be hybridized, taking into consideration the complementarity between the strands.

An EON herein, in contrast to what has been described for gapmers and their relation towards RNase breakdown and the use of such gapmers in double-stranded complexes (see for instance EP Patent Application Publication No. 3954395 A1), does not comprise a stretch of DNA nucleotides that would make a target sequence (or a sense nucleic acid strand) a target for RNase-mediated breakdown. In one embodiment, the EON does not comprise four or more consecutive DNA nucleotides anywhere within its sequence. In an embodiment, the EON is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the EON may be DNA, but also that there is no stretch of four or more consecutive DNA nucleotides within the EON. Hence, an EON herein is not a gapmer. A gapmer reduces the expression of a target transcript but does not produce RNA editing of a specified adenosine within the target transcript. A gapmer is in principle a ss nucleic acid consisting of a central region (DNA gap region with at least four consecutive deoxyribonucleotides) and wing regions positioned directly at the 5′ end (5′ wing region) and the 3′ end (3′ wing region) thereof. In contrast, the EON herein may be any oligonucleotide that produces an RNA editing effect in which a target adenosine in a target RNA molecule is deaminated to an inosine, and accordingly is resistant to RNase-mediated breakdown as much as possible to yield this effect. The purpose of the EONs herein is to increase KCC2 activity, not to reduce it, for instance by causing a breakdown of the KCC2-encoding transcript molecules.

In one embodiment, the EON, or the sense strand to which it may be annealed before entering a target cell, is bound to, or associated with a blood-brain-barrier shuttle, or a hydrophobic moiety, such as palmityl or an analog thereof, cholesterol or analog thereof, or tocopherol or analog thereof. It is preferably bound to the 5′ terminus. In case a hydrophobic moiety is bound to the 5′ terminus as well as to the 3′ terminus, such hydrophobic moieties may the same or different. The hydrophobic moiety bound to the oligonucleotide may be bound directly, or indirectly mediated by another substance. When the hydrophobic moiety is bound directly, it is sufficient if the moiety is bound via a covalent bond, an ionic bond, a hydrogen bond, or the like. When the hydrophobic moiety is bound indirectly, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations). Examples of a cleavable linker comprise, but is not limited to, an amide, an ester, one or both esters of a phosphodiester, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a PS linkage, modified or unmodified deoxyribonucleosides linked by a PS linkage, a spacer connected through a PS bond and a linker consisting of modified or unmodified ribonucleosides. There is no restriction on the chain length, when a linker is a nucleic acid such as DNA, or an oligonucleotide. However, it may be usually from 2 to 20 bases in length, from 3 to 10 bases in length, or from 4 to 6 bases in length. There is no restriction on the length or composition of a spacer that is connects the ligand and the oligonucleotide, and may include for example ethylene glycol, TEG, HEG, alkyl chains, propyl, 6-aminohexyl, or dodecyl.

The disclosure also relates to a pharmaceutical composition comprising an EON herein, and further comprising a pharmaceutically acceptable carrier and/or other additive and may be dissolved in a pharmaceutically acceptable organic solvent, or the like. Dosage forms in which the EON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient.

In one embodiment, the EON comprises at least one nucleotide with a sugar moiety that comprises a 2′-OMe modification. In one embodiment, the EON comprises at least one nucleotide with a sugar moiety that comprises a 2′-MOE modification. In one embodiment, the EON comprises at least one nucleotide with a sugar moiety that comprises a 2′-F modification. In one embodiment, the orphan nucleotide carries a 2′-H in the sugar moiety and is therefore referred to as a DNA nucleotide, even though additional modifications may exist in its base and/or linkage to its neighbouring nucleosides. In one embodiment, the orphan nucleotide carries a 2′-F in the sugar moiety. In one embodiment, the orphan nucleotide carries a diF substitution in the sugar moiety. In one embodiment, the orphan nucleotide carries a 2′-F and a 2′-C-methyl in the sugar moiety. In one embodiment, the orphan nucleotide comprises a 2′-F in the arabinose configuration (FANA) in the sugar moiety. In one embodiment, the EON is an antisense oligonucleotide (sometimes also generally abbreviated to “ASO”) that can form a double-stranded nucleic acid complex with a target RNA molecule, wherein the double-stranded nucleic acid complex can recruit an adenosine deaminating enzyme for deamination of a target adenosine in the target SLC12A5 RNA molecule, wherein the nucleotide in the EON that is opposite the target adenosine is the orphan nucleotide, and wherein the orphan nucleotide has the structure of formula (II):

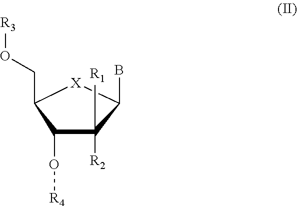

wherein: X is O, NH, OCH₂, CH₂, Se, or S; B is a nitrogenous base selected from the group consisting of: cytosine, uracil, iso-uracil, N3-glycosylated uracil, pseudoisocytosine, 8-oxo-adenine, and 6-amino-5-nitro-2(1H)-pyridone; R₁ and R₂ are both selected, independently, from H, OH, F or CH₃; R₃ is the part of the EON that is 5′ of the orphan nucleotide, consisting of 7 to 30 nucleotides; and R₄ is the part of the EON that is 3′ of the orphan nucleotide, consisting of 4 to 25 nucleotides. The nucleotide 3′ and/or 5′ from the orphan nucleotide may be DNA, more preferably the nucleotide at the 3′ (position-1).

In one embodiment, the first nucleic acid strand comprises at least one MP internucleoside linkage according to the structure of formula (III):

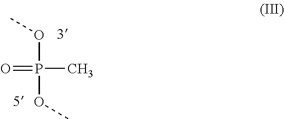

A preferred position for an MP linkage in an EON as disclosed herein is linkage position −2 (for example as shown for all EONs depicted in FIG. 1A, wherein Zd is the orphan nucleotide and Id is the nucleotide at position −1), thereby connecting the nucleoside at position −1 with the nucleoside at position −2, although other positions for MP linkages are not explicitly excluded.

An EON as disclosed herein may also comprise one or more linkage modifications according to the structure of the following formula (IV):

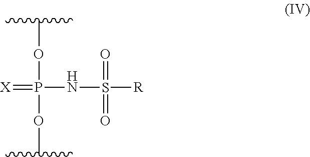

wherein:
X=O or S; and
R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X=O and R=methyl and the linkage modification is referred to as “mesyl phosphoramidate”, “MsPA” or “PNms”. In one embodiment, a PNms linkage is used instead of the MP and/or PNdmi linkages.

In an embodiment, the EON as disclosed herein comprises an internucleoside linkage of the structure of formula (IV), wherein X=O and R=CH₃, which linkage is generally referred to herein as a PNms linkage (mesyl phosphoramidate). In other preferred aspects, R equals one of the following structures (a), (b), (c), (d), (e), (f), (g), (h), or (i):

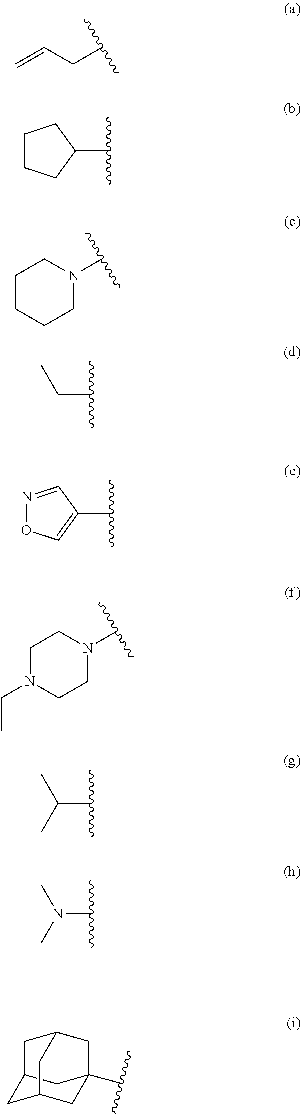

Other internucleoside linkages that may be used in the EONs of the present disclosure are those that are disclosed in WO2023/278589.

In one embodiment, the EON comprises at least one nucleotide with a sugar moiety that comprises a 2′-fluoro (2′-F) modification. A preferred position for the nucleotide that carries a 2′-F modification is position −3 in the EON, which may be present in concert with an identical 2′ modification in the orphan nucleotide as discussed above.

In one embodiment, the EON comprises at least one phosphonoacetate or phosphonoacetamide internucleoside linkage.

In one embodiment, the EON comprises at least one nucleotide comprising a locked nucleic acid (LNA) ribose modification, or an unlocked nucleic acid (UNA) ribose modification. In an embodiment, the EON comprises at least one nucleotide comprising a threose nucleic acid (TNA) ribose modification.

The skilled person knows that an oligonucleotide, such as an EON as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide.

A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the EON as disclosed herein are shown in Intl. Patent Application Publication Nos. WO2020/154342, WO2020/154343, and WO2020/154344, which are herein incorporated by reference in their entireties.

In one embodiment, the EON herein may comprise one or more nucleotides carrying a 2′-MOE ribose modification. Also, in one embodiment, the EON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the EON comprises 2′-OMe ribose modifications at the positions that do not comprise a 2′-MOE ribose modification, and/or wherein the oligonucleotide comprises deoxynucleotides at positions that do not comprise a 2′-MOE ribose modification. In one embodiment the EON comprises one or more nucleotides comprising a 2′ position comprising a 2′-MOE, 2′-OMe, 2′-OH, 2′-deoxy, TNA, 2′-fluoro (2′-F), 2′,2′-difluoro (diF) modification, 2′-fluoro-2′-C-methyl modification, or a 2′-4′-linkage (i.e., a bridged nucleic acid such as a locked nucleic acid (LNA or examples mentioned in e.g. Intl. Patent Application Publication No. WO2018/007475)). In another embodiment, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in Intl. Patent Application Publication Nos. WO2016/097212, WO2017/220751, WO2018/041973, WO2018/134301, WO2019/219581, WO2019/158475, and WO2022/099159 for instance, which are herein incorporated by reference in their entireties. In all cases, the modifications should be compatible with editing such that the EON fulfils its role as an editing producing oligonucleotide that can form a double stranded complex with the target RNA and recruit a deaminating enzyme, that can subsequently deaminate the target adenosine. Where a monomer comprises an unlocked nucleic acid (UNA) ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a LNA).

A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy) ribosyl sugar.

The nucleobases in an EON herein can be adenine, cytosine, guanine, thymine, or uracil or any other moiety able to interact with another nucleobase through H-bonds, polarized bonds (such as CF) or aromatic electronic interactions. The nucleobases at any position in the nucleic acid strand can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose).

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide analog is not adenosine, guanosine, cytidine, thymidine, uridine, deoxyadenosine, deoxyguanosine, deoxycytidine, deoxythymidine, or deoxyuridine. In one embodiment, the uridine analog that may be the orphan nucleotide in the EON is iso-uridine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because phosphodiester bonds connect neighboring monomers together, they are often referred to as ‘backbone linkages’. It is understood that when a phosphate group is modified so that it is instead an analogous moiety such as a PS, such a moiety is still referred to as the backbone linkage of the monomer. This is referred to as a ‘backbone linkage modification’. In general terms, the backbone of an oligonucleotide comprises alternating scaffolds and backbone linkages.

EONs herein can comprise linkage modifications. A linkage modification can be, but not limited to, a modified version of the phosphodiester present in RNA, such as PS, chirally pure PS, (R)-PS, (S)-PS, methyl phosphonate (MP), chirally pure methyl phosphonate, (R)-methyl phosphonate, (S)-methyl phosphonate, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts and free acid forms are also included, as well as 3′→3′ and 2′→5′ linkages.

In one embodiment, an EON comprises a substitution of one of the non-bridging oxygens in the phosphodiester linkage. This modification slightly destabilizes base-pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Many of these non-naturally occurring modifications of the linkage, such as PS are chiral, which means that there are Rp and Sp configurations, known to the person skilled in the art. In one embodiment, the chirality of the PS linkages is controlled, which means that each of the linkages is either in the Rp or in the Sp configuration, whichever is preferred. The choice of an Rp or Sp configuration at a specified linkage position may depend on the target sequence and the efficiency of binding and induction of providing RNA editing. However, if such is not specifically desired, a composition may comprise oligonucleotides as active compounds with both Rp and Sp configurations at a certain specified linkage position. Mixtures of such EONs are also feasible, wherein certain positions have preferably either one of the configurations, while for other positions such does not matter.

Again, in all cases, the modifications should be compatible with editing such that the EON fulfils its role as an editing producing oligonucleotide that can, when attached to its target sequence recruit an adenosine deaminase enzyme because of the dsRNA nature that arises. In all aspects of the invention, the enzyme with adenosine deaminase activity is preferably ADAR1, ADAR2, or ADAT. In a highly preferred embodiment, the EON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. The disclosure also relates to a pharmaceutical composition comprising the EON as characterized herein, and a pharmaceutically acceptable carrier.

Other chemical modifications of the EONs herein include the substitution of one or more than one of any of the hydrogen atoms with deuterium or tritium, examples of which can be found in e.g., Intl. Patent Application Publication No. WO2014/022566 or WO2015/011694, which are herein incorporated by reference in their entireties.

The disclosure further relates to an EON herein, or a pharmaceutical composition comprising an EON herein, for use in the treatment or prevention of a disorder of the CNS related to lowered GABAergic inhibition, preferably caused by reduced functionality of KCC2. In one embodiment, the disclosure relates to an EON herein, or a pharmaceutical composition comprising an EON herein, for use in the treatment or prevention of a CNS disease related to lowered GABAergic inhibition, preferably caused by reduced KCC2 functionality. In one embodiment, the disclosure relates to an EON herein, or a pharmaceutical composition comprising an EON herein, for use in the treatment or prevention of pain or epilepsy, preferably caused by reduced KCC2 functionality.

EONs herein preferably do not include a 5′-terminal 06-benzylguanosine or a 5′-terminal amino modification and preferably are not covalently linked to a SNAP-tag domain (an engineered 06-alkylguanosine-DNA-alkyl transferase). Likewise, EONs herein preferably do not comprise a boxB RNA hairpin sequence. In one embodiment, an EON herein comprises 0, 1, 2 or 3 wobble base pairs with the target sequence, and/or 0, 1, 2, 3, 4, 5, 6, 7, or 8 mismatching base pairs with the target RNA sequence. No mismatch exists when the orphan nucleotide is uridine. One alternative for uridine is positioning an iso-uridine opposite the target adenosine, which likely does not pair like G pairs with U. Preferably, the target adenosine in the target sequence forms a mismatch base pair with the nucleoside in the EON that is directly opposite the target adenosine.

It should be noted that when an EON is delivered through a vector, for instance an AAV vector, chemical modifications are not present in the EON that acts on the target RNA molecule. Although it is preferred to use ‘naked’ EONs that have chemical modifications as outlined herein, EONs that are delivered through other means, for instance through AAV vector expression, or editing molecules that are circular, or have hairpin structures (recruiting portions, e.g., as disclosed in Intl. Patent Application Publication Nos. WO2016/097212, WO2017/050306, WO2020/001793, WO2017/010556, WO2020/246560, and WO2022/078995, which are herein incorporated by reference in their entireties) are also encompassed by the disclosure because these can also be applied to edit adenosines in the target SLC12A5 RNA molecule to generate a KCC2 protein with increased GABAergic inhibition activity.

An EON herein can utilise endogenous cellular pathways and naturally available ADAR enzymes to specifically edit a target adenosine in the target RNA sequence. An EON herein is capable of recruiting ADAR and complex with it and then facilitates the deamination of a (single) specific target adenosine nucleotide in a target RNA sequence. Ideally, only one adenosine is deaminated. An EON herein, when complexed to ADAR, preferably brings about the deamination of a single target adenosine.

Analysis of natural targets of ADAR enzymes has indicated that these generally include mismatches between the two strands that form the RNA helix edited by ADAR1 or 2. It has been suggested that these mismatches enhance the specificity of the editing reaction (Stefl et al. 2006. Structure 14(2):345-355; Tian et al. 2011. Nucleic Acids Res 39(13):5669-5681). Characterization of optimal patterns of paired/mismatched nucleotides between the EONs and the target RNA also appears important to the development of efficient ADAR-based EON therapy.

As outlined above, an EON herein makes use of specific nucleotide modifications at predefined spots to ensure stability as well as proper ADAR binding and activity. These changes may vary and may include modifications in the backbone of the EON, in the sugar moiety of the nucleotides as well as in the nucleobases or the phosphodiester linkages, as outlined in detail herein. They may also be variably distributed throughout the sequence of the EON. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the EON, while in other parts they should be avoided so as not to disrupt crucial interactions of the enzyme with the phosphate and 2′-OH groups. Specific nucleotide modifications may also be necessary to enhance the editing activity on substrate RNAs where the target sequence is not optimal for ADAR editing. Previous work has established that certain sequence contexts are more amenable to editing. For example, a target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42(10):e87), with a 5′ guanosine (G) being the least favored surrounding. The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle), is disfavored because the guanosine base sterically clashes with an amino acid side chain of ADAR2. In one preferred embodiment, targeting a 5′-CAC-3′ target sequence (as is the case for human SLC12A5 transcripts, see FIG. 1A), wherein the middle A is the target adenosine for deamination, is performed with an EON that comprises a Central Triplet (as it is sometimes referred to, even though it is not necessarily at a ‘central’ position in the oligonucleotide) opposite these three nucleotides that comprises a deoxyinosine (dl) on 3′ side of the orphan nucleotide. The orphan nucleotide, in one preferred embodiment, is dZ, which is a nucleotide carrying a Benner's base, with 2′-H at the ribose sugar (=DNA). In another embodiment, the orphan nucleotide is a cytidine comprising a 2′-F substitution or a 2′,2′-difluoro substitution in the ribose sugar. In another embodiment, the orphan nucleotide is a deoxyuridine or a nucleotide carrying a modified uracil nucleobase (such as iso-uridine) and wherein the 2′ position of the ribose sugar is 2′-H (=DNA). In a preferred embodiment the three nucleotides opposite a 5′-CAC-3′ triplet in a target molecule form a 5′-moeG-dZ-dl-3′ triplet in the EON, wherein ‘moeG’ is a guanosine comprising a 2′-MOE substitution in the ribose sugar moiety.

The disclosure relates to RNA editing oligonucleotides, generally referred to as “EONs” herein, that can bring about deamination of an adenosine in the SLC12A5 transcript, with a resulting KCC2 protein that has an increased functionality, preferably because of a diminished phosphorylation state. This means that the disclosure is not strictly limited to deamination of an adenosine in wildtype SLC12A5, but that other (single or multiple) adenosines may be targeted, which may also result in increased KCC2 protein function. Other adenosines may be identified, for instance by genetic screening in the population, or in silico, that are also important (or may become more important) for KCC2 function, and that also may be targeted through RNA editing, following the teaching of the present disclosure. All such RNA events and oligonucleotides that can be used for such targeting are encompassed by the disclosure, no matter what the exact nucleic molecule, or EON, looks like.

Mutagenesis studies of human ADAR2 revealed that a single mutation at residue 488 from glutamate to glutamine (E488Q), gave an increase in the rate constant of deamination by 60-fold when compared to the wild-type enzyme (Kuttan & Bass. Proc Natl Acad Sci USA 2012. 109(48):3295-3304). During the deamination reaction, ADAR flips the edited base out of its RNA duplex, and into the enzyme active site (Matthews et al. 2016). When ADAR2 edits adenosines in the preferred context (an A:C mismatch) the nucleotide opposite the target adenosine is the ‘orphan cytidine’. The crystal structure of ADAR2 E488Q bound to double stranded RNA (dsRNA) revealed that the glutamine (Gln) side chain at position 488 can donate an H-bond to the N3 position of the orphan cytidine, which leads to the increased catalytic rate of ADAR2 E488Q. In the wild-type enzyme, wherein a glutamate (Glu) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. Intl. Patent Application Publication No. WO2020/252376, which is herein incorporated by reference in its entirety, discloses the use of EONs with modified RNA bases, especially at the position of the orphan cytidine to mimic the hydrogen-bonding pattern observed by the E488Q ADAR2 mutant. By replacing the nucleotide opposite the target adenosine in the EON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. J Org Chem 2009. 74(21):8021-8030; Burchenal et al. (1976) Cancer Res 36:1520-1523) and Benner's base Z (also referred to as ‘dZ’; Yang et al. Nucl Acid Res 2006. 34(21):6095-6101) that were initially selected because they offer hydrogen-bond donation at N3 with minimal perturbation to the shape of the nucleobase. Benner's base is also chemically referred to as a 6-amino-5-nitro-3-yl-2(1H)-pyridone nucleobase. The presence of the cytidine analog in the EON may exist in addition to modifications to the ribose 2′ group. The ribose 2′ groups in the EON can be independently selected from 2′-H (i.e., DNA), 2′-OH (i.e., RNA), 2′-OMe, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker.

In one embodiment, a nucleotide analogue or equivalent within the EON comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

An EON herein is normally longer than 10 nucleotides, preferably more than 11, 12, 13, 14, 15, 16, still more preferably more than 17 nucleotides. In one aspect the EON herein is longer than 20 nucleotides. The EON herein is preferably shorter than 100 nucleotides, still more preferably shorter than 60 nucleotides, still more preferably shorter than 50 nucleotides. In a preferred aspect, the EON herein comprises 18 to 70 nucleotides, more preferably comprises 18 to 60 nucleotides, and even more preferably comprises 18 to 50 nucleotides. Hence, in a particularly preferred aspect, the EON herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. In one embodiment, the EON is 27, 28, 29, or 30 nucleotides in length.

In one aspect, at either end or both termini of an EON herein inverted deoxyT or dideoxyT nucleotides are incorporated.

As described above, in some embodiments the disclosure provides an EON for forming a double-stranded complex with a human SLC12A5 target RNA molecule in a human neuron cell, for instance in the brain. Thus, the therapeutic effect is preferably on a human neuronal cell in vivo. Of course, the methods may also be carried out in vitro or ex vivo.

The disclosure provides an EON herein, or pharmaceutical composition herein, for use in the treatment of disease. The disclosure also provides the use of an EON herein, or pharmaceutical composition herein, in the manufacture of a medicament for the treatment of disease. The disclosure also provides a method for treating a disease in a patient, comprising administering a therapeutically effective amount of an EON herein or a pharmaceutical composition herein. Preferably the disease is a disease caused by lowered GABAergic inhibition, due to an increased concentration of chloride in the neuronal cell, generally caused by a lowered activity of the key chloride extruder KCC2. The EON is administered therapeutically or prophylactically because both types of treatment could be beneficial.

After RNA editing has occurred in a cell, the modified RNA can become diluted over time, for example due to cell division, limited half-life of the edited RNAs, etc. Thus, in practical therapeutic terms a method herein may involve repeated delivery of an EON herein until enough target RNAs have been modified to provide a tangible benefit to the patient and/or to maintain the benefits over time.

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

### EXAMPLES

### Example 1. Editing of a Target Adenosine in a Human SLC12A5 Target RNA Molecule Using an In Vitro Biochemical Editing Assay

First, an initial set of 78 SLC12A5-targeting EONs was designed (shown in FIG. 1A) from which several were tested to address editing of human SLC12A5 target (pre-) mRNA in an in vitro biochemical editing assay (BEA). To obtain the SLC12A5 target RNA a PCR was performed using an SLC12A5 G-block (IDT) which contains the sequence for the T7 promotor and (a part of) the sequence of SLC12A5 as template using forward primer 5′-CTC GAC GCA AGC CAT AAC AC-3′ (SEQ ID NO: 106) and reverse primer 5′-TGG ACC GAC TGG AAA CGT AG-3′ (SEQ ID NO: 107). The 5′ to 3′ G-block sequence (SEQ ID NO: 108) was as follows, in which the target adenosine is underlined and in bold, and in which the primer positions are underlined:

TCTGGCTCGACGCAAGCCATAACACTAATACGACTCACTATAGGGAGGAG
CCTGAGGGGGAAGGGGAGACAGATCCGGAGAAGGTGCATCTCACCTGGAC
CAAGGACAAGTCGGTGGCAGAGAAGAATAAGGGCCCCAGTCCTGTCTCCT
CTGAGGGCATCAAGGACTTCTTCAGCATGAAGCCGGAGTGGGAGAACTTG
AACCAGTCCAACTACGTTTCCAGTCGGTCCACGTTTG

The PCR product was then used as template for the in vitro transcription. The MEGAscript T7 transcription kit was used for this reaction. The RNA was purified on a urea gel and then extracted in 50 mM Tris-Cl pH 7.4, 10 mM EDTA, 0.1% SDS, 0.3 M NaCl buffer and subsequently phenol-chloroform purified. The purified RNA was used as target in the BEA.

Initially, EONs B1 to B20 (see FIG. 1A) were annealed to the SLC12A5 target RNA, which was done in a buffer (5 mM Tris-Cl pH 7.4, 0.5 mM EDTA and 10 mM NaCl) at the ratio 1:3 of target RNA to oligonucleotide (600 nM oligonucleotide and 200 nM target). In the content of the present disclosure, references to the disclosed EON as B-1 to B-78 (with a dash) are equivalent and interchangeable with B1 to B78 (without a dash). The samples were heated at 95° C. for 3 min and then slowly cooled down to RT. Next, the editing reaction was carried out. The annealed oligonucleotide/target RNA was mixed with protease inhibitor (cOmplete™, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH 7.4, 1.5 mM EDTA, 3% glycerol, 60 mM KCl, 0.003% NP-40, 0.5 mM DTT, 40 mM K-glutamate and 3 mM MgSO₄) such that their final concentration was 6 nM oligonucleotide and 2 nM target RNA. The reaction was started by adding purified ADAR2 (GenScript) to a final concentration of 9 nM into the mix and incubated for predetermined time points at 37° C. Each reaction was stopped by adding 95 μl of 95° C. 3 mM EDTA solution. A 6 μl aliquot of the stopped reaction mixture was then used as template for cDNA synthesis using Maxima reverse transcriptase kit (Thermo Fisher) with random hexamer primer (ThermoFisher Scientific). Initial denaturation of RNA was performed in the presence of the primer and dNTPs at 95° C. for 5 min, followed by slow cooling to 10° C., after which first strand synthesis was carried out according to the manufacturer's instructions in a total volume of 20 μl, using an extension temperature of 62° C. Products were amplified for pyrosequencing analysis by PCR, using the Amplitaq gold 360 DNA Polymerase kit (Applied Biosystems) according to the manufacturer's instructions, with 1 μl of the cDNA as template using forward primer 5′-AGGAGCCTGAGGGGGAAG-3′ (SEQ ID NO: 109) and a biotinylated reverse primer 5′-GGGGCCCTTATTCTTCTCTGC-Biotin-3′ (SEQ ID NO:110).

Then PCR was performed using the following thermal cycling protocol: Initial denaturation at 95° C. for 5 min, followed by 45 cycles of 95° C. for 30 sec, 62° C. for 30 sec and 72° C. for 30 sec, and a final extension of 72° C. for 7 min.

Because inosines base-pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and data analysis was performed by the PyroMark Q48 Autoprep instrument (QIAGEN) following the manufacturer's instructions with 10 μl input of the PCR product and 4 μM sequencing primer: 5′-GGGAAGGGGAGACAG-3′ (SEQ ID NO: 111).

The analysis performed by the instrument provided the results for the selected nucleotide as a percentage of adenosine and guanosine detected in that position, and the extent of A-to-I editing at a chosen position was therefore measured by the percentage of guanosine in that position.

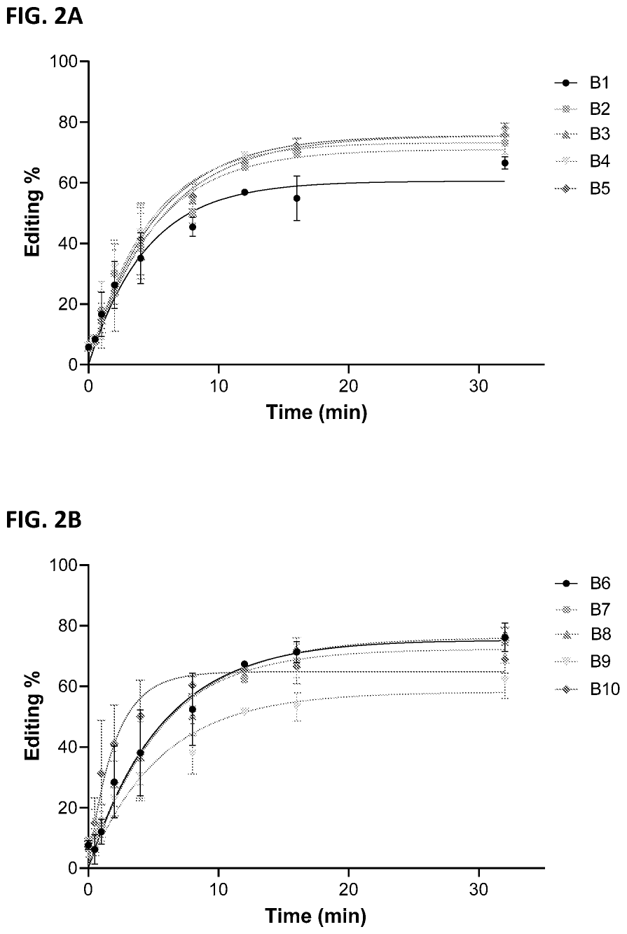

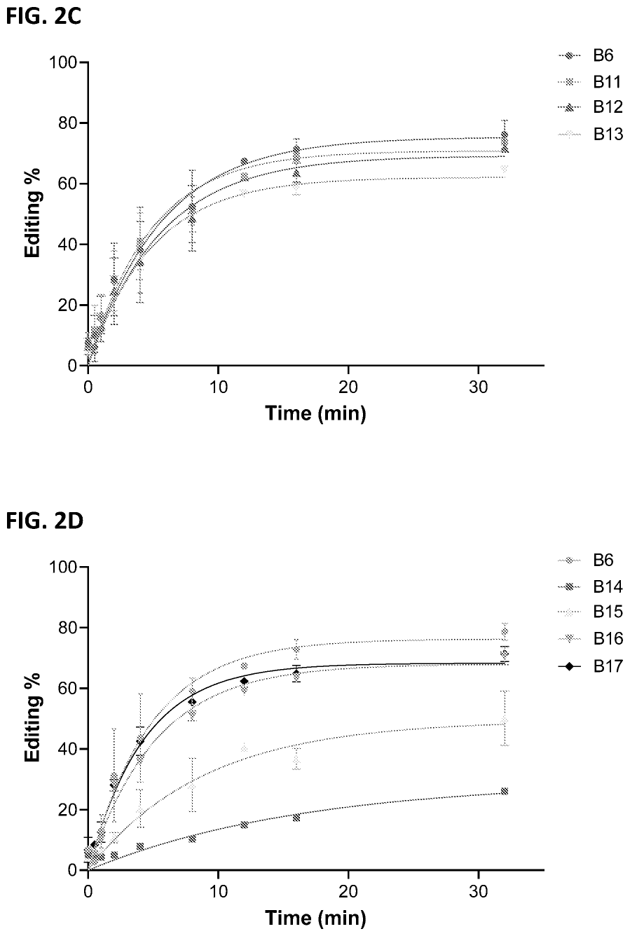

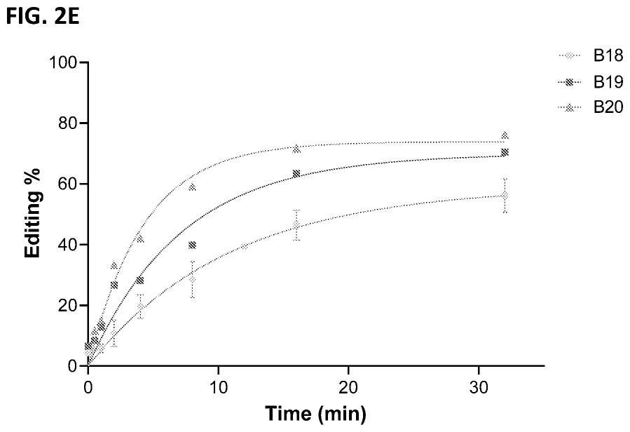

Results are provided in FIGS. 2A-2E, in which the results with EON B6 (29 nt) are given in FIGS. 2B, 2C, and 2D because it gave the best and fastest editing percentages. The editing percentages observed using these initial twenty EONs in the BEA clearly show that deamination of the target adenosine representing the adenosine in the ACC codon encoding the threonine at position 1007 in the human KCC2b isoform is feasible.

### Example 2. Editing of a Target Adenosine in a Human SLC12A5 Target RNA Molecule Using Retinal Organoids

To explore the possibility of editing human SLC12A5 transcript molecules in a more in vivo-resembling environment, the expression of KCC2 was determined in human retinal organoids that were generated as described in Intl. Patent Application Publication NO. WO2022/090256. Transcripts for KCC2 were detectable (approximately 1200 per cell) and KCC2 protein could be seen using a variety of anti-KCC2 antibodies in immunohistochemistry and using western blotting (data not shown), indicating that the retinal organoids, even though these do not represent brain tissue, do contain neuronal cells and are useful to determine whether SLC12A5 transcripts could be edited in a cellular environment.

In an initial screen, EONs B1 to B13 and B15 to B20 were tested for RNA editing. For this, 10 μM EON was added to the medium of an approximate 200-day old human wild type retinal organoid and incubated for 14 days. Two days after EON addition, the organoids were washed with fresh medium (EON in the medium were herewith removed) and medium was subsequently replaced every 2 days thereafter. RNA was extracted from the organoids and editing percentages were then determined using ddPCR.

RNA was isolated from the samples using the Direct-zol RNA Microprep Kits (Zymo) and eluted in 15 μL RNAse free water. Then, 500 ng of the RNA was used as a template for cDNA synthesis using the Maxima first strand cDNA synthesis kit (Thermo scientific) with random hexamers and OligoDt according to the manufacturer's instructions. The total reaction volume was 20 μl.

As inosines pair with cytidines during the cDNA synthesis in the reverse transcription reaction, the nucleotides incorporated in the edited positions during PCR will be guanosines. The percentage of guanosine (edited) versus adenosine (unedited) was defined by ddPCR in exon 23 of the SLC12A5 transcript.

Each ddPCR sample contained 1×ddPCR supermix for probes (no dUTP) (from Biorad), 0.9 μM forward primer 5′-GTGCAGCTGATCCACGAT-3′ (SEQ ID NO:112); 0.9 UM reverse primer 5′-GCCCTTATTCTTCTCTGCCA-3′ (SEQ ID NO:113); 0.6 UM of each double quenched WT unedited transcript probe 5′-HEX-TGCATCT+C+A+CCTGGA-3′ (SEQ ID NO:114); and mutant edited transcript probe 5′-Fam-TGCATCT+C+G+CCTGG-3′ (SEQ ID NO: 115) with the +sign denoting a locked nucleic acid (LNA) at the 3′ side; and template cDNA in a total volume of 21 μL. Droplets were made from the PCR mixes using the QX200 droplet generator (Biorad). Next, the droplet PCR was performed in a T100 thermal cycler (Biorad) with a heated lid of 105° C. and a ramp temperature of 2° C. per second. The polymerase was heat activated at 95° C. for 10 minutes. Each cycle the denaturation was performed at 95° C. for 30 seconds and the annealing/extension was performed at 59° C. for 60 seconds. This was repeated for 40 cycles in total. The enzymes were deactivated at 98° C. for 10 minutes and the reaction was held at 8° C. Fluorescent signal from the droplets was measured by the QX200 droplet reader (Biorad). Determining the number of fluorescent positive droplets and subsequent absolute quantification was performed with QuantaSoft software (Bio-Rad). As an additional quality control samples were removed which significantly deviated from the rest in terms of absolute copy numbers. This was determined by calculating the average number of total SLC12A5 (A+G transcripts) copies for each condition, samples were removed if they had a five-fold lower or higher number of SLC12A5 copies compared to the average.

Each sample was measured in duplicate with the average values used to determine the editing percentage. The editing percentage was calculated by dividing the number of edited (G) SLC12A5 transcript copies per well with the total number of SLC12A5 copies per well (A+G transcripts).

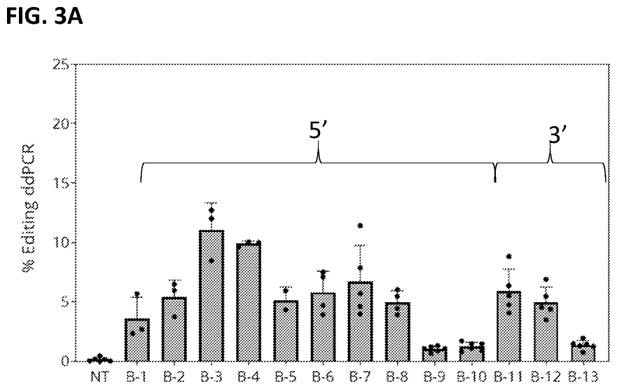

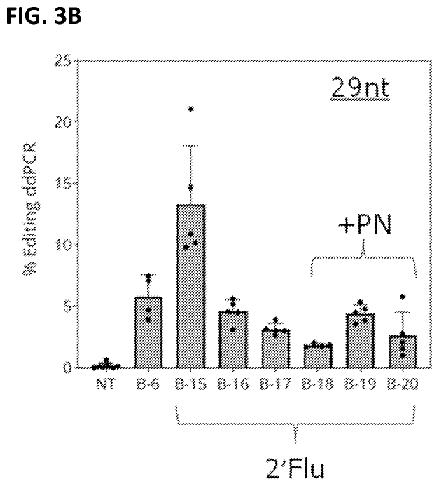

The results are shown in FIGS. 3A and 3B and clearly indicate that with certain EONs (e.g., B3, B4, and B15) editing of the SLC12A5 target transcript reached almost 15%.

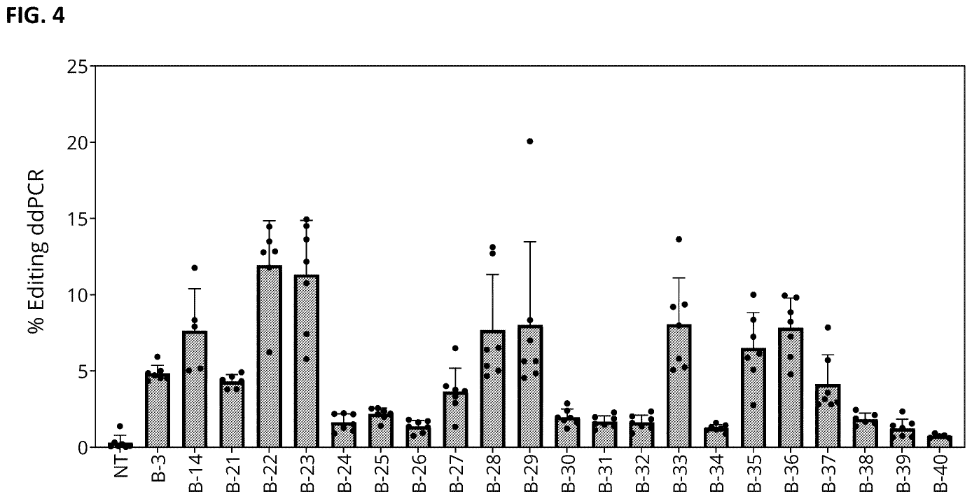

In a next screen, 20 other EONs (B21 to B40, see FIG. 1A) were used for RNA editing in human retinal organoids, again without using any transfection, and compared to EON B3 that was used in the first screen and EON B14. Setup and ddPCR procedures were as described above. The results of the second screen are given in FIG. 4 and indicate that editing percentages, when compared to B3, could be further increased, with EONs B22 and B23 providing the highest RNA editing percentages. These results show that the inventors were able to deaminate the target adenosine indicated in FIG. 1A in the SLC12A5 target transcript to a significant level, making it feasible that the KCC2 protein encoded by the transcript will lack the phosphorylation site at position 1007 (in the KCC2b isoform) and that thereby KCC2 activity can be increased.

### Example 3. Editing of a Human SLC12A5 Transcript in KCC2-Overexpressing HEK Cells

It was initially studied in what available cell lines or cellular systems enough expression of KCC2 could be detected to assess editing of the adenosine in codon ACC that codes for threonine at position 1007 of the KCC2b isoform. It appeared that from more than 10 available cell lines none expressed KCC2 to useful levels. However, retinal organoids expressed KCC2 levels such that they could be used for an initial in vitro screen (see Example 2). But since organoids are not conveniently cultured and generated and require complicated culturing conditions and growth factors, a new cell line was generated based on Human Embryonic Kidney 293 (HEK293) cells that received a stable expression construct, thereby stably over-expressing human SLC12A5 transcripts (and KCC2 protein). The expression construct was based on pcDNA3.1Neo with an open reading frame present downstream of a CMV promoter, using general methods that are well-known to the person skilled in the art. The resulting cell line named “HEK-KCC2” over-expressed KCC2 to very significant levels (expression data not shown) and could be used in subsequent RNA editing experiments.

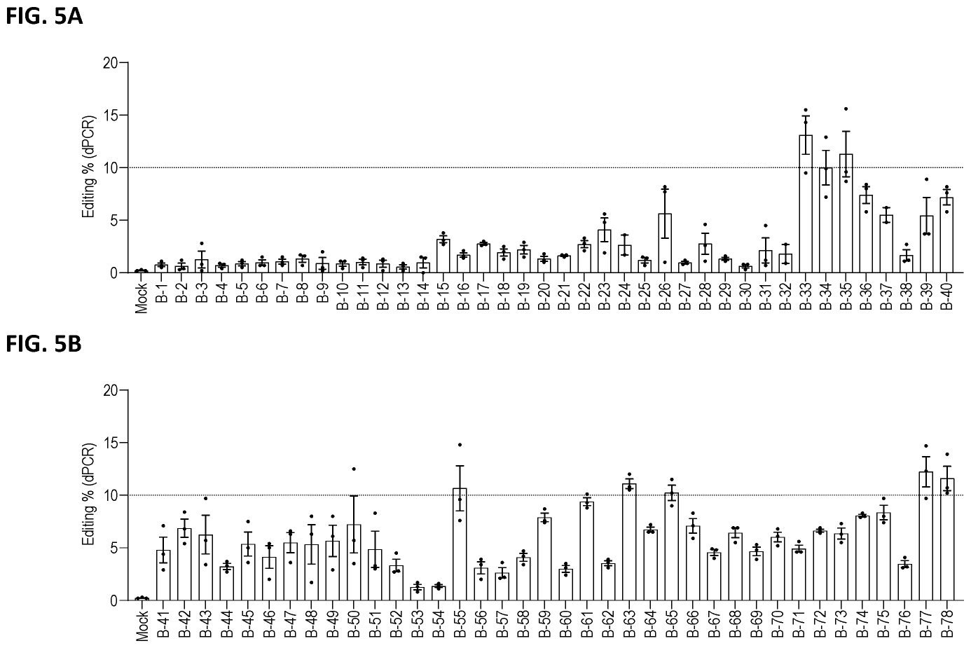

For an initial in vitro editing experiment, all 78 EONs shown in FIG. 1A were transfected into the HEK-KCC2 cells. For this, 79000 cells per cm² were plated and maintained in DMEM with 10% FBS and 750 μg/ml geneticin. Immediately after plating cells were transfected (also known as reverse transfection) using Lipofectamine®3000 (Thermo Fisher) with 200 nM EON using an EON and lipofection reagent ratio of 1:3 according to the manufacturers protocol. The editing was determined 24 hrs after the start of transfection, RNA was isolated using the Direct-zol RNA isolation kit according to the manufacturer's instructions (Zymo Research), and cDNA generation and dPCR analysis was performed as described for the retinal organoids above. The results are shown in FIGS. 5A and 5B. Certain EONs, especially B33, B34, B35, B50, B55, B63, B65, B77, and B78, performed relatively good, with editing levels reaching 15%. From these results in the context of the modifications that were introduced into the EONs (see FIG. 1A) no clear conclusions could be drawn in respect of positions of modifications and specific types of modifications and positions, except that it appears beneficial to have one or more 2′-F modified nucleotides in the EON. All the good-performing EONs have multiple 2′-F modified nucleotides in their sequence, some with consecutive stretches. Also, PNdmi linkages do not specifically inhibit or induce RNA editing based on the EON length and sequence content that was selected.

### Example 4. Detection of Phosphorylated Human KCC2 Protein Versus Non-Phosphorylated Human KCC2 Protein Using Antibodies

It was questioned whether the effect of editing, which should result in the occurrence of a KCC2 mutant that cannot be phosphorylated at position 1007 (in the KCC2b isoform) could also be determined by addressing the phosphorylated state of the protein. In principle, the amount of phosphorylated KCC2 should go down upon RNA as disclosed herein. It should be noted that the KCC2 protein, even when it is wild type and comprises a threonine residue at position 1007 can exist in a phosphorylated as well as in a non-phosphorylated state. Prior to assessment of the phosphorylation state of a naturally expressed KCC2 protein at position 1007 the entire protein needs to be immunoprecipitated away from other proteins because the peptide surrounding the target residue is conserved between a variety of proteins. However, such is not required in the HEK-KCC2 cell line. A competition experiment using 22-aa long peptides either comprising a phosphorylated Thr at position 12, a non-phosphorylated Thr at position 12, or a non-phosphorylated Ala (the mutant representing the edited protein) at position 12 was used to determine which could disturb the binding of the antibody to the phosphorylated KCC2 protein. It turned out that the Thr1007 phospho-KCC2 antibody (1:1000 dilution, Thermo Fisher, PA5-95677, polyclonal) did indeed specifically recognize the phosphorylated KCC2 protein and not the non-phosphorylated KCC2 protein (data not shown), now enabling one to distinguish between editing and non-editing events.

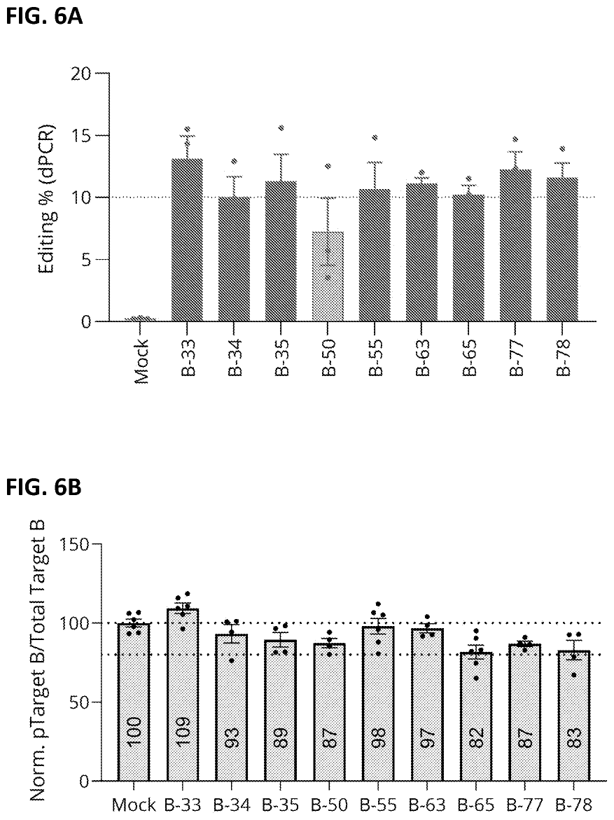

To test whether RNA editing could lower the amount of phosphorylated KCC2 in cell, the HEK-KCC2 cells were transfected with EONs B33, B34, B35, B50, B55, B63, B65, B77, and B78 using the same transfection methods as described above. Cells were harvested 48 hrs after start of transfection and editing percentages were determined generally as described above. Furthermore, the anti-KCC2 antibodies were used to determine the levels of non-phosphorylated and phosphorylated protein, wherein the background expression of β-tubulin was taken as a control. The editing results are shown in FIG. 6A. The editing observed for all 9 EONs appeared somewhat comparable again reaching 15% in the best cases. The results of the determination of the amount of phosphorylated KCC2 in these same cell samples is provided in FIG. 6B, indicating that the transfection of the indicated EONs, in comparison to the standard set by the mock transfection (set as 100%) decreased the amount of phosphorylated KCC2 in almost all cases, with the decrease caused by EONs B65 and B78 in fact being a significant drop. This suggests that RNA editing of the target adenosine in the SCL12A5 transcript causing a gain-of-function mutation by introducing an alanine at position 1007 instead of a threonine can lower the amount of phosphorylated KCC2, thereby providing a potential means to activate KCC2 to higher levels.

### Example 5. Editing of SLC12A5 Transcripts in Human Neurons Induced from Pluripotent Stem Cells (iPSC Neurons)

As noted above, KCC2 expression was very low in most cell lines, except for retinal organoids. This finding was the main driver to generate a HEK cell line that over-expressed human KCC2. However, it was noted that expression of the transcript fluctuated significantly over time and in different cultures. It was then found that neurons cultivated from induced pluripotent human stem cells (herein further referred to as ‘iPSC neurons’) expressed useful levels of human KCC2, enabling one to address RNA editing and potentially downstream effects.

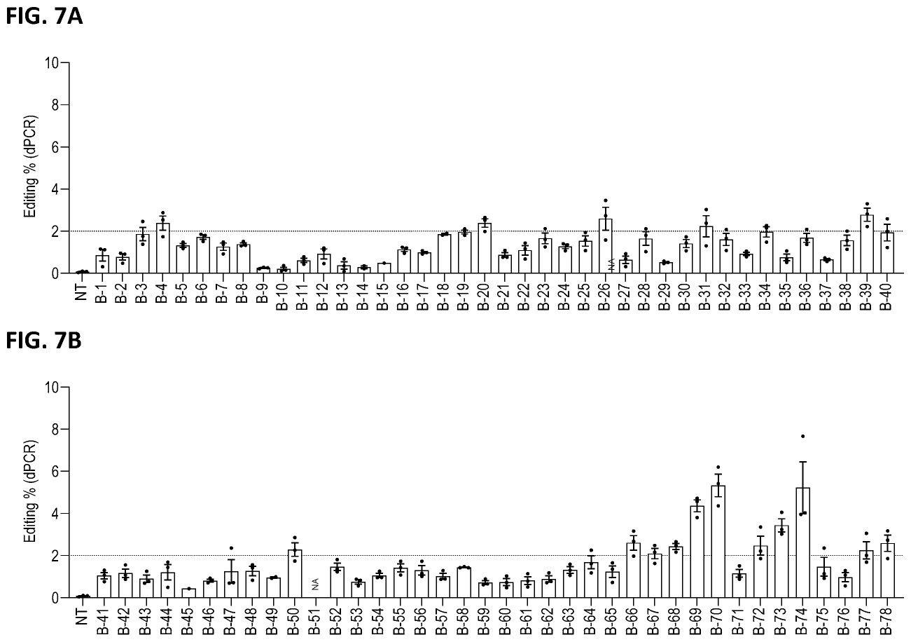

In a first experiment, the 78 EONs displayed in FIG. 1A were used in a gymnotic exposure experiment (gymnotic uptake=without transfection means; only adding EON to the culture medium). Herein, 5 μM EON was administered to the culture medium and kept on the cells for 48 hrs, after which the EON was slowly washed out through a replacement of 50% of the culture medium with fresh medium every 2 days. Editing was determined at 14 days after start of the experiment. RNA was isolated using miRVana RNA isolation kit (Thermo Fisher), the manufacturer protocol was followed with a few adaptations to remove the EONs from RNA samples. After disruption of the cells, the supplied miRNA homogenate additive was added at 1/10 of the total volume. After phenol:chloroform (same volume) extraction the upper aqueous phase was heated for 1 min at 60° C. 100% ethanol was added to the aqueous phase at ⅓ of the total volume. The samples were added to the supplied filter columns, after centrifugation, the flow through was discarded. The filters containing the mRNA were washed according to the protocol and eluted using 30 μl 95° C. nuclease-free water. The purified RNA was subsequently treated with TURBO DNase (Thermo Fisher) according to the supplied protocol. cDNA generation and dPCR analysis was performed as described above. Results are shown in FIGS. 7A and 7B. Interestingly, using this gymnotic uptake procedure rather than an active transfection assay, other EONs appeared to work more efficient than found in the HEK-KCC2 cells. Especially EONs B26, B39, B66, B68, B69, B70, B72, B73, B74, and B78 performed particularly well, with B78 being an EON that also performed good in the HEK-KCC2 cells.

### Example 6. Editing of SLC12A5 Transcripts in iPSC Neurons Using EONs with a Variety of 2′-Ribose Modifications

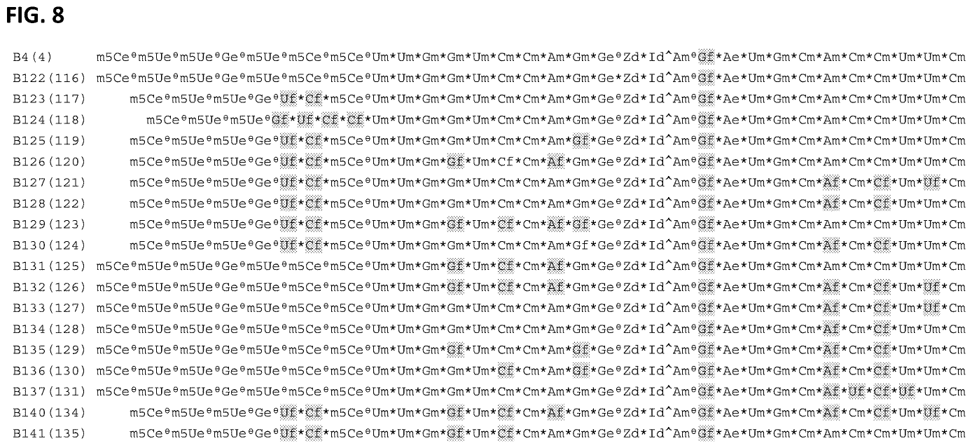

Then, to further investigate the potential role of modifications at the 2′ position of the ribose in the nucleotides of the SLC12A5 targeting EONs, and to see whether changing these could improve the level of editing in iPSC neurons, a new set of EONs was designed in which one or more positions in comparison to B4 (see FIG. 1A) comprised a 2′-F substitution. These EONs and their specific modifications are shown in FIG. 8. B138 (SEQ ID NO: 132) and B139 (SEQ ID NO: 133) could initially not being produced and were not tested in the first experiment. The SEQ ID NO's of these EONs are provided within brackets. B122 is identical to B4, except for the PS linkage at linkage position 0 (where B4 comprises a PO linkage). The 2′-F modified nucleotides are given with grey boxes, showing that B122 only has a single 2′-F modified nucleotide (at position-3), which was also present in all EONs B123 to B137 and B140 to B141.

5 μM EON was administered to iPSC neurons for gymnotic uptake as described above. The same washout treatment was applied by replacing 50% of the medium every two days. Culturing was continued for 14 days and RNA isolation using the mirVana RNA isolation kit, cDNA generation and dPCR analysis for editing was performed as described above.

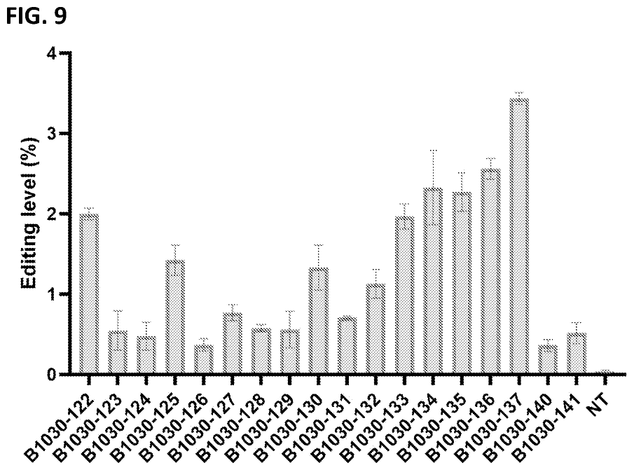

The results are shown in FIG. 9, indicating that several EONs, especially those that contain 3, 4 or 5 2′-F modified nucleotides at the 3′ side of the orphan nucleotide, with 2 or less (such as 0) 2′-F modified nucleotides at the 5′ side of the orphan nucleotide performed particularly well. Examples of these best performers include B134, B135, B136, and B137. In contrast, it appears that having multiple 2′-F modified nucleotides on either side of the orphan nucleotide, such as with B140, does not contribute to high editing percentages. It appears that having a series of 2′-F modified nucleotides on the 3′ side from the orphan nucleotide, while having low numbers of 2′-F modified nucleotides on the 5′ side of the orphan nucleotide, is beneficial to obtain higher editing percentages in respect of the SLC12A5 target transcript.

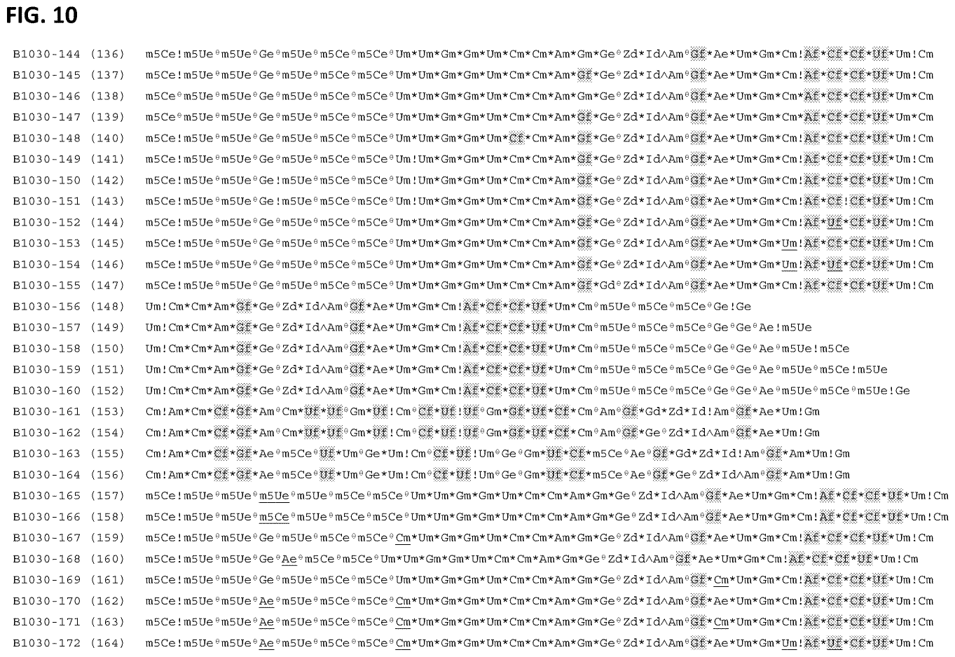

Based on these results, in which B137 performed best, B137 was used a base design to see whether further 2′-F modifications could increase editing percentages even further. Besides 2′-F and certain PNdmi linkage positions, also certain mismatches with the human target sequence and a-symmetrical designs were introduced. For this, a new set of EONs was designed (B144 to 172) shown in FIG. 10. The Zd position is the orphan nucleotide, in all cases. All 2′-F modified nucleotides are given again in grey boxes. Nucleotides that are not complementary to the human target sequence are underlined. B155, B161, and B163 comprise a central triplet (orphan nucleotide and its directly 3′ and 5′ adjacent nucleotides) that is 3×DNA. B156 to B160 have an a-symmetrical design in which the 5′ part seen from the orphan nucleotide is shorter than the 3′ part seen from the orphan nucleotide. B161 to B164 have a reverse a-symmetrical design. B161 to B164 also have a high number of 2′-F modified nucleotides in the 5′ part, seen from the orphan nucleotide. B165 to B172 have numerous nucleotides that do not match with the human target sequence.

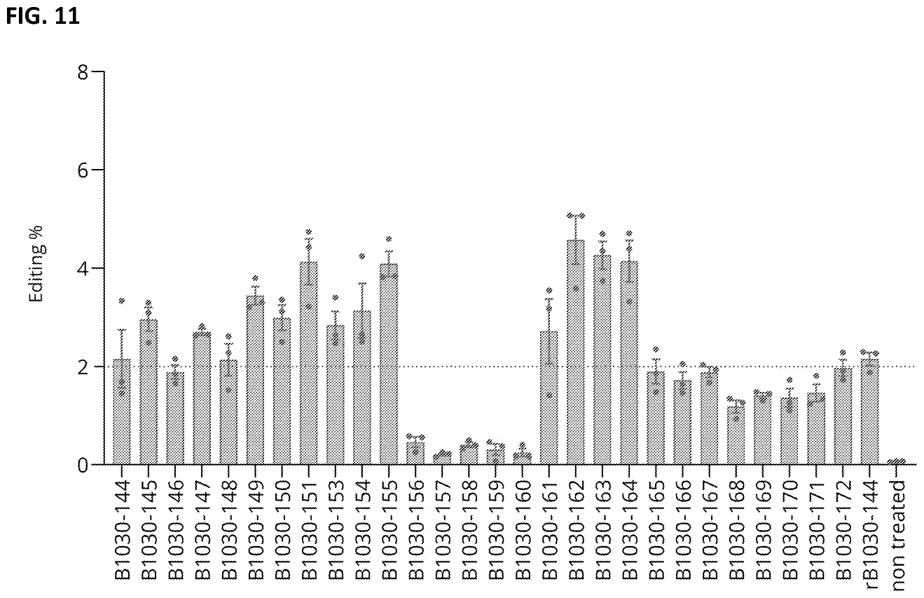

B144 to B172 (except for B152 that was initially not manufactured for unknown reasons) were tested in human iPSC neurons as discussed above, using 5 UM of the EONs and in a washout setup for 2 weeks. A rat Slc12a5 sequence-specific version of B144 was taken along (referred to in the figures as rB1030-144). The editing percentages obtained in this initial experiment are shown in FIG. 11, from which it becomes immediately clear that the a-symmetrical design in which there are a lower number of nucleotides on the 5′ side of the orphan nucleotide than there are nucleotides on the 3′ side (B156 to B160) significantly underperform in comparison to B144, which is an EON that resembles B137 (see above; except that in three positions a PNdmi linkage is introduced). In general, the a-symmetrical design in which there are a higher number of nucleotides on the 5′ side of the orphan nucleotide, in comparison to the 3′ side of the orphan nucleotide (B161 to B164) provide higher percentages of editing than B144, indicating that this is a preferred design. In the best performing EON, referred to as B162, there are only 6 nucleotides 3′ from the orphan nucleotide. Notably, also B151 and B155 perform significantly good, and these have the orphan nucleotide somewhat positioned in the centre of the EON. The presence of 3×DNA in the central triplet in B155 does not seem to hamper proper editing. B151 has a 2′-F pattern that resembles the 2′-F pattern used in B137 and B144.

### Example 7. Editing of Slc12a5 Transcripts in the Spinal Cord of Rats

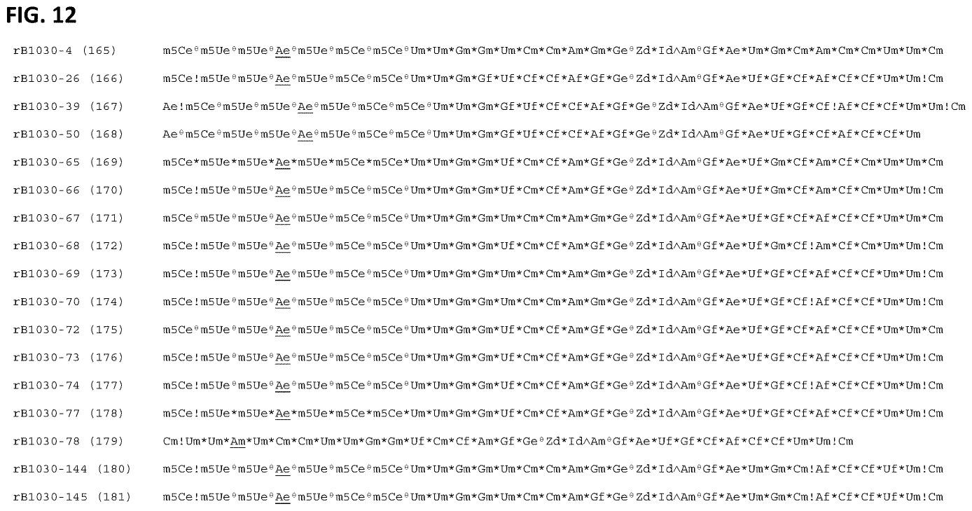

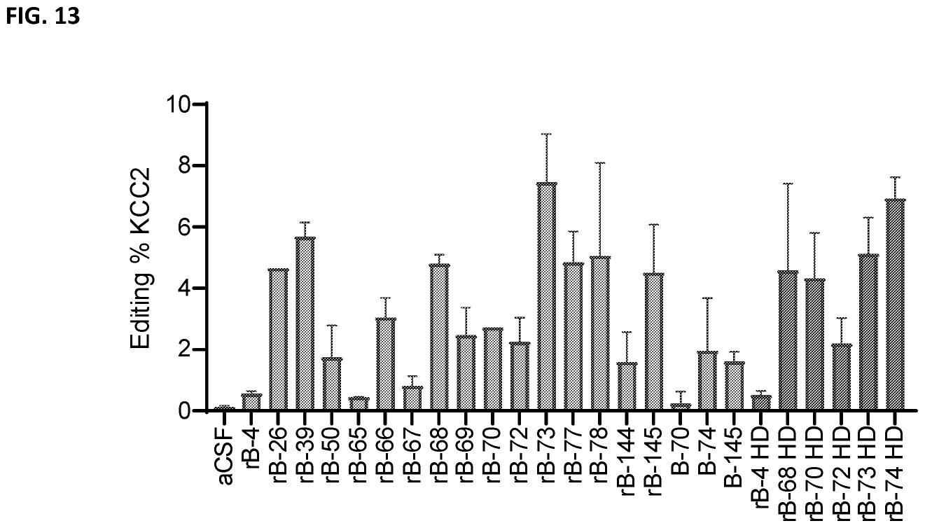

In a next in vivo experiment, it was investigated whether administering EONs to the spinal cord fluid would result in editing of the endogenous rat Slc12a5 transcript in the lumbar spinal cord and cortex. First, a set of rat-specific EONs with chemical modifications was designed, based on the results with the human SLC12A5 transcript specific EONs (see above). These rat-specific EONs are provided in FIG. 12. The numbering refers to their equivalent used in human iPSC neurons (see above), with the only difference between the human counterparts and the rat-specific EONs underlined, which is an A at position +14 in the EONs, being opposite a U in the rat target sequence. An EON targeting rat App transcripts was taken as a negative control, as well as spinal fluid from a non-treated animal. Sprague Dawley rats were taken as the study subject and 300 μg EON was injected intrathecally using a single dose. The following EONs were tested: rB-4 (rB1030-4; SEQ ID NO: 165), rB-26 (rB1030-26; SEQ ID NO:166), rB-39 (rB1030-39; SEQ ID NO: 167), rB-50 (rB1030-50; SEQ ID NO:168), rB-65 (rB1030-65; SEQ ID NO:169), rB-66 (rB1030-66; SEQ ID NO:170), rB-67 (rB1030-67; SEQ ID NO:171), rB-68 (rB1030-68; SEQ ID NO: 172), rB-69 (rB1030-69; SEQ ID NO:173), rB-70 (rB1030;70; SEQ ID NO:174), rB-72 (rB1030-72; SEQ ID NO:175), rB-73 (rB1030-73; SEQ ID NO:176), rB-74 (rB1030-74; SEQ ID NO: 177), rB-77 (rB1030-77; SEQ ID NO:178), rB-78 (rB1030-78; SEQ ID NO:179), rB-144 (rB1030-144; SEQ ID NO: 180), rB-145 (rB1030-145; SEQ ID NO:181), B-70 (SEQ ID NO:70), B-74 (SEQ ID NO:74), and B-145 (SEQ ID NO:137). Some EONs were tested with a higher dose: rB-4 (770 μg), rB-68 (575 μg), rB-70 (490 μg), rB-72 (605 μg), rB-73 (460 μg), and rB-74 (450 ug). Two weeks later, rats were sacrificed, and spinal fluid was withdrawn to determine editing of the equivalent A in the rat Slc12a5 transcript. Moreover, the rat brain was dissected, and the cortex was isolated. RNA was isolated using the mirVana RNA isolation kit (Thermo Fisher) as described above. cDNA synthesis and dPCR was performed generally as described above using the following rat-specific forward primer 5′-GTGCAGCTGATCCATGAC-3′ (SEQ ID NO: 182) and reverse primer 5′-GCCTTTGTTCTTCTGAGCCG-3′ (SEQ ID NO:183), the same detection probes were used as for human KCC2. It should be noted that administering a therapeutic to the spinal cord is a laborious and delicate procedure. In humans and monkeys, it is difficult to find and target the spinal cord in a single injection. With mice it is almost impossible to find the spinal cord because of its size, which is the reason why rats were selected for this study. It appeared that multiple injections failed (based on in situ imaging of the injection site after sacrifice; data not shown), and which becomes visible in the editing results that appeared close to 0 for the mis-injected dosages. Nevertheless, it turned out that in multiple cases administration was successful and editing could be detected. These results are provided in FIG. 13, showing that editing levels as high as 8% could be achieved in the spinal cord of rats, with rB-73 performing best. A dose higher than 300 μg (given in the figure as ‘HD’) did not give significant higher editing levels. Also here, some rats were mis-injected resulting in 0 editing levels. For this reason, samples in which no EON was detected in situ (in spinal cord tissue extracted from near the injection site) were not taken along for the calculations.

1. An RNA editing oligonucleotide (EON) capable of forming a double-stranded complex with a region of an endogenous human SLC12A5 transcript molecule in a cell, wherein the region of the SLC12A5 transcript molecule comprises a target adenosine, wherein the nucleotide in the EON that is directly opposite the target adenosine is the orphan nucleotide, wherein the counting of the nucleotides in the EON is such that the orphan nucleotide is number 0 and the nucleotides 5′ from the orphan nucleotide are positively (+) incremented towards the 5′ end and negatively (−) incremented towards the 3′ end, and wherein the double-stranded complex can recruit an endogenous ADAR enzyme to deaminate the target adenosine into an inosine, thereby editing the SLC12A5 transcript molecule.

2. The EON according to claim 1, wherein the SLC12A5 transcript molecule is a pre-mRNA or an mRNA molecule.

3. The EON according to claim 1 or 2, wherein the SLC12A5 transcript molecule has a wildtype sequence.

4. The EON according to any one of claims 1 to 3, wherein the target adenosine is in a codon encoding an amino acid that can be phosphorylated.

5. The EON according to claim 4, wherein the target adenosine is a first nucleotide of the codon encoding threonine at position 1007 of the SLC12A5-encoded KCC2b isoform.

6. The EON according to claim 4, wherein the target adenosine is a first nucleotide of the codon encoding threonine at position 1030 of the SLC12A5-encoded KCC2a isoform.

7. The EON according to any one of claims 1 to 6, wherein the deamination of the target adenosine results in an SLC12A5-encoded KCC2 protein with an increased activity.

8. The EON according to claim 7, wherein the increased activity results in a higher GABAergic inhibition.

9. The EON according to any one of claims 1 to 8, wherein the cell is a neuron, preferably a brain cell.

10. The EON according to any one of claims 1 to 9, wherein the EON is selected from the group consisting of SEQ ID NO: 1 to 104, and 116 to 164, preferably selected from the group consisting of SEQ ID NO:3, 4, 14, 15, 22, 23, 28, 29, 33, 34, 35, 36, 40, 55, 63, 65, 69, 70, 73, 74, 77, 78, 79, 80, 81, 82, 83, 84, 85, 86, 87, 88, 89, 90, 91, 92, 93, 94, 95, 96, 97, 98, 99, 100, 101, 102, 103, 104, 116, 119, 124, 127, 128, 129, 130, 131, 136, 137, 141, 142, 143, 145, 146, 147, 153, 154, 155, 156, and 184.

11. The EON according to any one of claims 1 to 10, comprising at least one non-naturally occurring chemical modification, and/or comprising one or more additional non-naturally occurring chemical modifications in the ribose, linkage, or base moiety, with the proviso that the orphan nucleotide is not a cytidine comprising a 2′-OMe ribose substitution.

12. The EON according to claim 11, wherein the orphan nucleotide is a deoxynucleotide comprising a 6-amino-5-nitro-3-yl-2(1H)-pyridone nucleobase, and the nucleotide on the −1 position in the EON is a deoxyinosine.

13. The EON according to claim 12, wherein the one or more additional modifications in the linkage moiety is each independently selected from a phosphorothioate (PS), phosphonoacetate, phosphorodithioate, methylphosphonate (MP), sulfonylphosphoramidate, mesyl phosphoramidate (PNms), or a (1,3-dimethylimidazolidin-2-ylidene) phosphoramidate (PNdmi) internucleotide linkage.

14. The EON according to claim 12 or 13, wherein the one or more additional modifications in the ribose moiety is a mono- or di-substitution at the 2′, 3′ and/or 5′ position of the ribose, each independently selected from the group consisting of:
—OH;
—F;
substituted or unsubstituted, linear or branched lower (C₁-C₁₀) alkyl, alkenyl, alkynyl, alkaryl, allyl, or aralkyl, that may be interrupted by one or more heteroatoms;
—O—, S—, or N-alkyl;
—O—, S—, or N-alkenyl;
—O—, S—, or N-alkynyl;
—O—, S—, or N-allyl;
—O-alkyl-O-alkyl;
-methoxy;
-aminopropoxy;
-methoxyethoxy;
-dimethylamino oxyethoxy; and
-dimethylaminoethoxyethoxy.

15. A vector, preferably a viral vector, more preferably an adeno-associated virus (AAV) vector, comprising a nucleic acid molecule encoding an EON comprising a sequence according to any one of SEQ ID NO: 79, 80, 81, 82, 83, 84, 85, 86, 87, 88, 89, 90, 91, 92, 93, 94, 95, 96, 97, 98, 99, 100, 101, 102, 103, 104, and 184, wherein the orphan nucleotide is a cytidine or a uridine, and the nucleotide at the −1 position in the EON is a guanosine.

16. A pharmaceutical composition comprising an EON according to any one of claims 1 to 14, or a vector according to claim 15, and a pharmaceutically acceptable carrier.

17. An EON according to any one of claims 1 to 14, or a vector according to claim 15, for use in the treatment of a disorder caused by a diminished GABAergic inhibition, preferably caused by a diminished KCC2 activity.

18. An EON for use according to claim 17, wherein the disorder is chronic pain or epilepsy.

19. Use of an EON according to any one of claims 1 to 14, or a vector according to claim 15, in the manufacture of a medicament for the treatment of a disorder caused by a diminished GABAergic inhibition, preferably caused by a diminished KCC2 activity.

20. Use according to claim 19, wherein the disorder is chronic pain or epilepsy.

21. A method of editing a SLC12A5 polynucleotide, the method comprising contacting the SLC12A5 polynucleotide with an EON capable of effecting an ADAR-mediated adenosine to inosine editing of a target adenosine in a codon encoding an amino acid that is associated with phosphorylation of the SLC12A5-encoded protein KCC2, thereby editing the SLC12A5 polynucleotide.

22. A method of treating a disorder caused by a diminished GABAergic inhibition, preferably caused by a diminished KCC2 activity, in an individual in need thereof, the method comprising contacting a SLC12A5 polynucleotide in a cell of the subject with an EON capable of effecting an ADAR-mediated adenosine to inosine editing of a target adenosine in a codon encoding an amino acid that is associated with phosphorylation of the SLC12A5-encoded protein KCC2, thereby treating the individual.

23. The method according to claim 21 or 22, wherein the target adenosine is a first nucleotide of the codon encoding threonine at position 1007 of the SLC12A5-encoded KCC2b isoform, or wherein the target adenosine is the first nucleotide of the codon encoding threonine at position 1030 of the SLC12A5-encoded KCC2a isoform.

24. A method of treating a disorder caused by a diminished GABAergic inhibition, preferably caused by a diminished KCC2 activity, the method comprising administering to an individual in need thereof a therapeutically effective amount of an EON according to any one of claims 1 to 14, a vector according to claim 15, or a pharmaceutical composition according to claim 16.

25. The method according to any one of claims 21 to 24, wherein the disorder is chronic pain or epilepsy.

26. A method of deaminating a target adenosine in an SLC12A5 pre-mRNA or mRNA molecule in a cell, the method comprising the steps of:
(i) providing the cell with an EON according to any one of claims 1 to 14;
(ii) allowing uptake by the cell of the EON;
(iii) allowing annealing of the EON to the SLC12A5 pre-mRNA or mRNA molecule;
(iv) allowing an endogenous ADAR enzyme to deaminate the target adenosine in the target RNA molecule to an inosine; and optionally
(v) identifying the presence of the inosine in the target RNA molecule.

27. The method according to claim 26, wherein the target adenosine is a first nucleotide of the codon encoding threonine at position 1007 of the SLC12A5-encoded KCC2b isoform, or wherein the target adenosine is the first nucleotide of the codon encoding threonine at position 1030 of the SLC12A5-encoded KCC2a isoform.

28. The method according to claim 26 or 27, wherein step (v) comprises:
a) determining the sequence of the SLC12A5 pre-mRNA or mRNA molecule;
b) assessing the presence of an SLC12A5-encoded KCC2 protein with a lower phosphorylation rate, preferably assessing the presence of KCC2 protein with an absent phosphorylation at position 1007 in the KCC2b isoform or at position 1030 in the KCC2a isoform; or
c) using a functional read-out, preferably assessing the level of GABAergic inhibition in the cell.

29. A nucleic acid molecule for editing a target adenosine in a human SLC12A5 pre-mRNA or mRNA molecule, wherein the target region is SEQ ID NO: 105, and wherein the target adenosine is the first nucleotide of the codon encoding threonine at position 1007 of the SLC12A5-encoded KCC2b isoform.

30. The nucleic acid molecule of claim 29, wherein the nucleic acid molecule is selected from the group consisting of SEQ ID NOS: 79, 80, 81, 82, 83, 84, 85, 86, 87, 88, 89, 90, 91, 92, 93, 94, 95, 96, 97, 98, 99, 100, 101, 102, 103, 104, and 184.

31. The nucleic acid molecule of claim 29 or 30, further comprising at least one non-naturally occurring chemical modification, and/or comprising one or more additional non-naturally occurring chemical modifications in a ribose, linkage or base moiety, with the proviso that the orphan nucleotide, which is the nucleotide in the nucleic acid that is directly opposite a target adenosine in the target region, is not a cytidine comprising a 2′-OMe ribose substitution.

32. The nucleic acid molecule of any one of claims 29 to 31, wherein the one or more additional modifications in the linkage moiety is each independently selected from a PS, phosphonoacetate, phosphorodithioate, MP, sulfonylphosphoramidate, PNms, or PNdmi internucleotide linkage.

33. The nucleic acid molecule of claim 31 or 32, wherein the one or more additional modifications in the ribose moiety is a mono- or di-substitution at the 2′, 3′ and/or 5′ position of the ribose, each independently selected from the group consisting of:
—OH;
—F;
substituted or unsubstituted, linear or branched lower (C₁-C₁₀) alkyl, alkenyl, alkynyl, alkaryl, allyl, or aralkyl, that may be interrupted by one or more heteroatoms;
—O—, S—, or N-alkyl;
—O—, S—, or N-alkenyl;
—O—, S—, or N-alkynyl;
—O—, S—, or N-allyl;
—O-alkyl-O-alkyl;
-methoxy;
-aminopropoxy;
-methoxyethoxy;
-dimethylamino oxyethoxy; and
-dimethylaminoethoxyethoxy.

34. A vector comprising a nucleotide sequence encoding the nucleic acid molecule of claim 29 or 30, wherein the orphan nucleotide is a cytidine or a uridine, and the nucleotide at the −1 position in the EON is a guanosine.
